(19) United States
(12) Patent Application Publication
(10) Pub. No.: US 2003/0119024 A1
(43) Pub. Date: Jun. 26, 2003

(54) Genes and proteins associated with T cell activation

(76) Inventors: Peter S. Linsley, Seattle, WA (US); Mao Mao, Redmond, WA (US); Matthew Biery, Bothell, WA (US)

Correspondence Address: PENNIE AND EDMONDS, 1155 AVENUE OF THE AMERICAS, NEW YORK, NY, 100362711

(21) Appl. No.: 10/201,481

(22) Filed: Jul. 19, 2002

Related U.S. Application Data
(60) Provisional application No. 60/306,968, filed on Jul. 20, 2001.

(51) Int. Cl. C12Q 1/68; C07H 21/04; C07K 14/705; C12N 5/08; C12P 21/02

(52) U.S. Cl. 435/6; 435/69.1; 435/320.1; 435/372; 530/350; 536/23.5

(57) ABSTRACT

The present invention relates to proteins associated with T cell activation, termed TCAPs (T Cell Activation-associated Proteins), TCAP-encoding genes and nucleic acid derived therefrom, and methods for identifying TCAP-encoding genes. The method provides amino acid sequences of the TCAPs TA-GAP, TA-GPCR, TA-PP2C, TA-NFKBH, TA-KRP, TA-WDRP, and TA-LRRP, and nucleotide sequences of the genes encoding them, and nucleic acid derived therefrom, as well as amino acid and nucleic acid derivatives (e.g., fragments) thereof. The invention further relates to fragments (and derivatives thereof) of particular TCAPs that comprise one or more domains of a TCAP. Antibodies to TCAPs, and to TCAP derivatives, are additionally provided. Methods of production of the TCAPs, derivatives, e.g., by recombinant means, are also provided. Therapeutic and diagnostic methods and pharmaceutical compositions are provided. In specific examples, isolated TA-GAP, TA-GPCR, TA-PP2C, TA-NFKBH, TA-KRP, TA-WDRP, and TA-LRRP genes from human, and the sequences thereof, are provided.

This application claims benefit of U.S. Provisional Application No. 60/306,968, filed Jul. 20, 2001, which is hereby incorporated by reference in its entirety.

### 1. FIELD OF THE INVENTION

The present invention relates to novel T cell activation-associated proteins (TCAPs), in particular to a G Protein-coupled Receptor (TA-GPCR), two GTPase-Activating Proteins (TA-GAP), a serine/threonine class 2C phosphatase (TA-PP2C); an NF-κB-like transcription factor (TA-NFKBH); a keich repeat-containing protein (TA-KRP); a transducin-like protein with a WD motif-containing domain (TA-WDRP); and a leucine repeat-rich protein (TA-LRRP); and derivatives thereof, the genes encoding them, and derivatives thereof. Production of proteins, derivatives, and antibodies is also provided. The invention further relates to therapeutic compositions and methods of diagnosis and therapy.

### 2. BACKGROUND OF THE INVENTION

### 2.1. GENE EXPRESSION IN T CELL ACTIVATION

The study of gene expression changes has played a major role in development of the understanding of T lymphocyte activation. During an immune response, T cells interact with antigen presenting cells (APCs) in a complex process involving intercellular interactions between many T cell surface receptors and cognate ligands on the APCs. During these encounters, T cells undergo an elaborate transcriptional response, leading to cellular differentiation and acquisition of immunologic function (Crabtree, Science 243:355-61(1989)). T cell activation also plays a central role in development of immunologic mechanisms of disease (W. Paul, ed., Fundamental Immunology, Third Edition, Raven Press, New York, 1993). An understanding of the molecular basis of T cell activation is therefore essential to both our understanding of immune responses and of how to manipulate them therapeutically. Gene expression changes accompanying T cell activation and differentiation have been the subject of numerous studies (Choi, et al., Cell. Immunol. 168(1):78-84 (1996); Zipfel, et al., Mol. Cell. Biol. 9(3):1041-8 (1989); Zheng & Flavell, Cell 89(4):587-96 (1997); Liu, et al., Genomics 39(2):171-84 (1997); Renner et al., J. Immunol. 159(3):1276-83 (1997); Ishaq, et al., J. Biol. Chem. 14:273(33):21210-16 (1998); Teague, et al., Proc. Natl. Acad. Sci. U.S.A. 96(22):12691-96 (1999); Hedrick, et al., Nature 308:149-53 (1984); Yanagi, et al., Nature 308:145-9 (1984); Brunet, Immunol. Rev. 103:21-36 (1988)).

Comparing patterns of gene expression is a widely used means of identifying novel genes, investigating gene function and finding potential new therapeutic targets (Shiue et al., Drug Devel. Res. 41:142-159 (1997)). The study of gene expression changes has played a major role in development of our understanding of T lymphocyte activation. With the completion of the human genome sequencing effort, it is now a realistic goal to document all gene expression changes that occur during T cell activation (Marrack, et al., Curr. Opin. Immunol. 12(2):206-9 (2000)), but it is more difficult to assess the relevance of these changes for immunologic function. Historically, many techniques have been used to identify and clone differentially expressed genes (Liang et al., Science 257:967-71 (1992); Welsh et al., Nucleic Acids Res. 20(19):4965-70 (1992); Tedder et al., Proc. Natl. Acad. Sci. U.S.A. 85(1):208-12 (1988); Davis et al., Proc. Natl. Acad. Sci. U.S.A. 81(7):2194-8 (1984); Lisitsyn et al., Science 259:946-51 (1993); Velculescu et al., Science 270:484-7 (1995); Diatchenko et al., Proc. Natl. Acad. Sci. U.S.A. 93(12):6025-30 (1996); Jiang et al., Proc. Natl. Acad. Sci. U.S.A. 97(23):12684-9 (2000); Yang et al., Nucleic Acids Res. 27(6):1517-23(1999)). However, these are generally not well suited for discerning the functional significance of gene expression differences. In many cases, these differences are not unique to a particular cellular pathway and the specificity of these changes becomes apparent only after secondary characterization using labor intensive techniques (Shiue et al., Drug Devel. Res. 41:142-159 (1997)).

Recently, the technique of DNA microarray hybridization has been used to quantify the expression of many thousands of discrete sequences in a single assay known as expression profiling (Wang et al., Gene 229(1-2):101-8 (1999); Schena et al., Science 270:467-470 (1995); Lockhart, et al., Nat. Biotechnol. 14:1675-1680 (1996); Lockhart et al., U.S. Pat. No. 6,040,138). Many applications have been described for expression profiling, but perhaps most relevant to elucidating gene function is the development of tools used to group genes according to similarities in patterns of gene expression in expression profiling experiments. Coexpression of genes of known function with poorly characterized or novel genes has been suggested as a method to assign function to genes for which information is not available (Eisen et al., Proc. Natl. Acad. Sci. U.S.A. 95(25):14863-8 (1998)). Using a reference database or compendium of expression profiles from Saccharomyces cerevisiae, novel open reading frames (ORFs) were used to show that coordinated transcriptional regulations were enriched for a given phenotype (Hughes et al., Cell 102:109-126 (2000)). In human cells, coregulation of uncharacterized expressed sequence tag (EST) sequences with known genes was noted, but no evaluation of the identities and properties of these ESTs was made.

### 2.2. G-PROTEIN COUPLED RECEPTORS

G-protein coupled receptors (GPCRs) form an extensive family of transmembrane regulatory proteins that elicit intracellular signals in nearly every physiological system of chordates and invertebrate organisms. These receptors are biologically important and malfunction of these receptors results in diseases such as Alzheimer's, Parkinson's, diabetes, dwarfism, color blindness, retinitis pigmentosa and asthma. GPCRs are also important signaling molecules in subjects with depression, schizophrenia, sleeplessness, hypertension, anxiety, stress, renal failure and in several other cardiovascular, metabolic, neural, oncology and immune disorders (Horn and Vriend, J. Mol. Med. 76:464-468 (1998)). They have also been shown to play a role in HIV infection (Feng et al., Science 272:872-877 (1996)).

GPCRs are integral membrane proteins characterized by the presence of seven hydrophobic transmembrane domains which span the plasma membrane and form a bundle of antiparallel alpha helices. The transmembrane domains account for structural and functional features of the receptor. In most cases, the bundle of helices forms a binding pocket; however, when the binding site must accommodate more bulky molecules, the extracellular N-terminal segment or one or more of the three extracellular loops participate in binding and in subsequent induction of conformational change in intracellular portions of the receptor. The activated receptor, in turn, interacts with an intracellular G-protein complex, composed of a heterotrimer of α, β and γ subunits, the a subunit having a bound guanosine diphosphate (GDP). Upon interaction of the G protein with the ligand-bound receptor, the G protein substitutes GTP for the GDP, causing a simultaneous release of the α subunit from the β and γ subunits, and the release of all three subunits from the receptor. The now-activated α subunit in turn mediates further intracellular signaling activities, generally through interaction with guanine nucleotide binding (G) proteins and the production of second messengers such as cyclic AMP (cAMP), phospholipase C, inositol triphosphate or ion channel proteins (Baldwin, J. M. Curr. Opin. Cell Biol. 6:180-190 (1994)). The activity of the receptors are modulated by modification, such as phosphorylation, or by binding to a regulatory molecule, such as by the negative regulatory molecule arrestin, or by internalization wherein the receptor is degraded in a lysosome (see generally Hu, L. A., et al., J. Biol. Chem. 275:38659-38666 (2000)).

The amino-terminus of the GPCR is extracellular, of variable length and often glycosylated, while the carboxy-terminus is cytoplasmic. Extracellular loops of the GPCR alternate with intracellular loops and link the transmembrane domains. The most conserved domains of GPCRs are the transmembrane domains and the first two cytoplasmic loops. GPCRs range in size from under 400 to over 1000 amino acids (Coughlin, S. R., Curr. Opin. Cell Biol. 6:191-197 (1994)).

GPCRs can be divided into five broad structural classes, A-E, based on amino acid sequence similarity and sequence motifs. The largest class is class A, which can, in turn, be divided into subgroups according to receptor sequence similarity and ligand characteristics. The categorization of these relationships is illustrated by the following examples:

Class A (rhodopsin-like) GPCRs include: biogenic amine receptors (e.g., α-adrenergic, β-adrenergic, dopamine, histamine, muscarinic acetylcholine, melatonin, 5-HT, octopamine and tyramine); peptidic ligand receptors (e.g., angiotensin, bombesin, chemokine, endothelin, galanin, hormone protein, F-met-leu-phe, melanocortin, N-formyl peptide, neuropeptide Y, neurokinin, opiate, tachykinin, vasopressin, oxytocin and somatostatin); rhodopsin receptors (e.g., vertebrate rhodopsin, arthropod rhodopsin, and olfactory receptors); prostanoid receptors (e.g., prostaglandin, prostacyclin, and thromboxane); nucleotide receptors (e.g., adenosine and purinoceptors); hormone-releasing GPCRs (e.g., gonadotropin-releasing hormone, thyrotropin-releasing hormone, growth hormone, and secretagogue GPCRs);

Class B (secretin-like) GPCRs include calcitonin, calcitonin releasing factor, calcitonin gene-related peptide, gastrin, cholecystokinin, glucagon, growth hormone-releasing hormone, parathyroid hormone, vasoactive intestinal peptide, PACAP, diuretic hormone and secretin GPCRs;

Class C (metabotropic glutamate-like) GPCRs include metabotropic glutamate, metabotropic GABA_{B}, and extracellular calcium-sensing GPCRs;

Class D includes pheromone GPCRs; and

Class E includes cAMP-binding GPCRs.

GPCRs respond to a diverse array of ligands including lipid analogs, amino acids and their derivatives, peptides, cytokines, and specialized stimuli such as light, taste, and odor. GPCRs function in physiological processes including vision (the rhodopsins), smell (the olfactory receptors), neurotransmission (muscarinic acetylcholine, dopamine, and adrenergic receptors), and hormonal response (luteinizing hormone and thyroid-stimulating hormone receptors).

In addition, GPCR mutations, both of the loss-of-function and of the activating variety, have been associated with numerous human diseases (Coughlin, supra). For instance, retinitis pigmentosa may arise from either loss-of-function or activating mutations in the rhodopsin gene. Somatic activating mutations in the thyrotropin receptor cause hyperfunctioning thyroid adenomas (Parma, J. et al. Nature 365:649-651 (1993)). Parma et al. suggest that certain G-protein-coupled receptors susceptible to constitutive activation may behave as proto-oncogenes.

### 2.3. RHO-GTPASE ACTIVATING PROTEINS

GAPs (GTPase activating proteins) greatly increase the rate of GTP hydrolysis by Gα proteins and are thus responsible for terminating G protein activation by returning Fα to the GDP-bound state (Kehrl et al., Immunity 8:1-10 (1998); Berman et al., J. Biol. Chem. 273:1269-1272 (1998)). GDP dissociation inhibitors (GDIs) inhibit GDP dissociation and are responsible for keeping the G protein in an inactive state in resting cells (Takai et al., Int. Rev. Cytol. 133:187-230 (1991); Bokoch et al., FASEB J. 7:750-759 (1993)). GDP dissociation stimulators (GDSs) stimulate the exchange of GDP for GTP and thereby promote Gα activation (Takai et al., Int. Rev. Cytol. 133:187-230 (1991); Bokoch et al., FASEB J. 7:750-759 (1993)).

A superfamily of GTPases known as Ras proteins has been found to be critical in the regulation of normal and transformed cell growth, and control much of the information flow within the cell. Rho proteins are members of the Ras superfamily of GTPases, and are involved in the organization of the cytoskeleton. Rho activity is regulated by the opposing actions of GTPase-activating proteins (GAPs) and guanine nucleotide exchange factors (GEFs), with GAPs stimulating the slow intrinsic rate of GTP hydrolysis on Ras and GEFs stimulating the basal rate of exchange of GDP for GTP on Ras. Thus, GAPs act as negative regulators of Ras function (Boguski & McCormick, Nature 366:643-654 (1993)).

GAPs can be specific to distinct physiological processes, but can also affect several processes through GTPase pathway crosstalk. At least one mammalian Rho-GAP has been characterized that contains a region related to the C terminal domain of Ber, a RhoGEF. Whereas some GAPs are specific for one kind of Rho, one GAP, p190, is a “promiscuous” GAP for all Rho proteins. Adding to the crosstalk due to some cross-specificity of particular GAPs, certain GAPs may interact with each other to mediate physiological changes. For example, p120-GAP binds p190-GAP, linking Ras with Rho proteins to cause changes in the cytoskeleton (Boguski & McCormick, Nature 366:643-654 (1993)).

### 2.4. SERINE/THREONINE CLASS 2C PHOSPHATASES

The class 2C serine/threonine protein phosphatases (PP2Cs), as the name suggests, remove phosphate groups from the serine and/or threonine residues of a wide variety of proteins. The dephosphorylation of phosphothreonine appears to be approximately 20-fold more efficient than dephosphorylation of phosphoserines, and it has been speculated that PP2C substrates are phosphorylated at threonine residues. The protein phosphatases have been separated into seven groups based on their biochemical properties (Herzig and Neumann, Physiol. Rev. 80(l):173-210 (2000)). PP2C is a monomeric protein of approximately 382 residues. Class 2C STPs exist in two isoforms, designated α and β; alternative splicing appears to generate the latter. Alternative splicing appears to further segregate the α and β isoforms into sub-isoforms (Deana et al., Biochim. Biophys. Acta 1051:199-202 (1990)).

PP2Cs have been implicated in a number of important biochemical pathways. In particular, it is implicated in the negative regulation of the MAP (mitogen activated protein) kinase signaling cascade. For example, PP2Cα2 is able to suppress the activation of p38 and JNK (Jun-N-terminal kinase) MAP kinases induced by environmental stress, wound stress and the cytokine TNF-α (Takekawa et al., EMBO J. 17:4744-4752 (1998)). Because serine/threonine phosphatases are involved in such important responses, they are attractive target of, and candidates for, small-molecule inhibition and pharmacological intervention (see e.g., Lazo et al. U.S. Pat. No. 6,040,323).

### 2.5. NF-κB-LIKE TRANSCRIPTION FACTORS

NF-κB proteins are transcription factors. In their inactive form, they are complexed with the IκBα protein in the cytoplasm. However, upon cell activation, they disassociate from IκBα, translocate to the nucleus and bind κB motifs in the promoters of many genes, in particular of the promoters of genes whose expression is involved the immune response. NF-κB has been implicated as a transcriptional activator in a variety of disease and inflammatory states and is thought to regulate cytokine levels including but not limited to TNF-α and also to be an activator of HIV transcription (Dbaibo, et al., J Biol. Chem. 17762-66 (1993); Duh et al., Proc. Natl. Acad. Sci. U.S.A. 86, 5974-78 (1989); Bachelerie et al., Nature 350:709-12 (1991); Suzuki et al., Biochem. Biophys. Res. Comm. 193:277-83 (1993)). In particular, the inappropriate regulation of NF-κB and its dependent genes has been associated with septic shock, graft-versus-host disease, acute inflammatory conditions, acute phase response, transplant rejection, autoimmune diseases, and cancer (Manna & Aggarwal, J. Immunol. 165:2095-2102 (1999)).

### 2.6. KELCH-LIKE PROTEINS

Members of the kelch-repeat superfamily of proteins all contain one or more copies of a domain known as a β propeller (see Adams et al., Trends Cell Biol. 10: 17-24 (2000)). The β propeller consists of 4-12 repeats of the kelch motif, each repeat constituting a “blade” of the propeller. Most members of the five categories of kelch repeats within the kelch superfamily have propellers having six kelch repeats (see Adams, supra, providing representative kelch motif sequences for each of the five categories). Kelch superfamily proteins engage in a wide variety of physiological functions, such as actin-binding, control of cell morphology and organization, and control of gene expression. Most keich proteins have protein binding partners, and in a number of proteins, it has been established that the β propeller facilitates the interaction (Adams, supra). Biochemical and mutational analyses provide evidence that the keich proteins as a class engage in multiprotein complexes through contact sites in their β propeller domains.

Kelch proteins regulating gene expression include the protein Keapl, which sequesters Nrf2 (NF-E2-related factor 2) transcription factor in the cytoplasm. Another kelch protein, RAG-2 (recombination activating gene 2) combines with RAG-1 to facilitate V(D)J recombination in immunoglobulin and T cell receptor genes. It is the N-terminal 355 amino acid residues of RAG-2, which form the β propeller, that interact with RAG-1 to cause recombination. Persons with mutations in the β propeller of RAG-2 suffer deficient RAG-1 DNA binding, and consequent severe combined immunity deficiency. The proteins currently characterized represent only a small part of a putatively extensive and growing superfamily (Adams, supra).

### 2.7. TRANSDUCINS AND WD DOMAINS

Transducin is a G protein essential for the exquisitely tightly-regulated transmission of visual information in the rods of the eye. Upon stimulation by a photon, rhodopsin, a prototypical GPCR, changes conformation to its active form, metarhodopsin, and is able to interact with transducin, a G protein consisting of three subunits, Gα Gβ and Gγ. Gα of transducin, like the α subunit of other G proteins, contains a bound GDP in its inactive state. Metarhodopsin binds transducin, causing the release of GDP and the binding of transducin to metarhodopsin. Subsequent Gα binding of GTP causes the release of Gα both from metarhodopsin and from Gβγ. Gα-GTP then activates its effector, cyclic GMP phosphodiesterase. The subsequent drop in the local concentration of cGMP causes closure of cGMP-gated channels in the photoreceptor plasma membrane (Natochin et al., J. Biol. Chem. 274(12):7865-7869 (1999); Marin et al., J. Biol. Chem. 275(3):1930-1936 (2000)).

Cessation of the photoresponse requires hydrolysis of the GTP on G_{α}-GTP.

However, the native GTPase activity of transducin is far too slow. The activity of transducin therefore, is tightly regulated by the protein RGS9-Gβ5L, which greatly increases the rate of GTP hydrolysis. As the transmission of visual signals is on a subsecond timescale, the activation of transducin by metarhodopsin, and the subsequent quenching of the signal by RGS9-Gβ5L occurs within a fraction of a second (Skiba et al., J. Biol. Chem. 275(42):32716-32720 (2000)).

While transducin is a functional part of the visual system, one transducin-like protein, transducin-like enhancer of split (TLE), has been shown to act as part of a transcriptional complex in liver-specific expression. TLE interacts with the CRII domain of the liver-specific pleiotropic transcription factor HNF3β to repress HNF3β-mediated transcription (Wang et al., J. Biol. Chem. 275(24):18418-18423 (2000)).

TLP also contains a domain defined by WD repeats. WD repeats are similar to the ketch repeats described above, in that the WD repeats together form the blades of a “propeller.” Conserved sequence motifs differentiate the WD repeat motif from that of the kelch motif. The best-characterized WD-repeat protein is the Gβ subunit of heterotrimeric g proteins, which forms a tight heterodimer with the γ subunit. The function of the WD repeat domain, in general, has been to facilitate the reversible interaction between the protein containing it and one or several other proteins (Smith et al., TIBS 24(5):181-5 (1999)).

Citation or discussion of references herein above shall not be construed as an admission that such references are prior art to the present invention.

### 2.8 LEUCINE-RICH REPEAT PROTEINS

Leucine-rich repeats (LRRs) are relatively short motifs (22-28 residues in length) found in a variety of cytoplasmic, membrane and extracellular proteins associated with widely different functions. LRRs appear to facilitate protein-protein interaction. In vitro studies of a synthetic LRR from Drosophila Toll protein have indicated that the peptides form gels by adopting beta-sheet structures that form extended filaments. These results are consistent with the idea that LRRs mediate protein-protein interactions and cellular adhesion (Gay et al., FEBS Lett. 291(l):87-91 (1991)). Other functions of LRR-containing proteins include binding to enzymes (Tan et al. J Biol Chem. 265(1):13-9 (1990) and vascular repair (Hickey et al., Proc. Natl. Acad. Sci. U.S.A. 86(17):6773-7 (1989). The 3-D structure of ribonuclease inhibitor, a protein containing 15 LRRs, reveals LRRs to be a new class of alpha/beta fold (Kobe et al., Nature 366(6457):751-6 (1993).

### 3. SUMMARY OF THE INVENTION

We have evaluated the use of coexpression over many reference conditions as a method for gene discovery and functional characterization of unknown expressed sequence tags (ESTs) coregulated over many conditions with T cell cytokines, which are well known markers for T cell activation. Transcripts associated with these ESTs have been identified that have been found to encode novel polypeptides with desirable properties for targets for immunosuppressive drugs, including a G protein-coupled receptor, two GTPase-activating proteins, a serine/threonine class 2C phosphatase, a keich motif-containing protein, two variants of an NF-κB-like transcription factor, a transducin-related protein with a WD motif-containing domain, and a leucine-rich repeat protein.

The present invention provides genes and proteins associated with T cell activation. Specifically, the invention relates to the T cell activation-associated proteins TA-GAP (a GTPase activating protein), TA-GPCR (a G protein-coupled receptor), TA-PP2C (a serine/threonine class 2C phosphatase), TA-NFKBH (an NF-κB like transcription factor), TA-KRP (a kelch repeat-containing protein), TA-WDRP (transducin-like protein), and TA-LRRP (a leucine repeat-rich protein), their amino acid sequences and the sequences of the genes and associated nucleic acids encoding them. These proteins are referred to herein as TCAPs (T Cell Activation-associated Proteins). Nucleic acids hybridizable to or complementary to the foregoing nucleotide sequences are also provided.

The invention also relates to a method of producing the proteins of the present invention, and of using these proteins as markers for T cell activation by antibody recognition. The invention also relates to probes for hybridization analysis, and primers for PCR analysis, of markers of T cell activation. TCAPs are upregulated during T cell activation; thus, the invention further relates to methods of regulating the immune response by modifying the activity of these proteins or the genes that encode them.

The invention also relates to nucleic acids containing full-length open reading frames encoding TCAPs, identified by the method of the invention.

The invention also relates to TCAP derivatives that are functionally active, i.e., they are capable of displaying one or more known functional activities associated with a full-length (wild-type) TCAP. Such functional activities include but are not limited to GTPase activation activity (TA-GAP), GTPase activity (TA-GPCR), G-coupled protein receptor activity (TA-GPCR), DNA binding activity (TA-NFKBH), protein binding activity (TA-WDRP, TA-NFKBH, TA-KRP, TA-LRRP), antigenicity (i.e., the ability to bind or compete with a TCAP for binding) to an anti-TCAP antibody, immunogenicity (ability to generate antibody which binds to a TCAP), and ability to bind, or to compete with TCAPs for binding, to a receptor/ligand for a particular TCAP. The invention further relates to derivatives (including but not limited to fragments) of TCAPs that comprise one or more domains of a TCAP.

Antibodies to TCAPs, or to their derivatives, are additionally provided. Because these antibodies detect specific proteins correlated with T cell activation, they detect specific markers of T cell activation.

The present invention further provides methods of production of the TCAPs and derivatives thereof, e.g., by recombinant means.

The present invention also relates to therapeutic and diagnostic methods and compositions based on TCAPs and associated nucleic acids. Therapeutic compounds of the invention include but are not limited to TCAPs and TCAP derivatives, including fragments thereof; antibodies thereto; nucleic acids encoding the TCAPs or derivatives thereof; and antisense nucleic acids to the genes encoding these two proteins. Diagnostic methods include but are not limited to the detection of diseases or disorders involving T cell activation or a lack thereof by measuring the expression of one or more TCAPs or TCAP nucleic acids, where increased expression of the TCAP(s) or TCAP nucleic acid(s), relative to a standard or control or subject not having the disorder, indicates the presence of a disease or disorder involving inappropriate or undesired T cell activation, and decreased expression, relative to a standard or control or subject not having the disorder indicates the presence of a disease or disorder involving a deficit in desired T cell activation. Diagnostic methods further include monitoring of the production, or suppression of production, of TCAPs by use of nucleic acids that hybridize to TCAP nucleic acids, and/or monitoring the production, or suppression of production, of TCAPs by use of antibodies that recognize at least one TCAP.

The invention provides for treatment or prevention of immune disorders involving inappropriate or undesirable T cell activation by administering compounds that antagonize TCAP activities (e.g., antibodies, antisense nucleic acids). The invention also provides methods of treatment or prevention of immune disorders involving failure of T cell activation, or by activation of T cells where such activation is desired, by administering compounds that promote TCAP activity, e.g., TA-GAP, TA-GPCR, TA-WDRP, TA-NFKBH, TA-PP2C, TA-KRP or TA-LRRP function (e.g., TA-GAP, TA-GPCR, TA-WDRP, TA-NFKBH, TA-PP2C, TA-KRP or TA-LRRP, an agonist of any of these TCAPs; nucleic acids that encode any of these TCAPs). In a specific embodiment, TCAP function is antagonized in order to suppress the activation of T cells, and thereby modify the immune response, in vivo or in vitro.

Animal models, diagnostic methods and screening methods for predisposition to disorders, and methods to identify TCAP agonists and antagonists, are also provided by the invention.

A novel T cell activation-associated protein from an activated Jurkat T cell line, TA-GAP has been identified. The cDNA sequence containing the full-length open reading frame encoding TA-GAP was identified through use of an EST (AI253155) that was co-regulated over many conditions with T cell cytokines. The nucleotide sequence of the cDNA containing the TA-GAP coding region has similarity to human BAC clone RP1-111C20 from chromosome 6q25.3-27, which clone contains part of a novel gene described as similar to that encoding Chlamydomonas radial spoke protein 3. The amino acid sequence of TA-GAP shows homology to the human KIAA1391 protein (GenBank Acc. No. BAA92629.1), whose function is not known, and to a human SH3 domain-binding protein that includes a RhoGAP (GTPase-activator protein for Rho-like GTPases). The invention thus provides the polynucleotide sequence of the cDNA for the two splice variants encoding TA-GAP (FIGS. 1, 2, SEQ ID NOS: 1, 2) and vectors and host cells comprising TA-GAP for use in immunosuppressive drug development. The invention also provides the amino acid sequence of two TA-GAP variants (FIGS. 1, 2, SEQ ID NOS: 3, 4), a method of recombinantly producing TA-GAP for use as a target, and a method for producing antibodies directed against TA-GAP.

Also identified is T Cell Activation-associated Protein TA-GPCR. TA-GPCR was identified by analysis of a transcript corresponding to the EST AA040696, which was co-regulated with cytokine transcripts. Through PCR of actual transcripts, two cDNAs containing full-length open reading frames were identified that encode the same protein, TA-GPCR. TA-GPCR shows homology to a putative chemokine receptor (GenBank Acc. No. NP_{—}006009.1) and a putative seven transmembrane spanning receptor of the rhodopsin family (GanBank Acc. No. CAC17790). The invention thus provides the nucleotide sequence of the two cDNAs encoding full-length TA-GPCR (FIGS. 3A-3D, 4A-4C; SEQ ID NOS: 5, 6) and vectors and host cells comprising a TA-GPCR-encoding nucleic acid sequence for use in immunosuppressive drug development. The invention also provides the amino acid sequence of TA-GPCR (FIGS. 3A-3D, 4A-4C; SEQ ID NO: 7).

Also identified in the same manner are: (1) TA-PP2C, predicted to be a serine/threonine class 2C phosphatase; (2) TA-NFKBH, an NF-κB like transcription factor containing five Ankyrin repeats; (3) TA-KRP, a protein containing a POZ/BTB domain and three kelch repeats; (4) TA-WDRP, a transducin-like protein containing 11 WD repeats; and (5) TA-LRRP, a protein containing four transmembrane-domains and 12 leucine-rich repeats. The invention thus provides the nucleotide sequence of cDNAs encoding the above full-length proteins (FIGS. 5-10; SEQ ID NOS: 8, 10, 12, 14, 16, 18, respectively) and vectors and host cells comprising a TA-PP2C-, TA-NFKIBH-, TA-KRP-, TA-WDRP-, or TA-LRRP-encoding nucleic acid sequence for use in immunosuppressive drug development. The invention also provides the amino acid sequence of TA-PP2C, TA-NFKBH, TA-KRP, TA-WDRP, and TA-LRRP (FIGS. 5-10; SEQ ID NO: 9, 11, 13, 15, 17, 19, respectively). These proteins, and the related genes, have not been previously identified.

### 3.1. DEFINITIONS

As used herein, underscoring or italicizing the name of a gene shall indicate the gene, in contrast to its encoded protein product which is indicated by the name of the gene in the absence of any underscoring or italicizing. For example, “TA-GPCR” shall mean the gene encoding the protein product “TA-GPCR.”

### 4. DESCRIPTION OF THE FIGURES

FIGS. 1A-1E show a 3218 nucleotide cDNA sequence (SEQ ID NO: 1) encoding TA-GAP and the predicted 731 amino acid-long sequence of TA-GAP (SEQ ID NO: 2).

FIGS. 2A-2D show a 3051 nucleotide cDNA sequence (SEQ ID NO: 3) encoding a splice variant of TA-GAP and the predicted 553 amino acid-long sequence of a variant of TA-GAP (SEQ ID NO: 4).

FIGS. 3A-3E show a 3612 nucleotide cDNA sequence (SEQ ID NO: 5) encoding TA-GPCR and the predicted 346 amino acid-long sequence of TA-GPCR (SEQ ID NO: 7).

FIGS. 4A-4D show a 2345 nucleotide cDNA sequence (SEQ ID NO: 6) encoding TA-GPCR and the predicted 346 amino acid-long sequence of TA-GPCR (SEQ ID NO: 7).

FIGS. 5A-5G show a 3748 nucleotide cDNA sequence (SEQ ID NO: 8) encoding TA-PP2C and the predicted 304 amino acid-long sequence of TA-PP2C (SEQ ID NO: 9).

FIGS. 6A-6C show an 1736 nucleotide cDNA sequence (SEQ ID NO: 10) encoding a long form of TA-NFKBH and the predicted 465 amino acid-long sequence of the long form of TA-NFKBH (SEQ ID NO: 11).

FIGS. 7A-7D show an 1834 nucleotide cDNA sequence (SEQ ID NO: 12) encoding a short form of TA-NFKBH and the predicted 313 amino acid-long sequence of the short form of TA-NFKBH (SEQ ID NO: 13).

FIGS. 8A-8F show a 3049 nucleotide cDNA sequence (SEQ ID NO: 14) encoding TA-WDRP and the predicted 951 amino acid-long TA-WDRP (SEQ ID NO: 15).

FIGS. 9A-9H show a 4617 nucleotide cDNA sequence (SEQ ID NO: 16) encoding TA-KRP and the predicted 575 amino acid-long TA-KRP (SEQ ID NO: 17).

FIGS. 10A-10E show a 3588 nucleotide cDNA sequence (SEQ ID NO: 18) encoding TA-LRRP and the predicted 803 amino acid-long TA-LRRP (SEQ ID NO: 19).

FIG. 11 diagrams the relative sizes of TA-GPCR, TA-GAP (long and short forms), TA-LRRP, TA-WDRP, TA-KRP, TA-NFKBH (long and short forms), and TA-PP2C. Specific domains or sequence motifs present in each are indicated as gray boxes.

FIG. 12 shows co-clustering of known cytokines and unknown ESTs in expression profiling experiments. FlexJet™ arrays representing either 25,000 or 50,000 Unigene clusters were hybridized to a mixture of cRNAs from untreated versus treated cells of various types. The experiments contained comparisons of activated and unactivated Jurkat cells; K562 cells; peripheral blood T cells; THP1 cells; NB4 cells; JCAM cells; HL60 cells; and B-lymphoblast cells. A total of 3853 genes regulated >3-fold, P<0.0l in a total of 104 experiments were analyzed by a two dimensional hierarchical clustering algorithm. Genes were grouped by greatest similarity of regulation over all experiments (Y axis) and the experiments showing the greatest similarities in gene regulation (X axis). Only a section of the total data set is shown (64 genes and 94 experiments). Experiments involving activated peripheral blood T cells and activated Jurkat T cells are indicated with horizontal black bars. Genes upregulated in a particular experiment are colored medium gray; genes down regulated in that experiment are colored light gray; and genes showing no regulation in a particular experiment are colored black. The set of genes shown here demonstrates enrichment for T cell cytokines. Known cytokine genes are highlighted on the right hand Y axis with light gray circles. This region also contains 21 ESTs of unknown function, indicated with dark gray circles.

FIG. 13 shows linkage of two Unigene clusters by genomic tiling.

FIG. 13A depicts the mapping of the consensus sequences from two previously unlinked Unigene clusters, Hs. 7581 and Hs. 130864 to a portion of human chromosome 6.

FIG. 13B depicts a portion of an array containing oligonucleotides from the genomic sequence surrounding two Unigene EST clusters, Hs. 7581 and Hs. 130864, on chromosome 6. Nested oligonucleotides (60 bp) were selected from every tenth nucleotide position of both strands of non-repetitive sequence in alternating fashion. The array was hybridized with a mixture of cRNA from activated (labeled with red fluorescent dye) and unactivated (labeled with green fluorescent dye) Jurkat cells. The red-fluorescing dots (shown as dark gray) represent oligonucleotides showing greater hybridization to a transcript expressed at higher levels in activated cells, whereas yellow spots (shown as white) show equal hybridization with both samples. The white circles show indicate the boundaries of a contiguous segment of genomic DNA hybridizing with a transcript present at higher levels in activated cells. The top circle maps near the 5′ end of Hs. 130864 and the bottom circle, near the 3′ end of Hs. 7581. The contiguous hybridization suggests that this region hybridizes with a single transcript.

FIG. 13C depicts a graph showing XDEV measurements of hybridization over the region of chromosome 6 adjacent to Unigene clusters, Hs. 7581 and Hs. 130864. For a description of the calculation of XDEV, see Example 3, infra. The region between the white circles from part B corresponds to the peak of XDEV measurements.

FIG. 13D depicts linking by tiling data of Unigene clusters, Hs. 7581 and Hs. 130864. The previously known boundaries of these clusters are shaded dark gray; the region between these (shown in white) was predicted to hybridize with the same transcript by hybridization data in FIGS. 6B and 6C. The linkage of these EST clusters was confirmed by RT-PCR analysis. Further extension of these EST clusters by RT-PCR analysis revealed that this genomic region represents an exon from the 3′ untranslated region of the human homolog of the transcription factor, Bach2.

FIG. 14 shows the upregulation of TA-GAP during T cell activation. Transcripts from activated Jurkat T cells showing significant regulation (>2-fold change and P<0.0001 in most samples) over the unactivated condition are depicted as thin light gray lines. R/G ratio is above 0.0 when a particular gene is upregulated. The TA-GAP transcript is depicted by the thick black line (indicated by the arrow); transcripts for 18 other GAP domain-containing proteins are depicted by thin black lines (KIAA1501, KIAA0660, A1479025, ABR, GIT1, GIT2, ARHGAP1, ARHGAP4, G38P, GAPCENA, GAPL, IQGAP1, IQGAP2, NGAP, RAB3GAP, RANGAP1, RAP1GA1, RASA1). Of the transcripts tested that encode GAP-domain containing proteins, TA-GAP is the only one to show significant upregulation during T cell activation.

FIG. 15 shows upregulation of TA-GPCR during T cell activation. Transcripts from activated Jurkat cells showing significant regulation (>2-fold change and P<0.0001 in most samples) are depicted as thin light gray lines. Transcripts encoding GPR proteins are depicted as black lines. The R/G ratio is above 0.0 when a particular gene is upregulated. The TA-GPCR transcript is depicted by the thick black line, and transcripts for 27 other GPR proteins are depicted by thin black lines (GPR39, GPR51, AI61367, AI208357, GPRK6, GPRK5, GPR51, GPR19, AI659657, GPR48, EBI2, GPRK5, GPRK6, GPR68, GPR4, GPR9, LANCL1, CCR1, CCR4, CCR5, CCR7, CCR8, CMKLR1, CXCR4, HM74, LTBR4, AA040696). Of the transcripts tested that encode GPRs, TA-GPCR was the only one to show significant upregulation.

### 5. DETAILED DESCRIPTION OF THE INVENTION

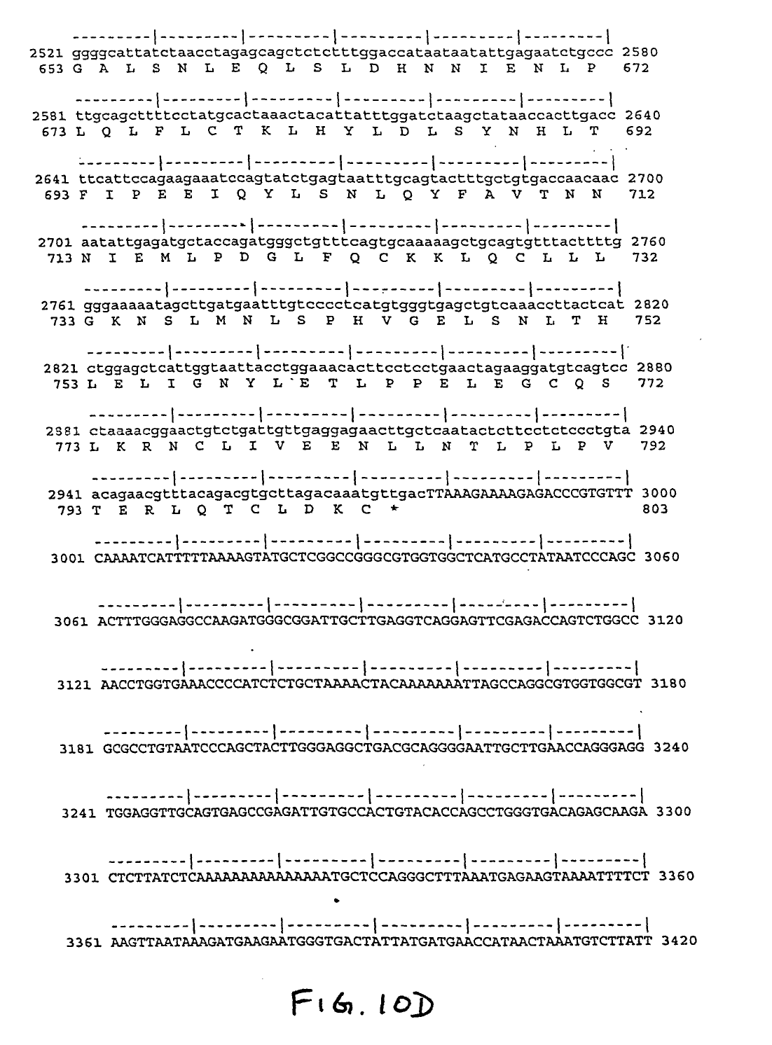

The present invention relates to the amino acid sequences of the T Cell Activation associated proteins TA-GAP, TA-GPCR, TA-PP2C, TA-NFKBH, TA-KRP, TA-WDRP and TA-LRRP (referred to hereinafter individually and as a group as “TCAPs”), and to nucleotide sequences of the genes encoding these proteins. SEQ ID NO: 1 is a cDNA sequence containing a full open reading frame that encodes TA-GAP (SEQ ID NO: 2). SEQ ID NO: 3 is a cDNA sequence containing a full open reading frame that encodes a splice variant of TA-GAP (SEQ ID NO: 4). TA-GAP has high sequence similarities to known GTPase-activating proteins, and likely possesses this function and is involved in the modulation of signal transduction. SEQ ID NO: 5 and 6 are distinct cDNAs, both of which contain full open reading frames that encode the same TA-GPCR (SEQ ID NO: 7). TA-GPCR shows high sequence similarity to known G protein coupled receptors, and likely plays a significant role in signal transduction. SEQ ID NO: 8 is a cDNA sequence containing a full open reading frame that encodes TA-PP2C (SEQ ID NO: 9). TA-PP2C shows high sequence similarity to known serine-threonine proteases, has a PP2C box, and likely functions to modulate signal transduction. SEQ ID NOS: 10 and 12 are cDNA sequences containing full open reading frames encoding a long (SEQ ID NO: 11) and a short (SEQ ID NO: 13) form of TA-NFKBH (SEQ ID NO: 11). TA-NFKBH has sequence similarity to known transcription factors, contains five Ankyrin repeats, and may play a part in gene regulation during T cell activation. SEQ ID NO: 14 is a cDNA sequence containing a full open reading frame that encodes TA-WDRP (SEQ ID NO: 15). TA-WDRP is a transducin-like protein with eleven WD repeats; based on its structural similarities with transducin, TLP is likely a G protein. SEQ ID NO: 16 is a cDNA sequence containing a full open reading frame that encodes TA-KRP (SEQ ID NO: 17). TA-KRP has three kelch repeat motifs and a POZ/BTB domain, and may be involved in G-protein signaling. SEQ ID NO: 18 is a cDNA sequence containing a full open reading frame that encodes TA-LLRP (SEQ ID NO: 19). TA-LLRP is a leucine-repeat rich protein. Diagrams of each of these proteins, showing their relative sizes and the positions of each of the domains noted above, are provided in FIG. 11.

The invention further relates to fragments and other derivatives of the above TCAPs. Nucleic acids encoding such fragments or derivatives are also within the scope of the invention. The invention provides TCAP-encoding genes (“TCAP genes”) and their encoded proteins of many different species. As used herein, “TCAP genes” includes cDNAs or other nucleic acids encoding a TCAP in whole or in part. The TCAP genes of the invention include human and related genes (homologs) in other species. In specific embodiments, the TCAP genes and proteins are from vertebrates, or more particularly, mammals. In a preferred embodiment of the invention, the TCAP genes and proteins are of human origin. Production of the foregoing proteins and derivatives, e.g., by recombinant methods, is provided.

The invention also relates to TCAP derivatives of the invention that are functionally active, i.e., they are capable of displaying one or more known functional activities associated with a full-length (wild-type) TCAPs. Such functional activities include but are not limited to activation of GTPases (TA-GAP), indirect activation of membrane-bound enzymes or ion channels (TA-GPCR); transcriptional activation (KBTF); phosphatase activity (TA-PP2C); GTPase activity and the ability to interact with GPCRs (TA-TCP); antigenicity (i.e., the ability to bind, or compete with a TCAP for binding, to an anti-TCAP antibody; immunogenicity (ability to generate an antibody which binds to a TCAP); ability to bind, or compete with TCAP for binding, to an TCAP-domain-containing protein or other ligand.

The invention further relates to fragments, and derivatives thereof, of TCAPs that comprise one or more domains of the TCAPs.

Antibodies to TCAPs, their derivatives, are additionally provided.

The present invention also relates to therapeutic and diagnostic methods and compositions based on TCAPs, TCAP nucleic acids and anti-TCAP antibodies. The invention provides for immunosuppression by administering compounds that inhibit or antagonize TCAP activity (e.g., antagonists of a TCAP; antisense molecules directed to the genes encoding a TCAP; antibodies to a TCAP).

Animal models, diagnostic methods and screening methods for predisposition to disorders are also provided by the invention.

The invention is illustrated by way of examples infra which disclose, inter alia, the cloning and characterization of the TCAPs (Section 6).

For clarity of disclosure, and not by way of limitation, the detailed description of the invention is divided into the subsections which follow.

### 5.1. ISOLATION OF THE TCAP GENES

The invention relates to the nucleotide sequences of nucleic acids. In a specific embodiment, the inventor relates to nucleic acids that encode a TCAP. In a more specific embodiment, the invention relates to nucleic acids that encode TA-GAP, TA-GPCR, TA-PP2C, TA-NFKBH, TA-KRP, TA-WDRP, or TA-LRRP. In further specific embodiments, TA-GAP nucleic acids comprise the cDNA sequences of SEQ ID NO: 1 or SEQ ID NO: 2, or the coding regions thereof, or nucleic acid encoding TA-GAP (e.g., a protein having the sequence of SEQ ID NO: 3 or SEQ ID NO: 4). In another specific embodiment, TA-GPCR nucleic acids comprise the cDNA sequences of SEQ ID NO: 5 or SEQ ID NO: 6, or the coding regions thereof, or nucleic acid encoding TA-GPCR, (e.g., a protein having the sequence of SEQ ID NO: 7). In another specific embodiment, TA-PP2C nucleic acids comprise the cDNA sequence of SEQ ID NO: 8, or the coding regions thereof, or nucleic acid encoding TA-PP2C (e.g., a protein having the sequence of SEQ ID NO: 9). In another specific embodiment, TA-NFKBH nucleic acids comprise the cDNA sequences of SEQ ID NO: 10 or SEQ ID NO: 12, or the coding regions thereof, or nucleic acid encoding TA-GPCR, (e.g., a protein having the sequence of SEQ ID NO: 11 or 13). In another specific embodiment, TA-WDRP nucleic acids comprise the cDNA sequence of SEQ ID NO: 14, or the coding regions thereof, or nucleic acid encoding TA-WDRP, (e.g., a protein having the sequence of SEQ ID NO: 15). In another specific embodiment, TA-KRP nucleic acids comprise the cDNA sequences of SEQ ID NO: 16, or the coding regions thereof, or nucleic acid encoding TA-KRP, (e.g., a protein having the sequence of SEQ ID NO: 17). In another specific embodiment, TA-LRRP nucleic acids comprise the cDNA sequence of SEQ ID NO: 18, or the coding regions thereof, or nucleic acid encoding TA-LRRP, (e.g., a protein having the sequence of SEQ ID NO: 19).

The invention provides purified nucleic acids consisting of at least 10 nucleotides (i.e., a hybridizable portion) of a nucleotide sequence encoding a TCAP; in other embodiments, the nucleic acids consist of at least 10, 20, 50, 100, 150, or 200 contiguous nucleotides of a nucleotide sequence encoding a TCAP, or a full-length coding sequence. In another embodiment, the nucleic acids are smaller than 35, 200 or 500 nucleotides in length. Nucleic acids can be single or double stranded. In another embodiment, the nucleic acids comprise a sequence of at least 10 nucleotides that encode a fragment of a TCAP, wherein the fragment of the TCAP displays one or more functional activities of the TCAP, or contains a functional domain or motif of the TCAP. In no event, however, does the invention provide for a contiguous nucleic acid sequence present in the GenBank search results provided in the Examples in Section 6.

The invention also relates to nucleic acids hybridizable to or complementary to the foregoing sequences. In specific aspects, nucleic acids are provided which comprise a sequence complementary to at least 10, 25, 50, 100, or 200 nucleotides or the entire coding region of a gene encoding a TCAP, or the reverse complement (antisense) of any of these sequences. In a specific embodiment, a nucleic acid which is hybridizable to a TA-GAP nucleic acid (e.g., having part or the whole of sequence SEQ ID NO: 1 or SEQ ID NO: 2, or the complement thereof), or to a nucleic acid encoding a TA-GAP derivative, under conditions of low stringency is provided. In another specific embodiment, a nucleic acid which is hybridizable to a TA-GPCR nucleic acid (e.g., having part or the whole of SEQ ID NO: 5 or SEQ ID NO: 6, or the complement thereof), or to a nucleic acid encoding a TA-GPCR derivative, under conditions of low stringency is provided. In further specific embodiment, a nucleic acid which is hybridizable to a TA-PP2C nucleic acid (e.g., having part or the whole of SEQ ID NO: 8, or the complement thereof), or to a nucleic acid encoding a TA-PP2C derivative, under conditions of low stringency is provided. In a further specific embodiment, a nucleic acid which is hybridizable to a TA-NFKBH nucleic acid (e.g., having part or the whole of SEQ ID NO: 10 or 12, or the complement thereof), or to a nucleic acid encoding a TA-NFKBH derivative, under conditions of low stringency is provided. In another specific embodiment, a nucleic acid which is hybridizable to a TA-WDRP nucleic acid (e.g., having part or the whole of SEQ ID NO: 14, or the complement thereof), or to a nucleic acid encoding a TA-WDRP derivative, under conditions of low stringency is provided. In yet a further specific embodiment, a nucleic acid which is hybridizable to a TA-KRP nucleic acid (e.g., having part or the whole of SEQ ID NO: 16, or the complement thereof), or to a nucleic acid encoding a TA-KRP derivative, under conditions of low stringency is provided. In yet a further specific embodiment, a nucleic acid which is hybridizable to a TA-LRRP nucleic acid (e.g., having part or the whole of SEQ ID NO: 18, or the complement thereof), or to a nucleic acid encoding a TA-LRRP derivative, under conditions of low stringency is provided.

By way of example and not limitation, procedures using such conditions of low stringency are as follows (see also Shilo and Weinberg, Proc. Natl. Acad. Sci. U.S.A. 78:6789-6792 (1981)): Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg g/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and reexposed to film. Other conditions of low stringency which may be used are well known in the art (e.g., as employed for cross-species hybridizations).

In another specific embodiment, a nucleic acid hybridizable to a nucleic acid encoding a TCAP, or its inverse complement, under conditions of high stringency is provided. By way of example and not limitation, procedures using such conditions of high stringency are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency that may be used are well known in the art.

In another specific embodiment, a nucleic acid that is hybridizable to a nucleic acid encoding a TCAP under conditions of high stringency is provided (see, e.g., Section 5.1).

Nucleic acids hybridizable to the complement of the above-mentioned sequences are also provided.

The above-mentioned nucleic acids preferably also encode a protein displaying one or more functional activities of a TCAP or a domain or motif thereof.

Nucleic acids encoding derivatives of TCAPs (see Sections 5.6 and 5.6.1), and antisense nucleic acids to genes encoding TCAPs (see Section 5.7.3.1.1) are additionally provided. As is readily apparent, as used herein, a nucleic acid encoding a “fragment” or “portion” of a TCAP shall be construed as referring to a nucleic acid encoding only the recited fragment or portion of the specific TCAP and not the other contiguous portions of the specific TCAP protein as a continuous sequence.

Fragments of nucleic acids encoding the TCAPs described above, which comprise regions conserved between (i.e., having homology or identity to) other TCAP-encoding nucleic acids of the same or different species, are also provided. Nucleic acids encoding one or more domains of a specific TCAP are provided.

Fragments or derivatives of TCAP nucleic acids that hybridize specifically to one or more TCAP nucleic acids, and thus can be used as hybridization probes in hybridization assays to detect T cell activation or a lack thereof are also provided. In such embodiments, oligonucleotides of at least 10, 15, 20, 25 or 30 nucleotides are provided. In specific embodiments, oligonucleotides, preferably oligodeoxynucleotides, in the range of 10-100, 15-80, or 40-70 nucleotides are provided as hybridization probes.

As a non-limiting example of suitable TCAP nucleotide sequences useful for hybridization probes or primers for PCR, sequences may be selected from the following SEQ ID NO: 1 cDNA nucleotides, or the complements thereof (nn.sub.x-nn.sub.y denotes from about nucleotide number x to about nucleotide number y)): nn.sub.1-nn.sub.10; nn.sub.10-nn.sub.20; nn.sub.20-nn.sub.30; nn.sub.30-nn.sub.40; nn.sub.40-nn.sub.50; nn.sub.50-nn.sub.60; nn.sub.60-nn.sub.70; nn.sub.70-nn.sub.80; nn.sub.80-nn.sub.90; nn.sub.90-nn.sub.100; nn.sub.100-nn.sub.110; nn.sub.110-nn.sub.120; nn.sub.120 -nn.sub.130; nn.sub.130-nn.sub.140; nn.sub.140-nn.sub.150; nn.sub.150-nn.sub.160; nn.sub.160-nn.sub.170; nn.sub.170-nn.sub.180; nn.sub.180-nn.sub.190; nn.sub.190 -nn.sub.200; nn.sub.200-nn.sub.210; nn.sub.210-nn.sub.220; nn.sub.220-nn.sub.230; nn.sub.230-nn.sub.240; nn.sub.240-nn.sub.250; nn.sub.250-nn.sub.260; nn.sub.260 -nn.sub.270; nn.sub.270-nn.sub.280; nn.sub.280-nn.sub.290; nn.sub.290-nn.sub.300; nn.sub.300-nn.sub.310; nn.sub.310-nn.sub.320; nn.sub.320-nn.sub.330; nn.sub.330 -nn.sub.340; nn.sub.340-nn.sub.350; nn.sub.350-nn.sub.360; nn.sub.360-nn.sub.370; nn.sub.370-nn.sub.380; nn.sub.380-nn.sub.390; nn.sub.390-nn.sub.400; nn.sub.400 -nn.sub.410; nn.sub.410-nn.sub.420; nn.sub.420-nn.sub.430; nn.sub.430-nn.sub.440; nn.sub.440-nn.sub.450; nn.sub.450-nn.sub.460; nn.sub.460-nn.sub.470; nn.sub.470 -nn.sub.480; nn.sub.480-nn.sub.490; nn.sub.490-nn.sub.500; nn.sub.500-nn.sub.510; nn.sub.510-nn.sub.520; nn.sub.520-nn.sub.530; nn.sub.530-nn.sub.540; nn.sub.540 -nn.sub.550; nn.sub.550-nn.sub.560; nn.sub.560-nn.sub.570; nn.sub.570-nn.sub.580; nn.sub.580-nn.sub.590; nn.sub.590-nn.sub.600; nn.sub.600-nn.sub.610; nn.sub.610 -nn.sub.620; nn.sub.620-nn.sub.630; nn.sub.630-nn.sub.640; nn.sub.640-nn.sub.650; nn.sub.650-nn.sub.660; nn.sub.660-nn.sub.670; nn.sub.670-nn.sub.680; nn.sub.680 -nn.sub.690; nn.sub.690-nn.sub.700; nn.sub.700-nn.sub.710; nn.sub.710-nn.sub.720; nn.sub.720-nn.sub.730; nn.sub.730-nn.sub.740; nn.sub.740-nn.sub.750; nn.sub.750 -nn.sub.760; nn.sub.760-nn.sub.770; nn.sub.770-nn.sub.780; nn.sub.780-nn.sub.790; nn.sub.790-nn.sub.800; nn.sub.800-nn.sub.810; nn.sub.810-nn.sub.820; nn.sub.820 -nn.sub.830; nn.sub.830-nn.sub.840; nn.sub.840-nn.sub.850; nn.sub.850-nn.sub.860; nn.sub.860-nn.sub.870; nn.sub.870-nn.sub.880; nn.sub.880-nn.sub.890; nn.sub.890 -nn.sub.900; nn.sub.900-nn.sub.910; nn.sub.910-nn.sub.920; nn.sub.920-nn.sub.930; nn.sub.930-nn.sub.940; nn.sub.940-nn.sub.950; nn.sub.950-nn.sub.960; nn.sub.960 -nn.sub.970; nn.sub.970-nn.sub.980; nn.sub.980-nn.sub.990; nn.sub.990-nn.sub.1000; nn.sub.1000-nn.sub.1010; nn.sub.1010-nn.sub.1020; nn.sub.1020-nn.sub.1030; nn.sub.1030-nn.sub.1040; nn.sub.1040-nn.sub.1050; nn.sub.1050-nn.sub.1060; nn.sub.1060-nn.sub.1070; nn.sub.1070-nn.sub.1080; nn.sub.1080-nn.sub.1090; nn.sub.1090-nn.sub.1100; nn.sub.1100-nn.sub.1110; nn.sub.1110-nn.sub.1120; nn.sub.1120-nn.sub.1130; nn.sub.1130-nn.sub.1140; nn.sub.1140-nn.sub.1150; nn.sub.1150-nn.sub.1160; nn.sub.1160-nn.sub.1170; nn.sub.1170-nn.sub.1180; nn.sub.1180-nn.sub.1190; nn.sub.1190-nn.sub.1200; nn.sub.1200-nn.sub.1210; nn.sub.1210-nn.sub.1220; nn.sub.1220-nn.sub.1230; nn.sub.1230-nn.sub.1240; nn.sub.1240-nn.sub.1250; nn.sub.1250-nn.sub.1260; nn.sub.1260-nn.sub.1270; nn.sub.1270-nn.sub.1280; nn.sub.1280-nn.sub.1290; nn.sub.1290-nn.sub.1300; nn.sub.1300-nn.sub.1310; nn.sub.1310-nn.sub.1320; nn.sub.1320-nn.sub.1330; nn.sub.1330-nn.sub.1340; nn.sub.1340-nn.sub.1350; nn.sub.1350-nn.sub.1360; nn.sub.1360-nn.sub.1370; nn.sub.1370-nn.sub.1380; nn.sub.1380-nn.sub.1390; nn.sub.1390-nn.sub.1400; nn.sub.1400-nn.sub.1410; nn.sub.1410-nn.sub.1420; nn.sub.1420-nn.sub.1430; nn.sub.1430-nn.sub.1440; nn.sub.1440-nn.sub.1450; nn.sub.1450-nn.sub.1460; nn.sub.1460-nn.sub.1470; nn.sub.1470-nn.sub.1480; nn.sub.1480-nn.sub.1490; nn.sub.1490-nn.sub.1500; nn.sub.1500-nn.sub.1510; nn.sub.1510-nn.sub.1520; nn.sub.1520-nn.sub.1530; nn.sub.1530-nn.sub.1540; nn.sub.1540-nn.sub.1550; nn.sub.1550-nn.sub.1560; nn.sub.1560-nn.sub.1570; nn.sub.1570-nn.sub.1580; nn.sub.1580-nn.sub.1590; nn.sub.1590-nn.sub.1600; nn.sub.1600-nn.sub.1610; nn.sub.1610-nn.sub.1620; nn.sub.1620-nn.sub.1630; nn.sub.1630-nn.sub.1640; nn.sub.1640-nn.sub.1650; nn.sub.1650-nn.sub.1660; nn.sub.1660-nn.sub.1670; nn.sub.1670-nn.sub.1680; nn.sub.1680-nn.sub.1690; nn.sub.1690-nn.sub.1700; nn.sub.1700-nn.sub.1710; nn.sub.1710-nn.sub.1720; nn.sub.1720-nn.sub.1730; nn.sub.1730-nn.sub.1740; nn.sub.1740-nn.sub.1750; nn.sub.1750-nn.sub.1760; nn.sub.1760-nn.sub.1770; nn.sub.1770-nn.sub.1780; nn.sub.1780-nn.sub.1790; nn.sub.1790-nn.sub.1800; nn.sub.1800-nn.sub.1810; nn.sub.1810-nn.sub.1820; nn.sub.1820-nn.sub.1830; nn.sub.1830-nn.sub.1840; nn.sub.1840-nn.sub.1850; nn.sub.1850-nn.sub.1860; nn.sub.1860-nn.sub.1870; nn.sub.1870-nn.sub.1880; nn.sub.1880-nn.sub.1890; nn.sub.1890-nn.sub.1900; nn.sub.1900-nn.sub.1910; nn.sub.1910-nn.sub.1920; nn.sub.1920-nn.sub.1930; nn.sub.1930-nn.sub.1940; nn.sub.1940-nn.sub.1950; nn.sub.1950-nn.sub.1960; nn.sub.1960-nn.sub.1970; nn.sub.1970-nn.sub.1980; nn.sub.1980-nn.sub.1990; nn.sub.1990-nn.sub.2000; nn.sub.2000-nn.sub.2010; nn.sub.2010-nn.sub.2020; nn.sub.2020-nn.sub.2030; nn.sub.2030-nn.sub.2040; nn.sub.2040-nn.sub.2050; nn.sub.2050-nn.sub.2060; nn.sub.2060-nn.sub.2070; nn.sub.2070-nn.sub.2080; nn.sub.2080-nn.sub.2090; nn.sub.2090-nn.sub.2100; nn.sub.2100-nn.sub.2110; nn.sub.2110-nn.sub.2120; nn.sub.2120-nn.sub.2130; nn.sub.2130-nn.sub.2140; nn.sub.2140-nn.sub.2150; nn.sub.2150-nn.sub.2160; nn.sub.2160-nn.sub.2170; nn.sub.2170-nn.sub.2180; nn.sub.2180-nn.sub.2190; nn.sub.2190-nn.sub.2200; nn.sub.2200-nn.sub.2210; nn.sub.2210-nn.sub.2220; nn.sub.2220-nn.sub.2230; nn.sub.2230-nn.sub.2240; nn.sub.2240-nn.sub.2250; nn.sub.2250-nn.sub.2260; nn.sub.2260-nn.sub.2270; nn.sub.2270-nn.sub.2280; nn.sub.2280-nn.sub.2290; nn.sub.2290-nn.sub.2300; nn.sub.2300-nn.sub.2310; nn.sub.2310-nn.sub.2320; nn.sub.2320-nn.sub.2330; nn.sub.2330-nn.sub.2340; nn.sub.2340-nn.sub.2350; nn.sub.2350-nn.sub.2360; nn.sub.2360-nn.sub.2370; nn.sub.2370-nn.sub.2380; nn.sub.2380-nn.sub.2390; nn.sub.2390-nn.sub.2400; nn.sub.2400-nn.sub.2410; nn.sub.2410-nn.sub.2420; nn.sub.2420-nn.sub.2430; nn.sub.2430-nn.sub.2440; nn.sub.2440-nn.sub.2450; nn.sub.2450-nn.sub.2460; nn.sub.2460-nn.sub.2470; nn.sub.2470-nn.sub.2480; nn.sub.2480-nn.sub.2490; nn.sub.2490-nn.sub.2500; nn.sub.2500-nn.sub.2510; nn.sub.2510-nn.sub.2520; nn.sub.2520-nn.sub.2530; nn.sub.2530-nn.sub.2540; nn.sub.2540-nn.sub.2550; nn.sub.2550-nn.sub.2560; nn.sub.2560-nn.sub.2570; nn.sub.2570-nn.sub.2580; nn.sub.2580-nn.sub.2590; nn.sub.2590-nn.sub.2600; nn.sub.2600-nn.sub.2610; nn.sub.2610-nn.sub.2620; nn.sub.2620-nn.sub.2630; nn.sub.2630-nn.sub.2640; nn.sub.2640-nn.sub.2650; nn.sub.2650-nn.sub.2660; nn.sub.2660-nn.sub.2670; nn.sub.2670-nn.sub.2680; nn.sub.2680-nn.sub.2690; nn.sub.2690-nn.sub.2700; nn.sub.2700-nn.sub.2710; nn.sub.2710-nn.sub.2720; nn.sub.2720-nn.sub.2730; nn.sub.2730-nn.sub.2740; nn.sub.2740-nn.sub.2750; nn.sub.2750-nn.sub.2760; nn.sub.2760-nn.sub.2770; nn.sub.2770-nn.sub.2780; nn.sub.2780-nn.sub.2790; nn.sub.2790-nn.sub.2800; nn.sub.2800-nn.sub.2810; nn.sub.2810-nn.sub.2820; nn.sub.2820-nn.sub.2830; nn.sub.2830-nn.sub.2840; nn.sub.2840-nn.sub.2850; nn.sub.2850-nn.sub.2860; nn.sub.2860-nn.sub.2870; nn.sub.2870-nn.sub.2880; nn.sub.2880-nn.sub.2890; nn.sub.2890-nn.sub.2900; nn.sub.2900-nn.sub.2910; nn.sub.2910-nn.sub.2920; nn.sub.2920-nn.sub.2930; nn.sub.2930-nn.sub.2940; nn.sub.2940-nn.sub.2950; nn.sub.2950-nn.sub.2960; nn.sub.2960-nn.sub.2970; nn.sub.2970-nn.sub.2980; nn.sub.2980-nn.sub.2990; nn.sub.2990-nn.sub.3000; nn.sub.3000-nn.sub.3010; nn.sub.3010-nn.sub.3020; nn.sub.3020-nn.sub.3030; nn.sub.3030-nn.sub.3040; nn.sub.3040-nn.sub.3050; nn.sub.3050-nn.sub.3060; nn.sub.3060-nn.sub.3070; nn.sub.3070-nn.sub.3080; nn.sub.3080-nn.sub.3090; nn.sub.3090-nn.sub.3100; nn.sub.3100-nn.sub.3110; nn.sub.3110-nn.sub.3120; nn.sub.3120-nn.sub.3130; nn.sub.3130-nn.sub.3140; nn.sub.3140-nn.sub.3150; nn.sub.3150-nn.sub.3160; nn.sub.3160-nn.sub.3170; nn.sub.3170-nn.sub.3180; nn.sub.3180-nn.sub.3190; nn.sub.3190-nn.sub.3200; nn.sub.3200-nn.sub.3210; nn.sub.3208-nn.sub.3218.

Sequences suitable for hybridization to SEQ ID NO: 3, SEQ ID NO: 5, SEQ ID NO: 6, SEQ ID NO: 8, SEQ ID NO: 10, SEQ ID NO: 12, SEQ ID NO: 14, SEQ ID NO: 16 or SEQ ID NO: 18 or their complements may be obtained in similar fashion.

The invention also provides nucleic acids comprising nucleotide sequences of at least 30, 50, 60, 70, 90, 95 or 99% homologous to a nucleotide sequence of a TCAP gene or a portion thereof. “Homologous” means that in various embodiments, the aligned first nucleotide sequence has preferably at least 30% or 50%, more preferably 60% or 70%, even more preferably at least 80% or 90%, and even more preferably at least 95% identity to a second nucleotide sequence over a nucleotide sequence length equal to the shorter of the two sequences, plus any introduced gaps. When the alignment is done by a computer homology program known in the art, such as BLAST (blastn), the percent homology is calculated by dividing the number of nucleotides in the TCAP-encoding nucleic acid sequence or fragment thereof exactly matching the nucleotide at the same position in the aligned sequence by the length of the alignment in nucleotides, including introduced gaps, where introduced gaps count as mismatches.

Specific embodiments for the cloning of a gene encoding a TCAP, presented as a particular example but not by way of limitation, follows:

For expression cloning (a technique commonly known in the art), an expression library is constructed by methods known in the art. For example, mRNA (e.g., human) is isolated, cDNA is made and ligated into an expression vector (e.g., a bacteriophage derivative) such that it is capable of being expressed by the host cell into which it is then introduced. Various screening assays can then be used to select for the expressed TCAP product. In one embodiment, anti-TA-GAP antibodies can be used for selection. In another embodiment, anti-TA-GPCR antibodies can be used for selection. In another embodiment, anti-TA-NFKBH antibodies can be used for selection. In yet another embodiment, anti-TA-KRP antibodies can be used for selection. In yet a further embodiment, anti-TA -PP2C antibodies can be used for selection. In another embodiment, anti-TA-LRRP antibodies can be used for selection. In yet another embodiment, anti-TA-LRRP antibodies can be used for selection.

In another embodiment of the invention, polymerase chain reaction (PCR) is used to amplify the desired sequence in a genomic or cDNA library, prior to selection. Oligonucleotide primers representing known TCAP-encoding sequences can be used as primers in PCR. In a preferred aspect, the oligonucleotide primers represent at least part of the conserved segments of strong homology between TCAP-encoding genes of different species, for example transmembrane domains, WD repeat domains, kelch motifs, β propellers, Ank-repeat domains, leucine-rich regions and ligand-binding domains. The synthetic oligonucleotides may be utilized as primers to amplify by PCR sequences from RNA or DNA, preferably a cDNA library, of potential interest. Alternatively, one can synthesize degenerate primers for use in the PCR reactions.

In PCR according to the invention, the nucleic acid being amplified can include RNA or DNA, for example, mRNA, cDNA or genomic DNA from any eukaryotic species. PCR can be carried out, e.g., by use of a Perkin-Elmer Cetus thermal cycler and Taq polymerase. It is also possible to vary the stringency of hybridization conditions used in priming the PCR reactions, to allow for greater or lesser degrees of nucleotide sequence similarity between a known TCAP nucleotide sequence and a nucleic acid homolog being isolated. For cross-species hybridization, low stringency conditions are preferred. For same-species hybridization, moderately stringent conditions are preferred. After successful amplification of a segment of a TCAP gene homolog, that segment may be cloned, sequenced, and utilized as a probe to isolate a complete cDNA or genomic clone. This, in turn, will permit the determination of the gene's complete nucleotide sequence, the analysis of its expression, and the production of its protein product for functional analysis, as described infra. In this fashion, additional genes encoding TCAPs and TCAP may be identified.

The above recited methods are not meant to limit the following general description of methods by which clones of genes encoding TCAPs may be obtained.

Any eukaryotic cell potentially can serve as the nucleic acid source for the molecular cloning of a TCAP-encoding gene. The nucleic acid sequences encoding TCAPs can be isolated from vertebrate, mammalian, human, porcine, bovine, feline, avian, equine, canine, as well as additional primate sources. The DNA may be obtained by standard procedures known in the art from cloned DNA (e.g., a DNA “library”), by chemical synthesis, by cDNA cloning, or by the cloning of genomic DNA, or fragments thereof, purified from the desired cell. (See, for example, Sambrook et al., Molecular Cloning, A Laboratory Manual, 2d. ed., Cold Spring Harbor Laboratory Press, Cold Spring Harbor, New York (1989); Glover, D. M. (ed.), DNA Cloning: A Practical Approach, MRL Press, Ltd., Oxford, U.K. Vol. I, II (1985)). Clones derived from genomic DNA may contain regulatory and intron DNA regions in addition to coding regions; clones derived from cDNA will contain only exon sequences. Whatever the source, the gene should be cloned into a suitable vector for propagation of the gene.

In the cloning of the gene from genomic DNA, DNA fragments are generated, some of which will encode the desired gene. The DNA may be cleaved at specific sites using various restriction enzymes. Alternatively, one may use DNase in the presence of manganese to fragment the DNA, or the DNA can be physically sheared, as for example, by sonication. The linear DNA fragments can then be separated according to size by standard techniques, including but not limited to, agarose and polyacrylamide gel electrophoresis and column chromatography.

Once the DNA fragments are generated, identification of the specific DNA fragment containing the desired gene may be accomplished in a number of ways. For example, if an TCAP gene (of any species) or its specific RNA, or a derivative thereof (see Section 5.6) is available and can be purified and labeled, the generated DNA fragments may be screened by nucleic acid hybridization to the labeled probe (Benton and Davis, Science 196:180 (1977); Grunstein And Hogness, Proc. Natl. Acad. Sci. U.S.A. 72:3961 (1975). Those DNA fragments with substantial homology to the probe will hybridize. It is also possible to identify the appropriate fragment by restriction enzyme digestion(s) and comparison of fragment sizes with those expected according to a known restriction map if such is available. Further selection can be carried out on the basis of the properties of the gene.

Alternatively, the presence of the gene may be detected by assays based on the physical, chemical, or immunological properties of its expressed product. For example, cDNA clones, or DNA clones that hybrid-select the proper mRNAs, can be selected that produce a protein having e.g., similar or identical electrophoretic migration, isoelectric focusing behavior, proteolytic digestion maps, kinase activity, inhibition of cell proliferation activity, substrate binding activity, or antigenic properties as known for a specific TCAP. If an antibody to a particular TCAP is available, that TCAP may be identified by binding of labeled antibody to the clone(s) putatively producing the TCAP in an ELISA (enzyme-linked immunosorbent assay)-type procedure.

A TCAP gene can also be identified by mRNA selection by nucleic acid hybridization followed by in vitro translation. In this procedure, fragments are used to isolate complementary mRNAs by hybridization. Such DNA fragments may represent available, purified DNA of another species containing a gene encoding a TCAP. Immunoprecipitation analysis or functional assays (e.g., aggregation ability in vitro; binding to receptor; see infra) of the in vitro translation products of the isolated products of the isolated mRNAs identifies the mRNA and, therefore, the complementary DNA fragments that contain the desired sequences. In addition, specific mRNAs may be selected by adsorption of polysomes isolated from cells to immobilized antibodies specifically directed against a specific TCAP. A radiolabelled TCAP cDNA can be synthesized using the selected mRNA (from the adsorbed polysomes) as a template. The radiolabelled mRNA or cDNA may then be used as a probe to identify the TCAP DNA fragments from among other genomic DNA fragments.

Alternatives to isolating the TCAP genomic DNA include, but are not limited to, chemically synthesizing the gene sequence itself from a known sequence or making cDNA to the mRNA which encodes a TCAP. For example, RNA for cDNA cloning of TA-GPCR, TA-GAP, TA-NFKBH, TA-KRP, TA-PP2C, TA-WDRP or TA-LBRP can be isolated from cells that express TA-GPCR, TA-GAP, TA-NFKBH, TA-KRP, TA-PP2C, TA-WDRP or TA-LRRP. Other methods are possible and within the scope of the invention.

The identified and isolated gene can then be inserted into an appropriate cloning vector. A large number of vector-host systems known in the art may be used. Possible vectors include, but are not limited to, plasmids or modified viruses, but the vector system must be compatible with the host cell used. Such vectors include, but are not limited to, bacteriophages such as lambda derivatives, or plasmids such as pBR322 or pUC plasmid derivatives or the pBluescript vector (Stratagene). The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and TCAP-encoding gene may be modified by homopolymeric tailing. Recombinant molecules can be introduced into host cells via transformation, transfection, infection, electroporation, etc., so that many copies of the gene sequence are generated.

In an alternative method, the desired gene may be identified and isolated after insertion into a suitable cloning vector in a “shotgun” approach. Enrichment for the desired gene, for example, by size fractionization, can be done before insertion into the cloning vector.

In specific embodiments, transformation of host cells with recombinant DNA molecules that incorporate the isolated TCAP-encoding gene, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

It will be understood that the RNA sequence equivalent of the nucleotide sequences provided herein can be easily and routinely generated by the substitution of thymine (T) residues with uracil (U) residues.

The TCAP sequences provided by the instant invention include those nucleotide sequences encoding substantially the same amino acid sequences as found in native TCAP proteins, and those encoded amino acid sequences with functionally equivalent amino acids, as well as those encoding other TCAP derivatives, as described in Sections 5.6 and 5.6.1 infra for derivatives of the TCAPs described herein.

### 5.2. EXPRESSION OF GENES ENCODING TCAPS

The nucleotide sequence coding for a TCAP or a functionally active fragment or other derivative thereof (see Section 5.6), can be inserted into an appropriate expression vector, i.e., a vector which contains the necessary elements for the transcription and translation of the inserted protein-coding sequence. The necessary transcriptional and translational signals can also be supplied by the native TCAP gene and/or its flanking regions. A variety of host-vector systems may be utilized to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. In specific embodiments, the human TA-GPCR, TA-GAP, TA-NFKBH, TA-KRP, TA-PP2C, TA-WDRP or TA-LRRP gene is expressed, or a sequence encoding a functionally active portion of human TCAP encoded by one of these genes is expressed. In yet another embodiment, a fragment of a TCAP comprising a domain of the particular TCAP is expressed.

Any of the methods previously described for the insertion of DNA fragments into a vector may be used to construct expression vectors containing a chimeric gene consisting of appropriate transcriptional/translational control signals and the protein coding sequences. These methods may include in vitro recombinant DNA and synthetic techniques and in viva recombinants (genetic recombination). Expression of nucleic acid sequence encoding a TCAP or peptide fragment thereof may be regulated by a second nucleic acid sequence so that the TCAP or peptide fragment thereof is expressed in a host transformed with the recombinant DNA molecule. For example, expression of a TCAP may be controlled by any promoter/enhancer element known in the art. In a specific embodiment, the promoter is heterologous to (i.e., not a native promoter of) the specific TCAP-encoding gene. Promoters that may be used to control expression of TCAP-encoding genes include, but are not limited to, the SV40 early promoter region (Bemoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. U.S.A. 78:1441-1445 (1981)), the regulatory sequences of the metallothionein gene (Brinster et al., Nature 296:39-42 (1982)); prokaryotic expression vectors such as the β-lactamase promoter (Villa-Kamaroff et al., Proc. Natl. Acad. Sci. U.S.A. 75:3727-3731 (1978)), or the tat promoter (DeBoer et al., Proc. Natl. Acad. Sci. U.S.A. 80:21-25 (1983)); see also “Useful proteins from recombinant bacteria” in Scientific American, 242:74-94 (1980); plant expression vectors comprising the nopaline synthetase promoter region (Herrera-Estrella et al., Nature 303:209-213 (1983)) or the cauliflower mosaic virus 35S RNA promoter (Gardner et al., Nucl. Acids Res. 9:2871 (1981)), and the promoter of the photosynthetic enzyme ribulose biphosphate carboxylase (Herrera-Estrella et al., Nature 310:115-120 (1984)); promoter elements from yeast or other fungi such as the Gal4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region active in pancreatic acinar cells (Swift et al., Cell 38:639-646 (1984); Ornitz et al., Cold Spring Harbor Symp. Quant. Biol. 50:399-409 (1986); MacDonald, Hepatology 7:425-515 (1987)); insulin gene control region active in pancreatic beta cells (Hanahan, Nature 315:115-122 (1985)), immunoglobulin gene control region active in lymphoid cells (Grosschedl et al., Cell 38:647-658 (1984); Adames et al., Nature 318:533-538 (1985); Alexander et al., Mol. Cell. Biol. 7:1436-1444 (1987)), mouse mammary tumor virus control region active in testicular, breast, lymphoid and mast cells (Leder et al., Cell 45:485-495 (1986)), albumin gene control region active in liver (Pinkert et al., Genes and Devel. 1:268-276 (1987)), alpha-fetoprotein gene control region active in liver (Krumlauf et al., Mol. Cell. Biol. 5:1639-1648 (1985); Hammer et al., Science 235:53-58 (1987); alpha 1-antitrypsin gene control region active in the liver (Kelsey et al., Genes and Devel. 1:161-171 (1987)), beta-globin gene control region active in myeloid cells (Mogram et al., Nature 315:338-340 (1985); Kollias et al., Cell 46:89-94 (1986); myelin basic protein gene control region active in oligodendrocyte cells in the brain (Readhead et al., Cell 48:703-712 (1987)); myosin light chain-2 gene control region active in skeletal muscle (Sani, Nature 314:283-286 (1985)), and gonadotropic releasing hormone gene control region active in the hypothalamus (Mason et al., Science 234:1372-1378 (1986)).

In a specific embodiment, a vector is used that comprises a promoter operably linked to a TCAP-encoding nucleic acid, one or more origins of replication, and, optionally, one or more selectable markers (e.g., an antibiotic resistance gene).

In a specific embodiment, an expression construct is made by subcloning the coding sequence from a TCAP gene into the EcoRI restriction site of each of the three pGEX vectors (Glutathione S-Transferase expression vectors; Smith and Johnson, Gene 7:31-40 (1988)). This allows for the expression of the TCAP product from the subclone in the correct reading frame.

Expression vectors containing TCAP-encoding gene inserts can be identified by three general approaches: (a) nucleic acid hybridization, (b) presence or absence of “marker” gene functions, and (c) expression of inserted sequences. In the first approach, the presence of a TCAP-encoding gene inserted in an expression vector can be detected by nucleic acid hybridization using probes comprising sequences that are homologous to an inserted TCAP-encoding gene. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a TCAP gene in the vector. For example, if the TCAP-encoding gene is inserted within the marker gene sequence of the vector, recombinants containing the insert can be identified by the absence of the marker gene function. In the third approach, recombinant expression vectors can be identified by assaying the specific TCAP product expressed by the recombinant. Such assays can be based, for example, on the physical or functional properties of the specific TCAP in in vitro assay systems, e.g., kinase activity, binding with antibodies directed to the specific TCAP, or inhibition of cell function(s) performed, facilitated or affected by the specific TCAP.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors that can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered TCAP may be controlled. Furthermore, different host cells have characteristic and specific mechanisms for the translational and post-translational processing and modification (e.g., glycosylation, phosphorylation of proteins. Appropriate cell lines or host systems can be chosen to ensure the desired modification and processing of the foreign protein expressed.

For example, expression in a bacterial system can be used to produce an unglycosylated core protein product. Expression in yeast will produce a glycosylated product. Expression in mammalian cells can be used to ensure “native” glycosylation of a heterologous protein. Furthermore, different vector/host expression systems may affect processing reactions to different degrees.

In other specific embodiments, the specific TCAP, or fragment or derivative thereof, may be expressed as a fusion, or chimeric protein product, comprising the protein, fragment or derivative joined via a peptide bond to a protein sequence derived from a different protein. Such a chimeric product can be made by ligating the appropriate nucleic acid sequences encoding the desired amino acid sequences to each other by methods known in the art, in the proper coding frame, and expressing the chimeric product by methods commonly known in the art. In one embodiment, therefore, the invention includes an isolated nucleic acid comprising a sequence of at least 10 nucleotides encoding a chimeric TCAP, wherein the chimeric TCAP displays at least one of the functional activities of the wild-type TCAP, and at least one non-TCAP functional activity. Alternatively, such a chimeric product may be made by protein synthetic techniques, e.g., by use of a peptide synthesizer.

A person of skill in the art will appreciate that cDNA, genomic, and synthesized sequences can be cloned and expressed. One way to accomplish such expression is by transferring a TCAP gene, or a nucleic acid encoding a TCAP or fragment thereof, to cells in tissue culture. The expression of the transferred gene may be controlled by its native promoter, or can be controlled by a non-native promoter (see supra; Section 5.7.3.1, infra). In addition to transferring a nucleic acid comprising a nucleic acid sequence encoding an entire TCAP (i.e., equivalent to the wild type), the transferred nucleic acids can encode a functional portion of a particular TCAP, or a protein having at least 60% sequence identity to a TCAP disclosed herein, as compared over the length of the particular TCAP, or a polypeptide having at least 60% sequence similarity to a TCAP fragment, as compared over the length of the TCAP fragment. Introduction of the nucleic acid into the cell is accomplished by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. The expressed TCAPs or fragments thereof are isolated and purified as described below.

### 5.3. IDENTIFICATION AND PURIFICATION OF TCAP GENE PRODUCTS

In particular aspects, the invention provides amino acid sequences of TCAPs, preferably human TCAPs, and fragments and derivatives thereof which comprise an antigenic determinant (i.e., can be recognized by an antibody) or which are otherwise functionally active, as well as nucleic acid sequences encoding the foregoing. “Functionally active” TCAP material as used herein refers to that material displaying one or more known functional activities associated with a full-length (wild-type) TCAP, e.g., activities associated with G-coupled proteins (TA-GPCR), GTPase-inducing activity (TA-GAP), transcriptional activation activity (TA-NFKBH), protease activity (TA-PP2C) or transducin-like activity (i.e., the ability to transmit a signal between a GPCR and an effector protein (TA-WDRP); inhibition of these activities; binding to a substrate or binding partner of the proteins listed above; or antigenicity (binding to an antibody raised against one of these proteins), immunogenicity, and so forth.

In specific embodiments, the invention provides fragments of TA-GPCR, TA-GAP, TA-NFKBH, TA-KRP, TA-PP2C, TA-WDRP or TA-LRRP consisting of at least 6 amino acids, 10 amino acids, 50 amino acids, or of at least 75 amino acids. In other embodiments, the proteins comprise or consist essentially of an extracellular ligand-binding domain, transmembrane domain, or intracellular domain (TA-GPCR); Kelch repeats (TA-KRP); WD repeat domain (TA-WDRP); β-propeller (TA-KRP or TA-WDRP); GTP-binding domain (TA-GPCR; TA-GAP); rhoGAP domain (TA-GAP); Ankyrin repeat-containing domain (TA-NFKBH), leucine repeat-rich domain (TA-LLRP), POZ/BTB domain (TA-KRP), PP2C box (TA-PP2C), or any combination of the foregoing, of the above TCAPs. Fragments, or proteins comprising fragments, lacking some or all of the foregoing regions of the above TCAPs are also provided. Nucleic acids encoding the foregoing are also provided.

Once a recombinant that expresses the TCAP-encoding gene sequence, or part thereof, is identified, the resulting product can be analyzed. This analysis is achieved by assays based on the physical or functional properties of the product, including radioactive labeling of the product followed by analysis by gel electrophoresis, immunoassay, etc.

Once the particular TCAP, or fragment thereof, is identified, it may be isolated and purified by standard methods including chromatography (e.g., ion exchange, affinity, and sizing column chromatography), centrifugation, differential solubility, or by any other standard technique for the purification of proteins. The functional properties may be evaluated using any suitable assay (see Section 5.7).

Alternatively, once a TCAP produced by a recombinant is identified, the amino acid sequence of the protein can be deduced from the nucleotide sequence of the chimeric gene contained in the recombinant. As a result, the protein can be synthesized by standard chemical methods known in the art (e.g., see Hunkapiller et al., Nature 310:105-111 (1984)).

In another alternate embodiment, native TA-GPCR, TA-GAP, TA-NFKBH, TA-KRP, TA-PP2C, TA-WDRP or TA-LRRP proteins can be purified from natural sources, by standard methods such as those described above (e.g., immunoaffinity purification).

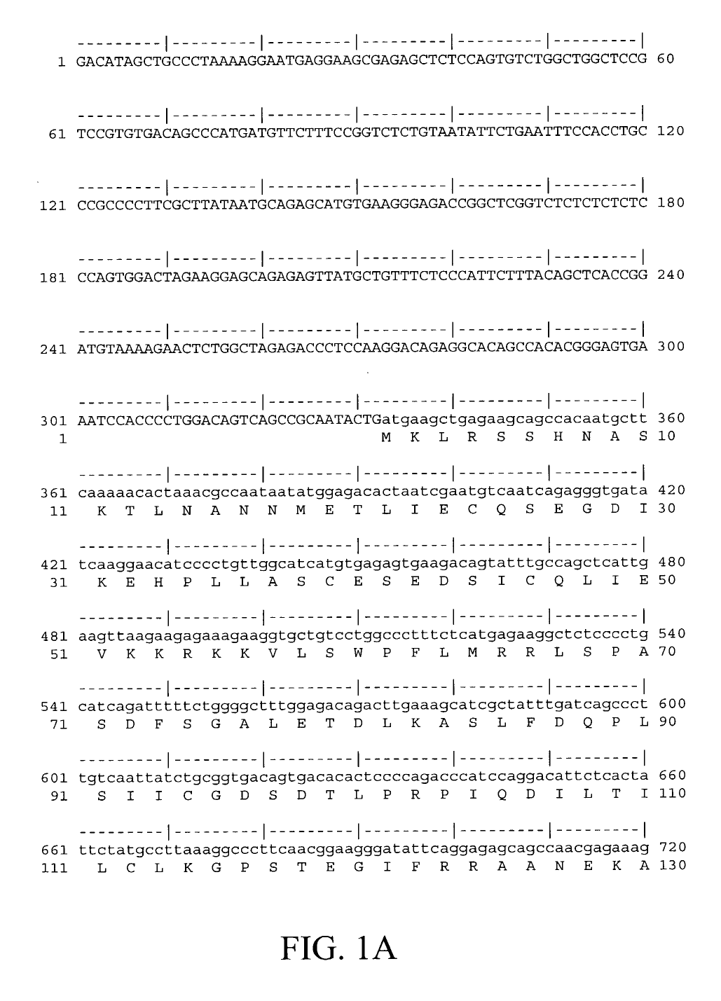

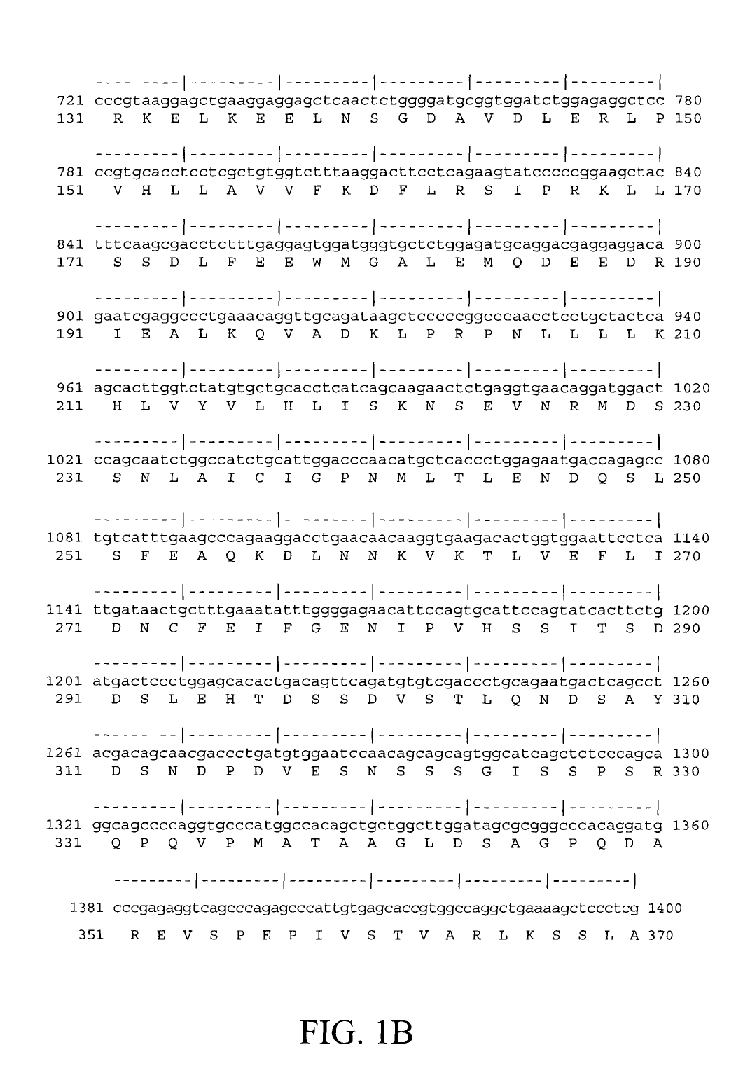

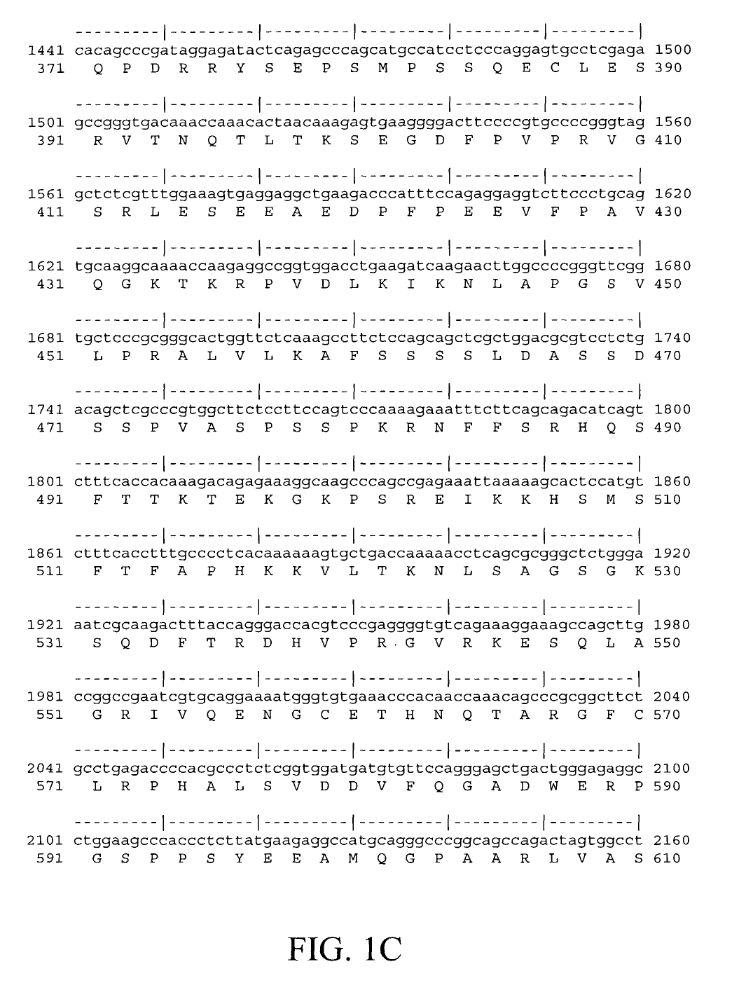

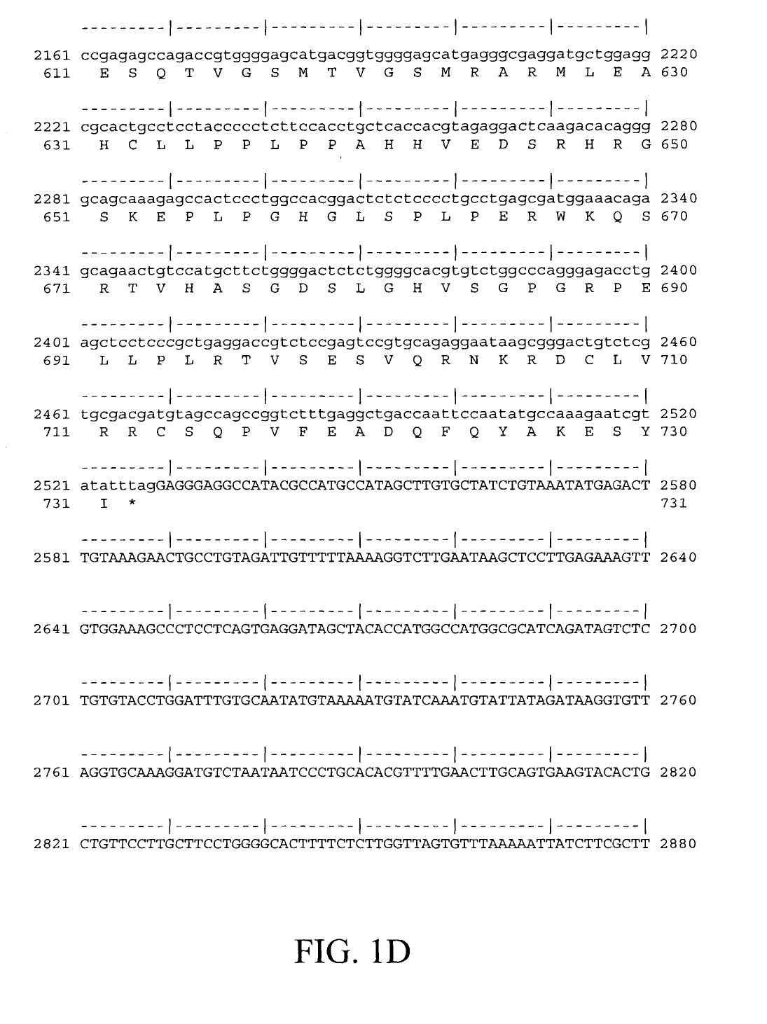

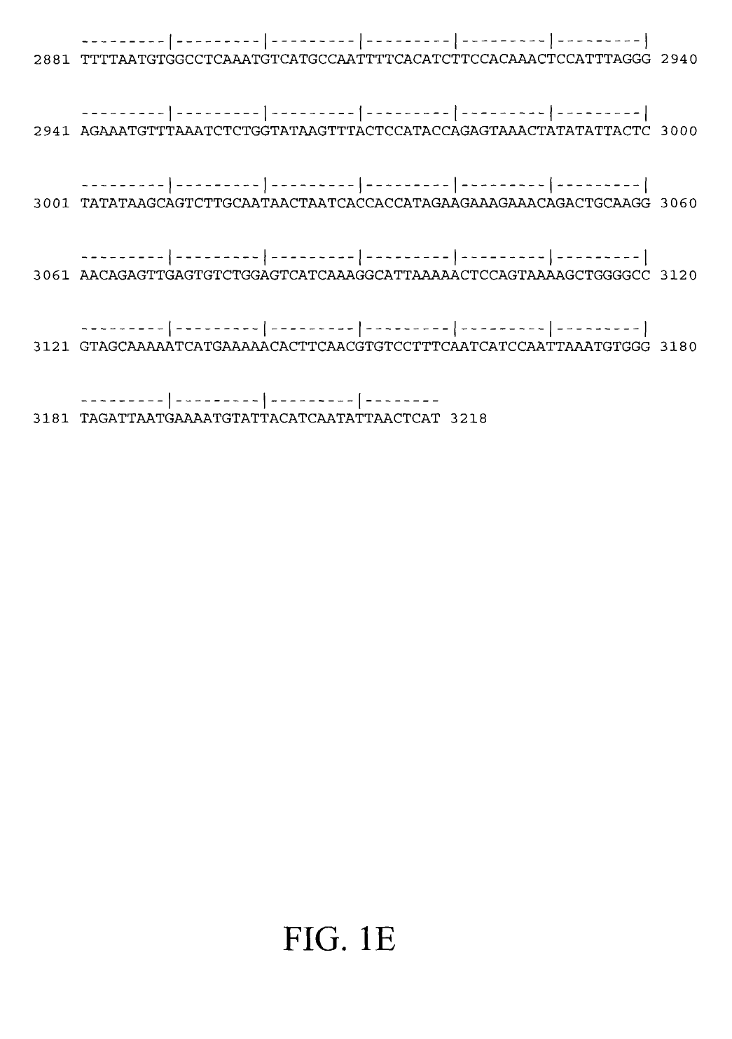

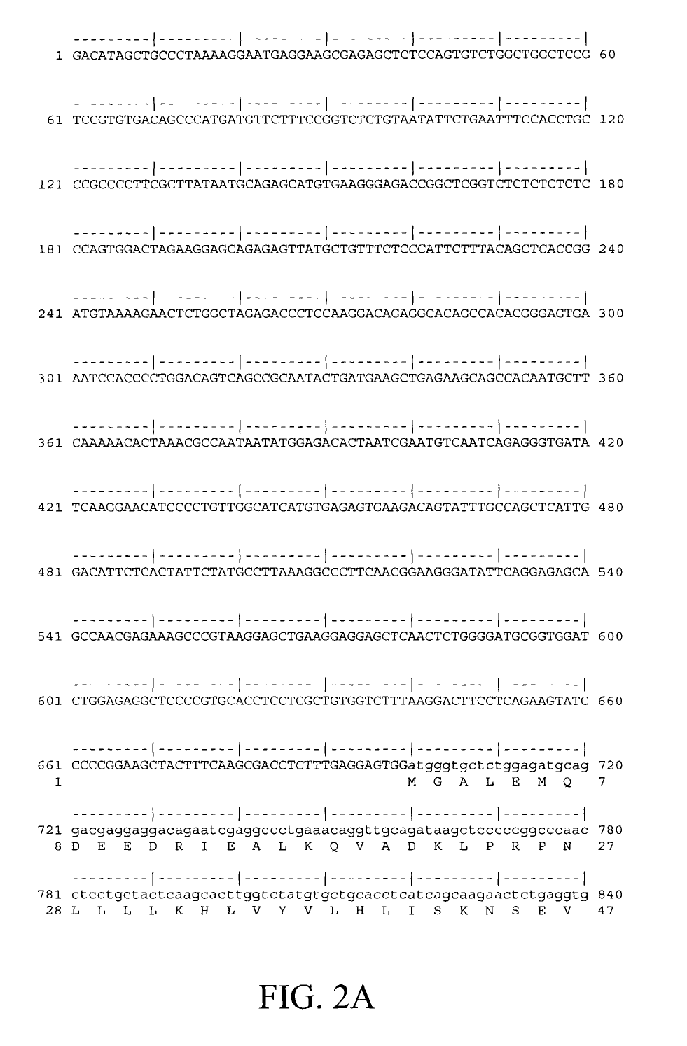

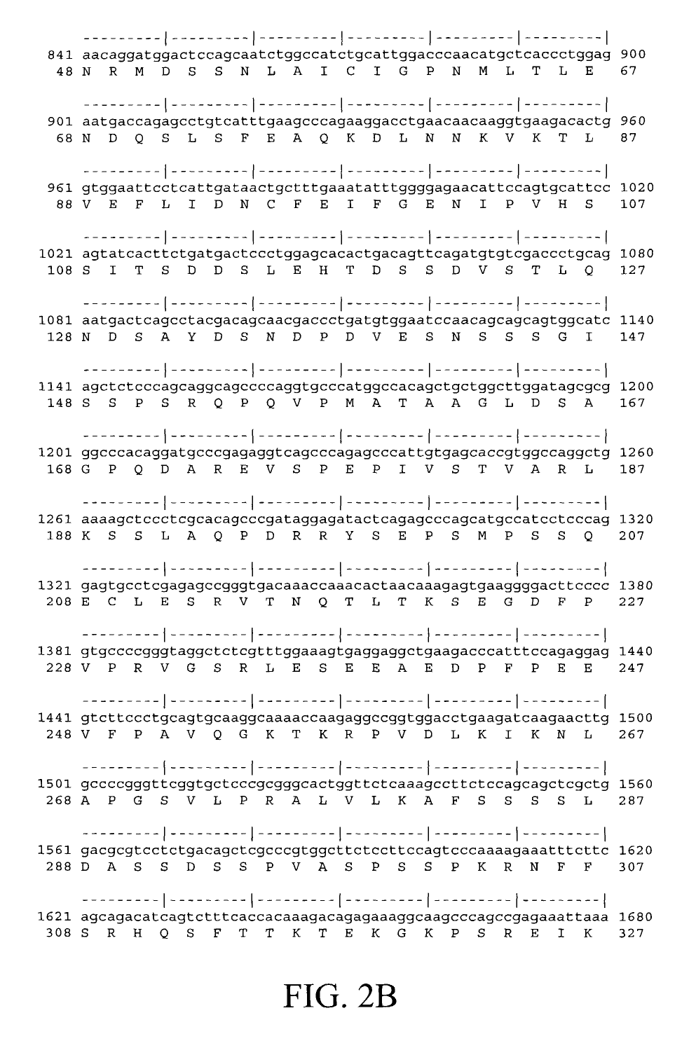

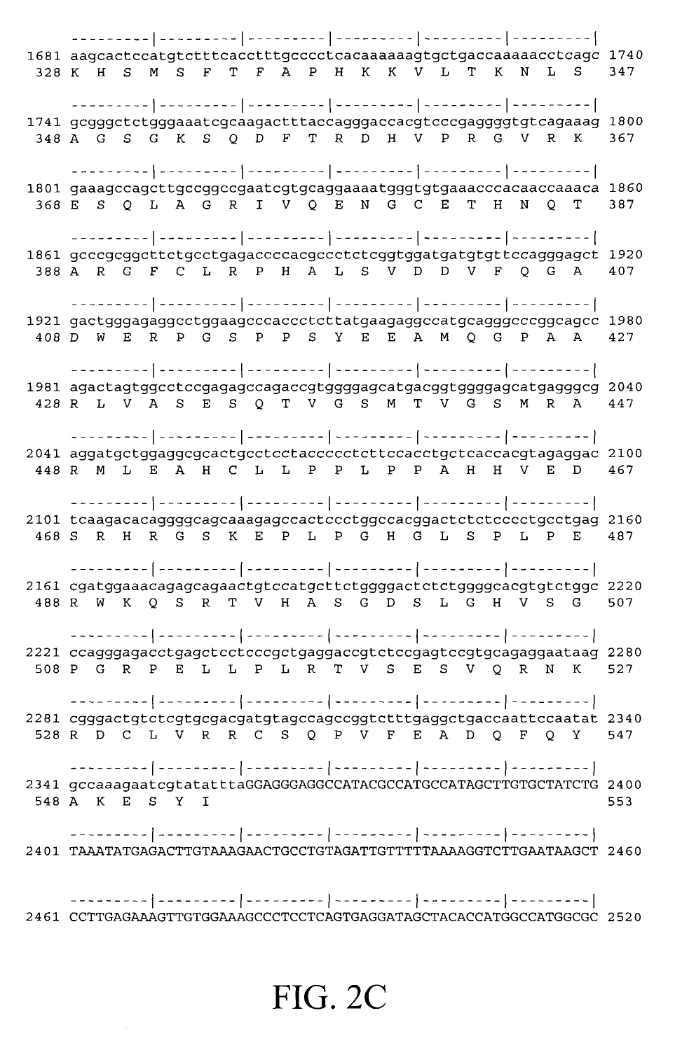

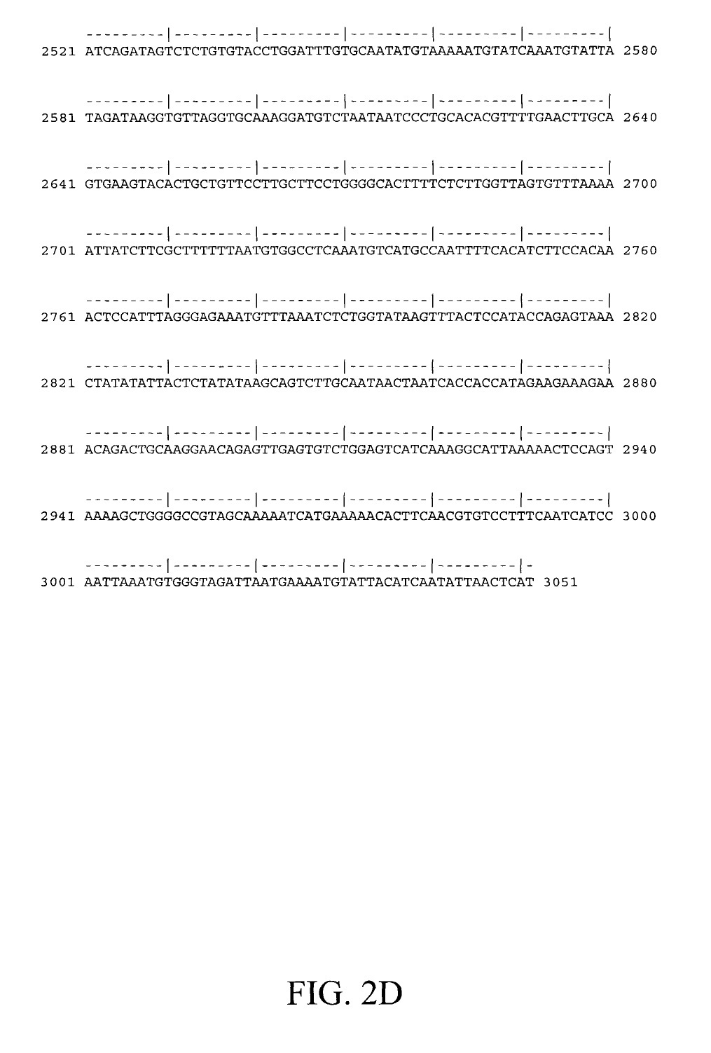

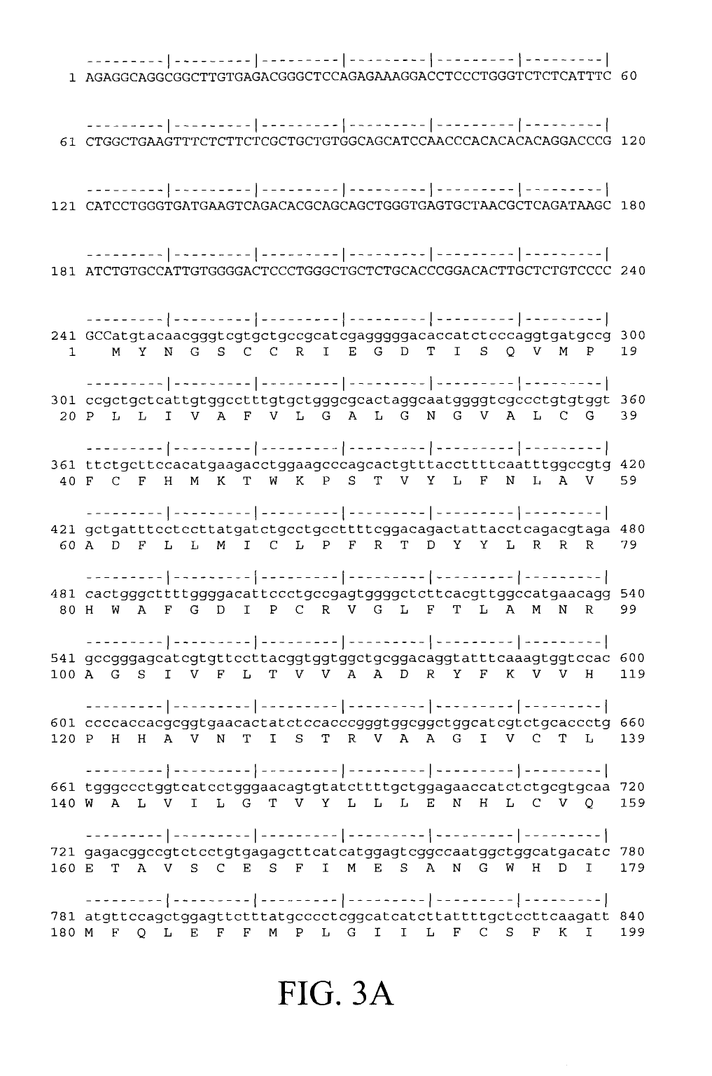

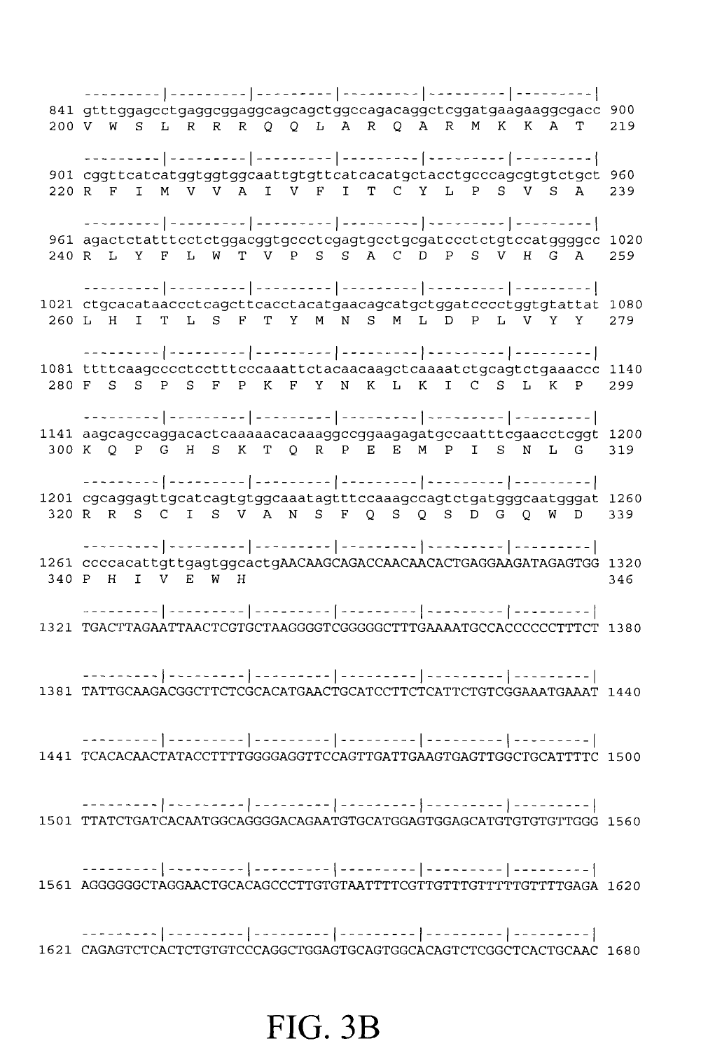

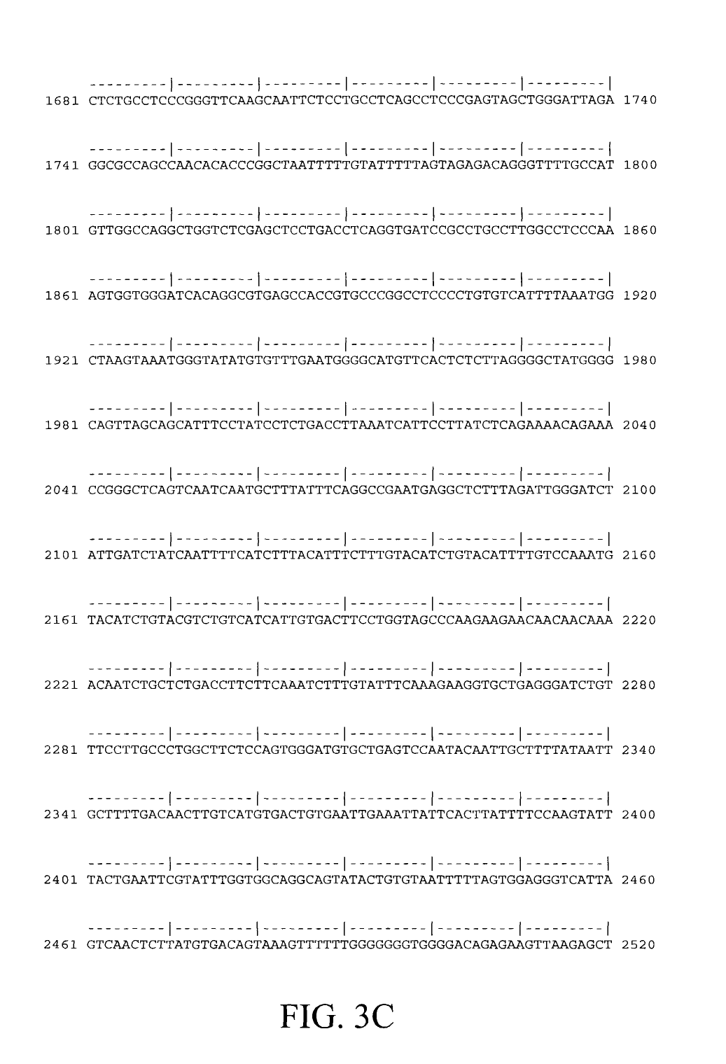

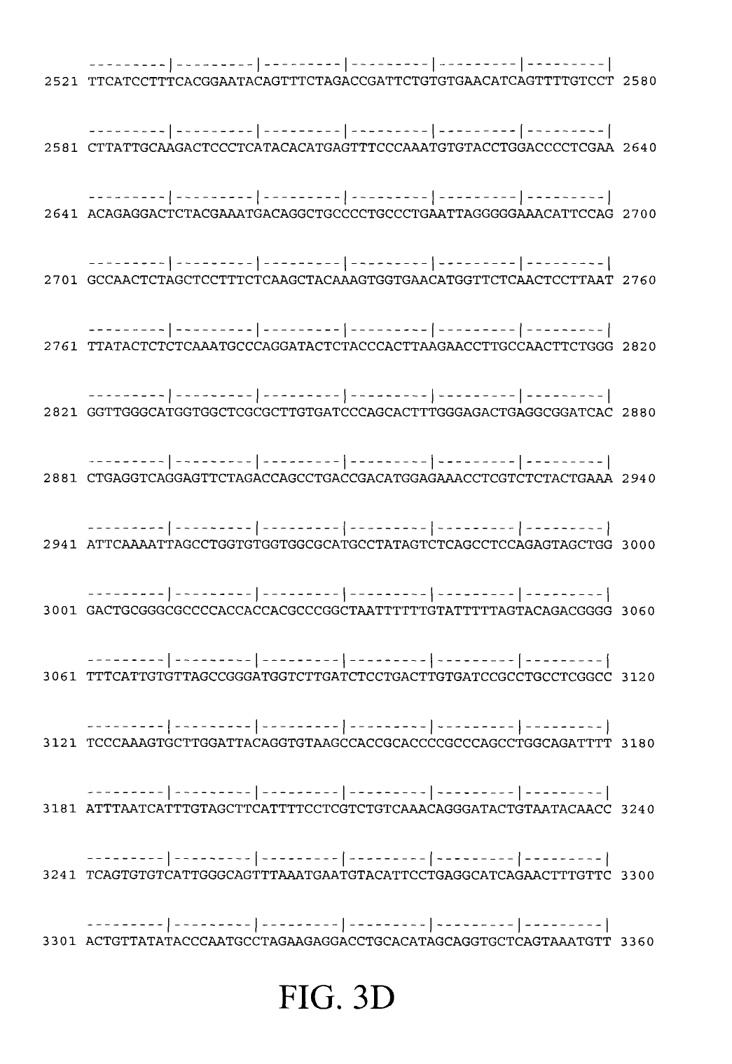

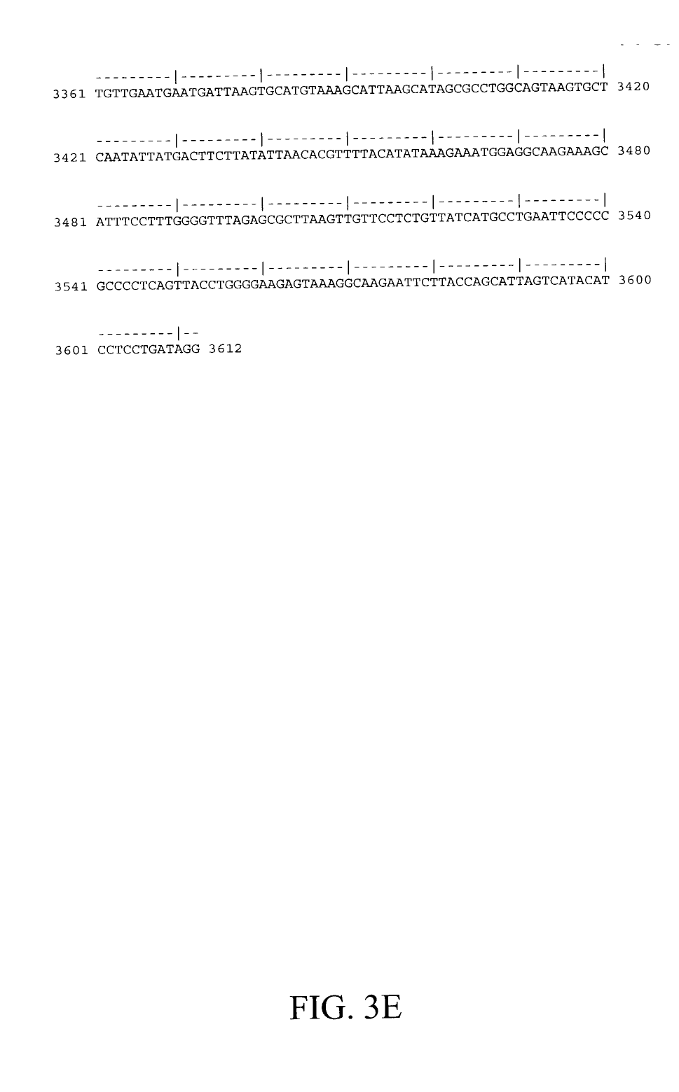

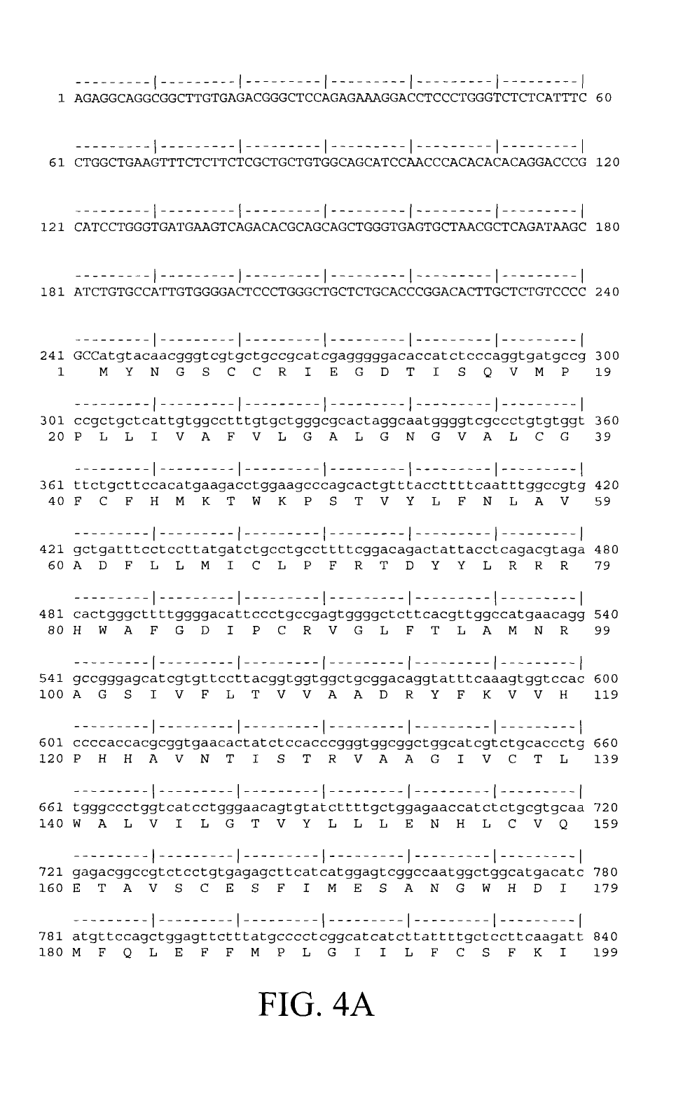

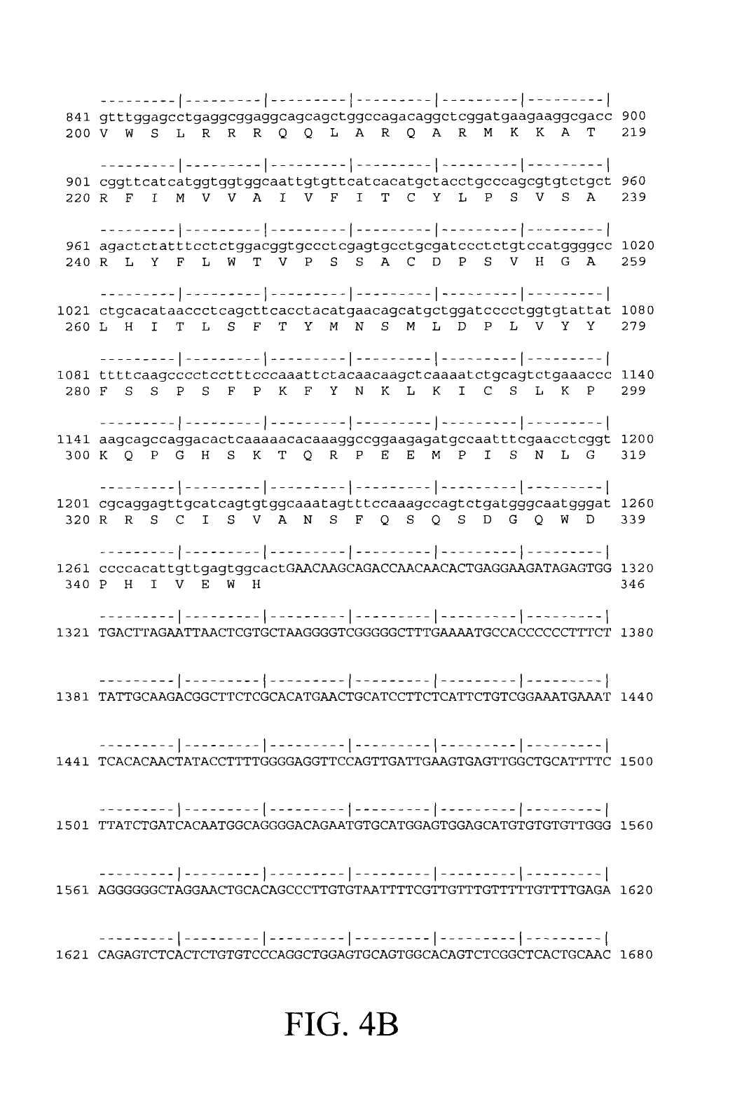

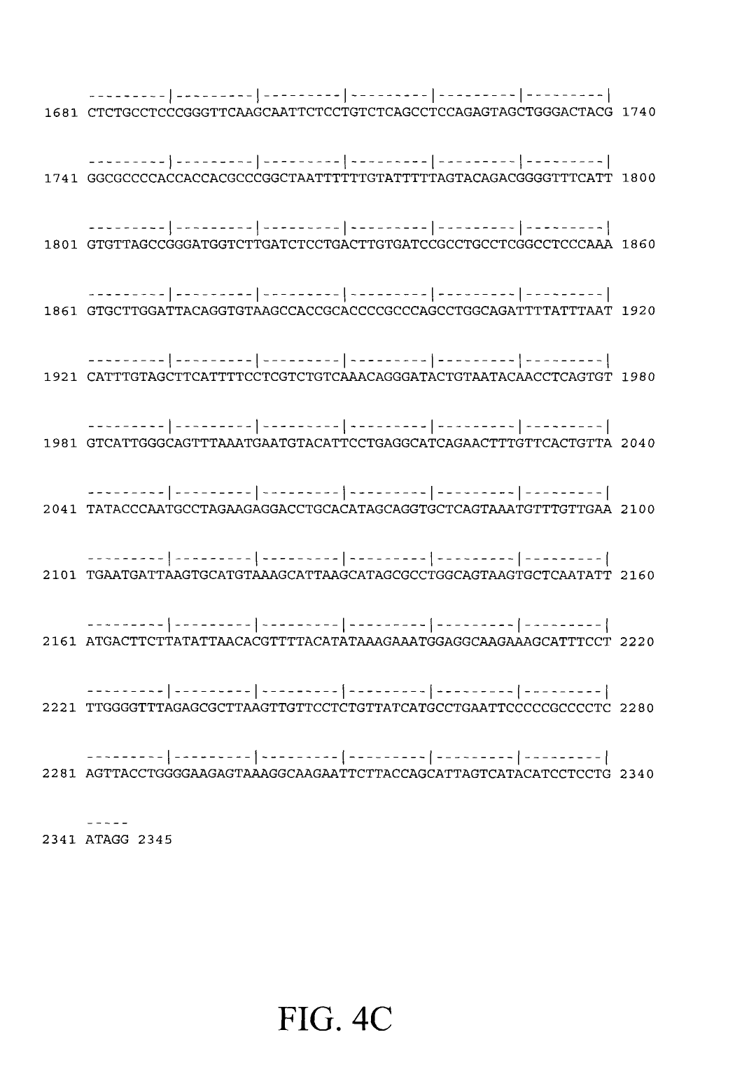

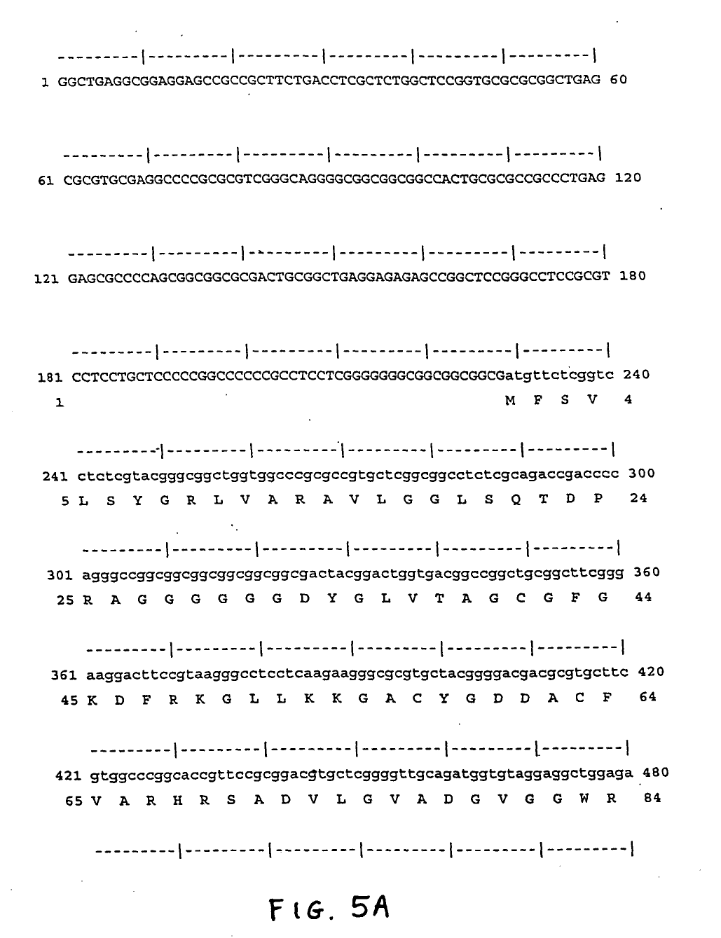

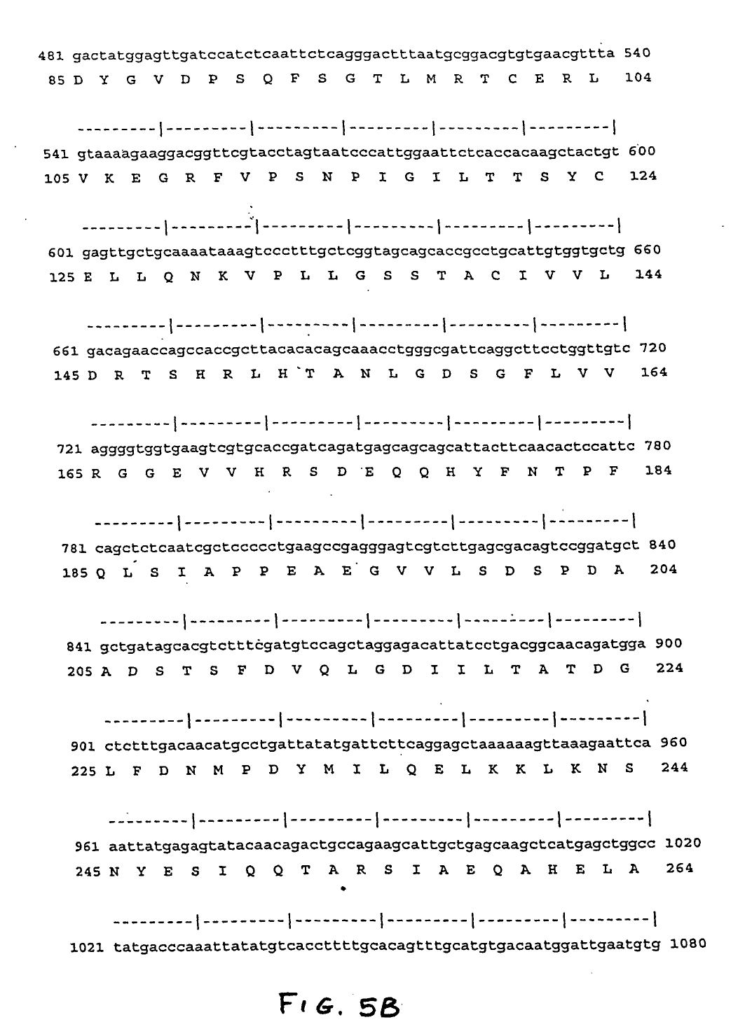

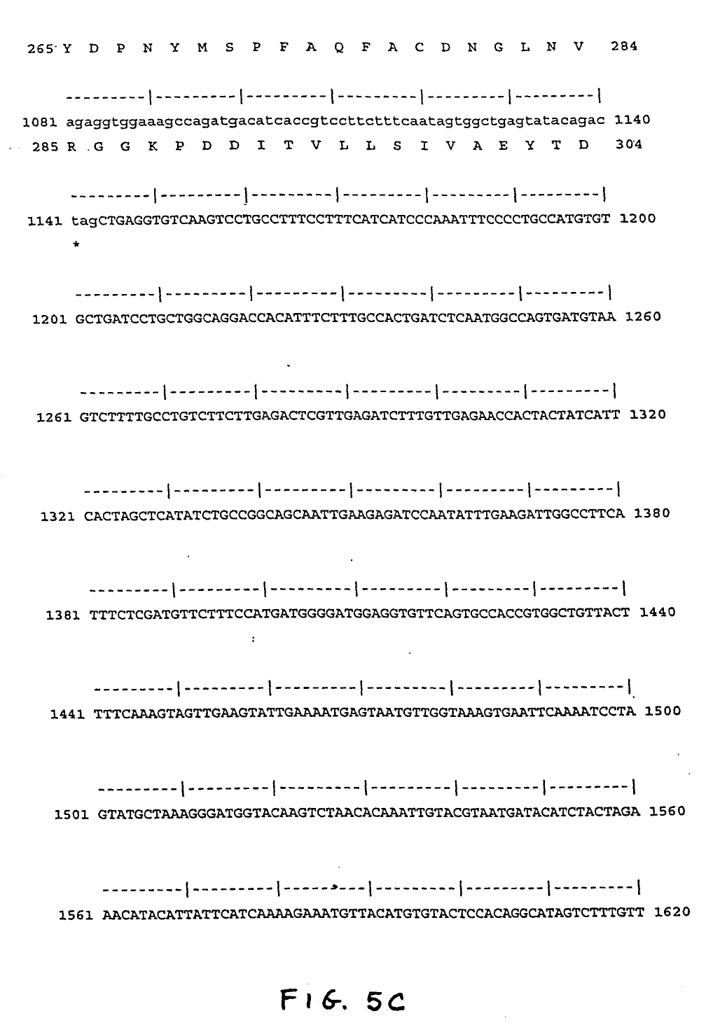

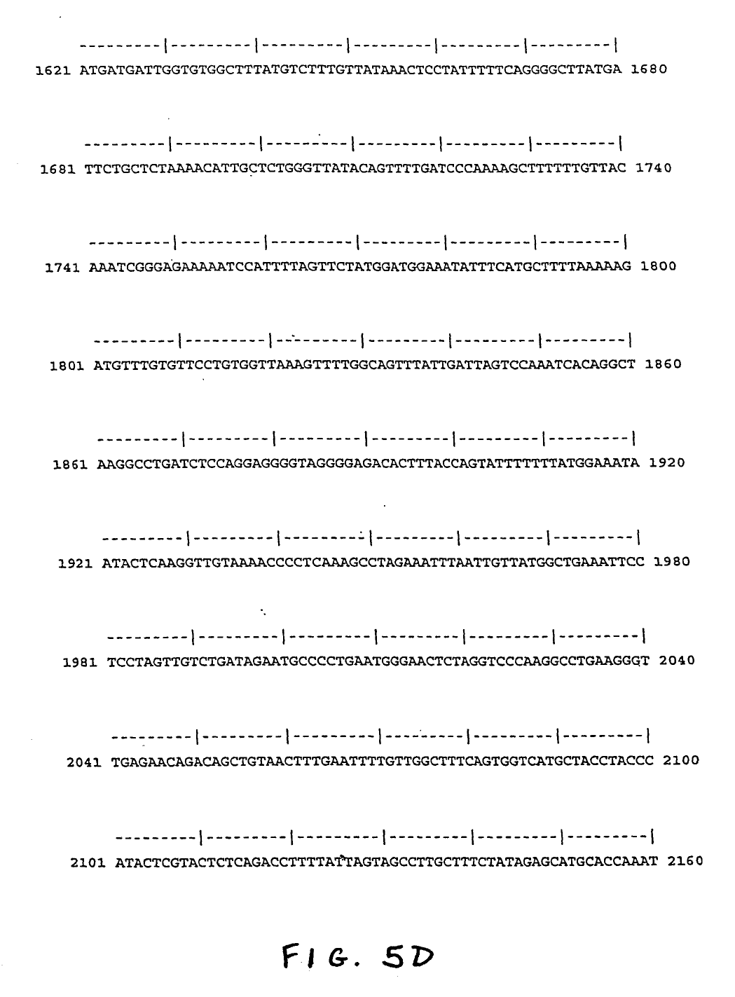

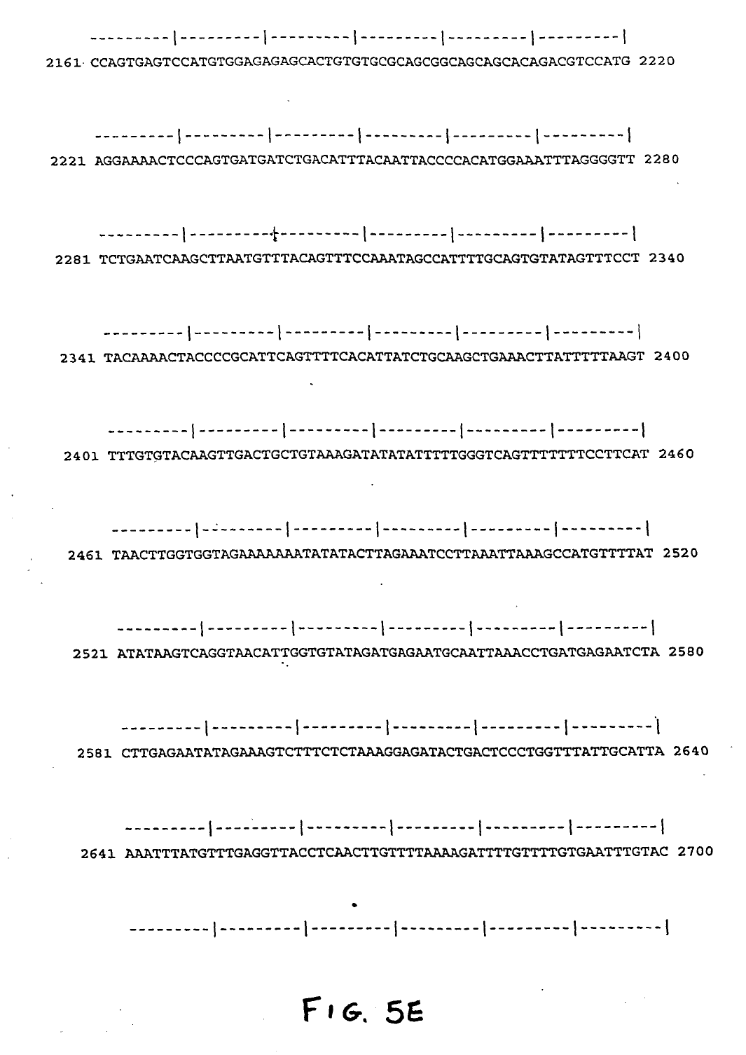

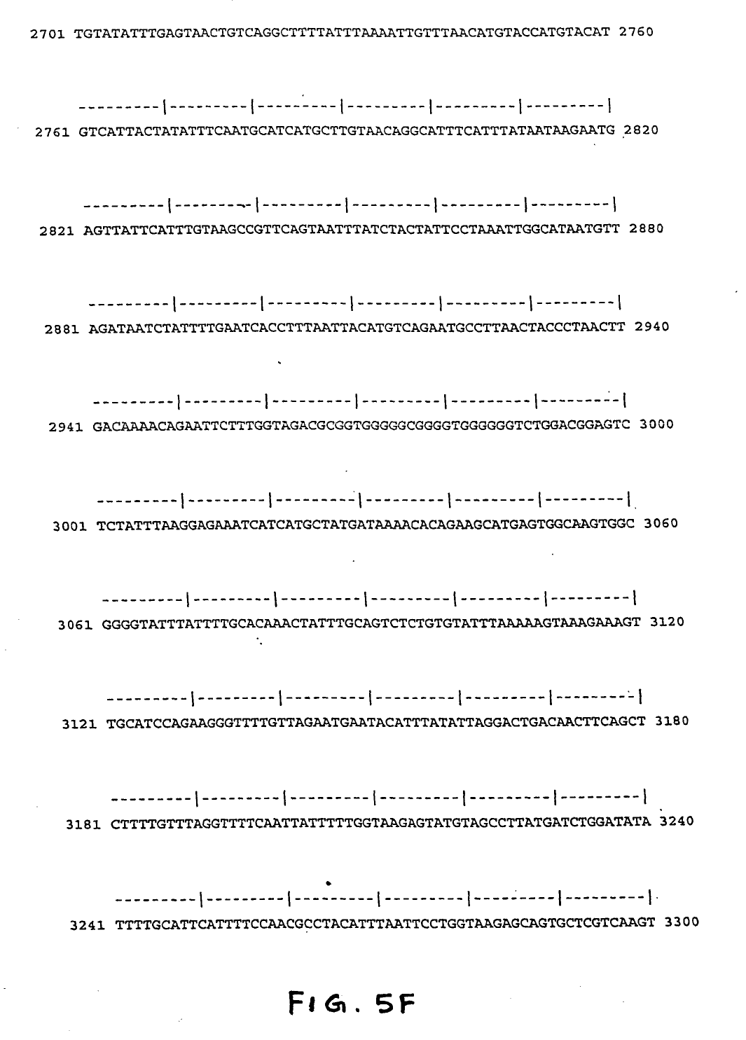

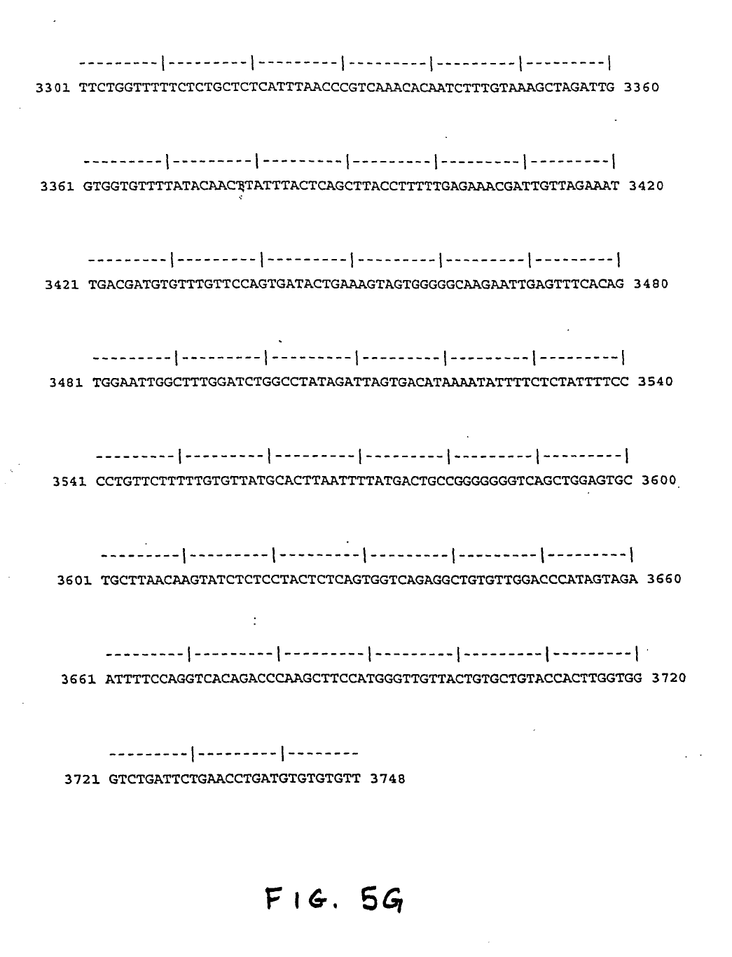

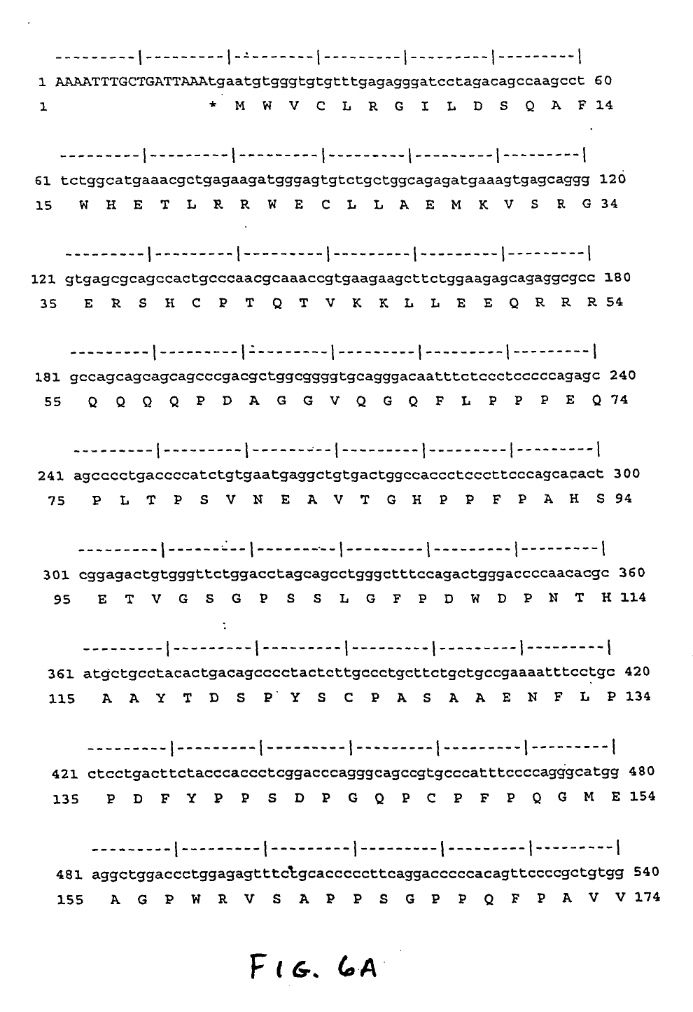

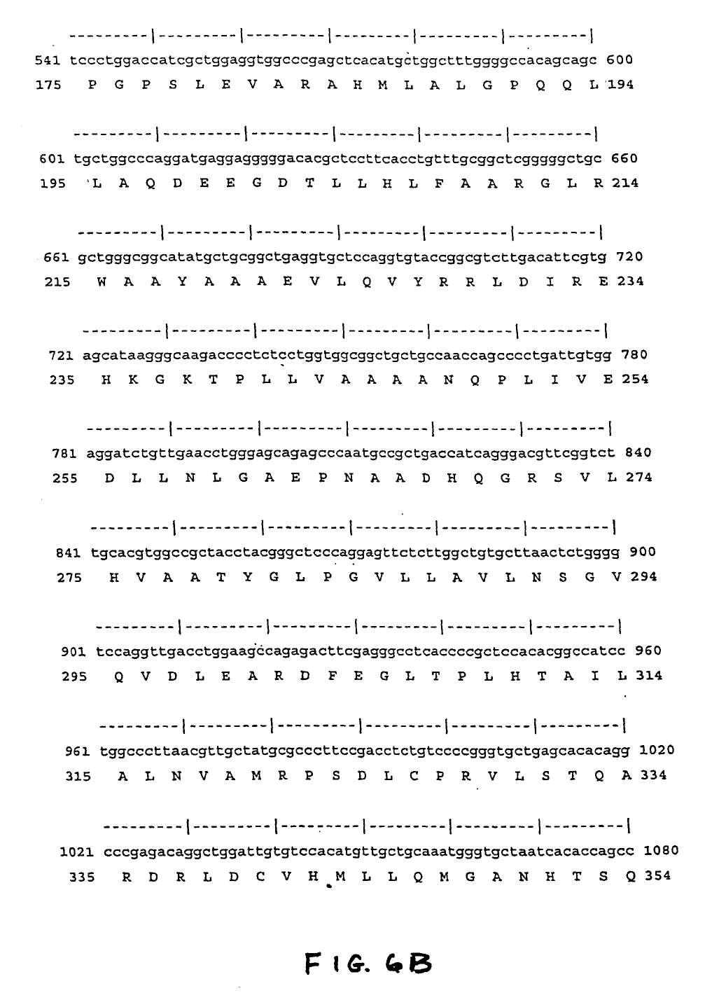

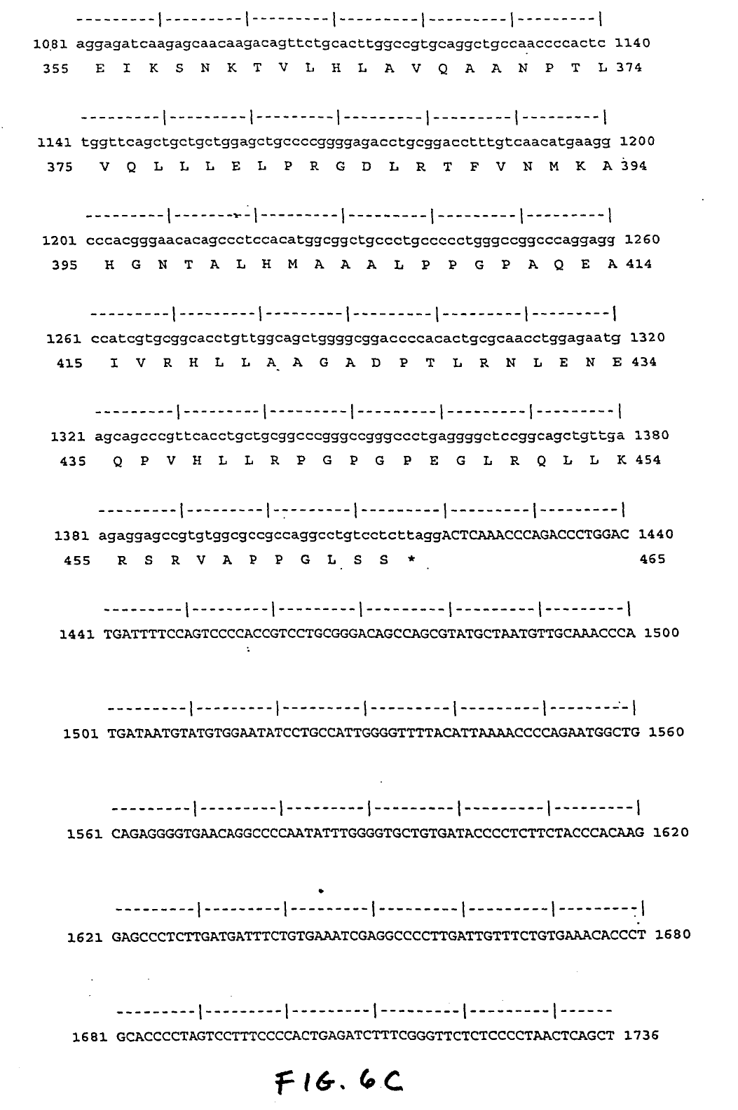

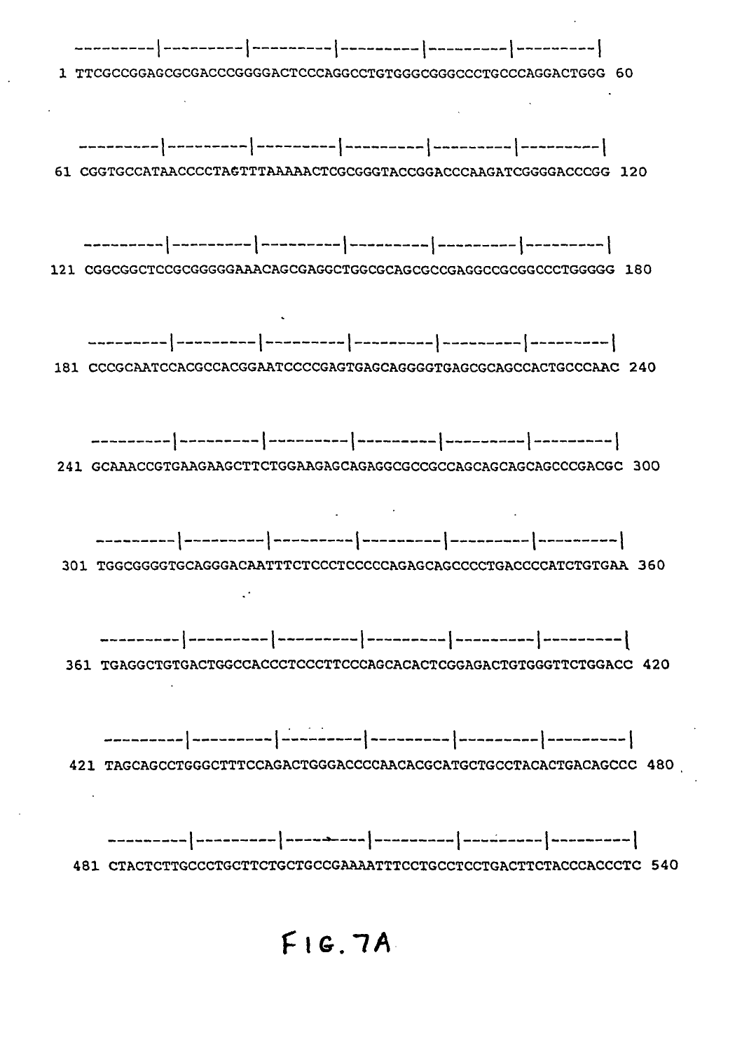

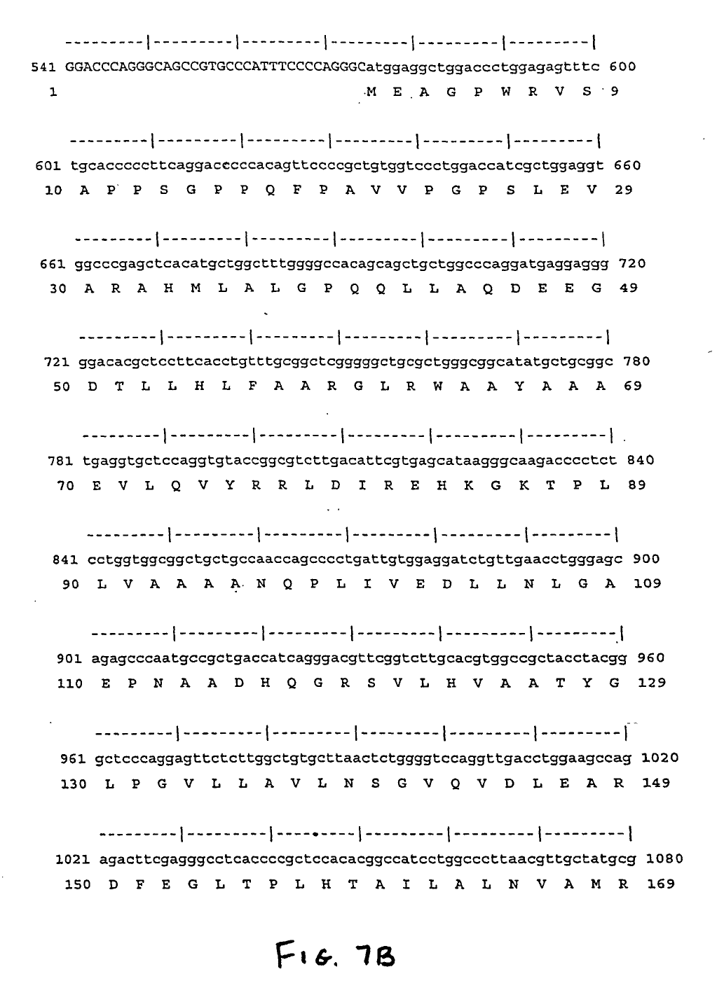

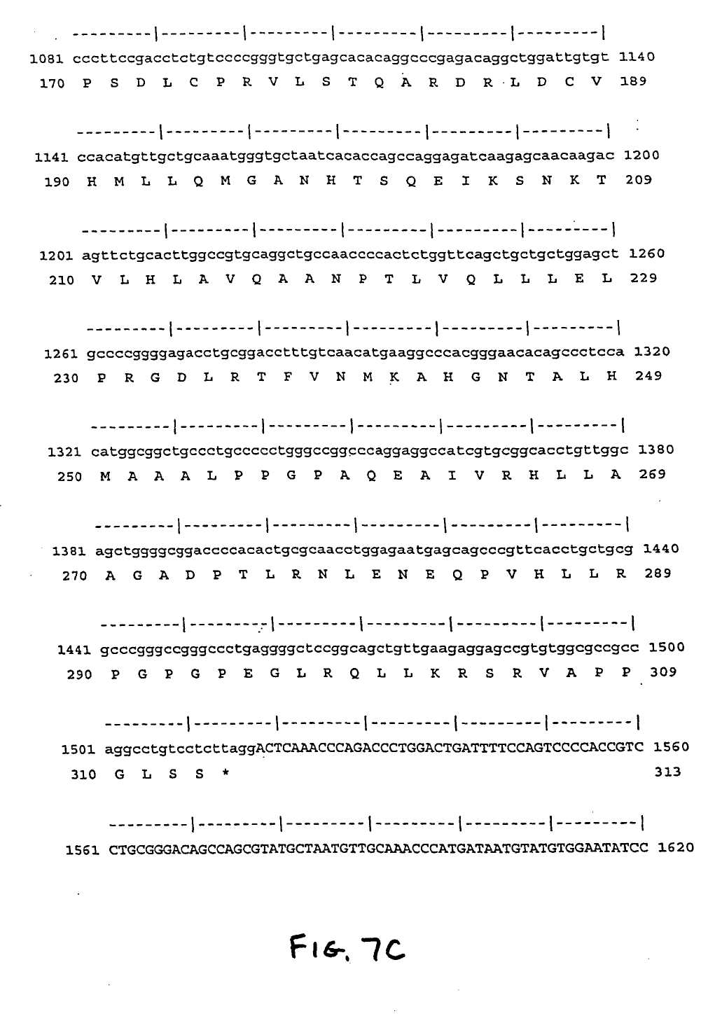

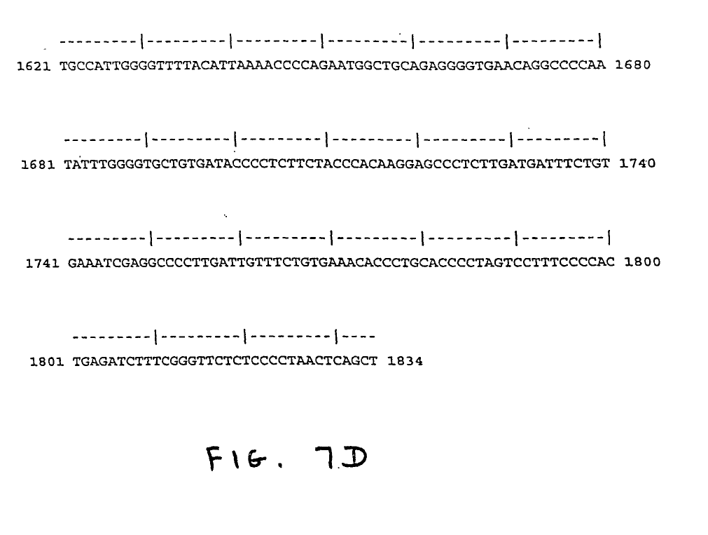

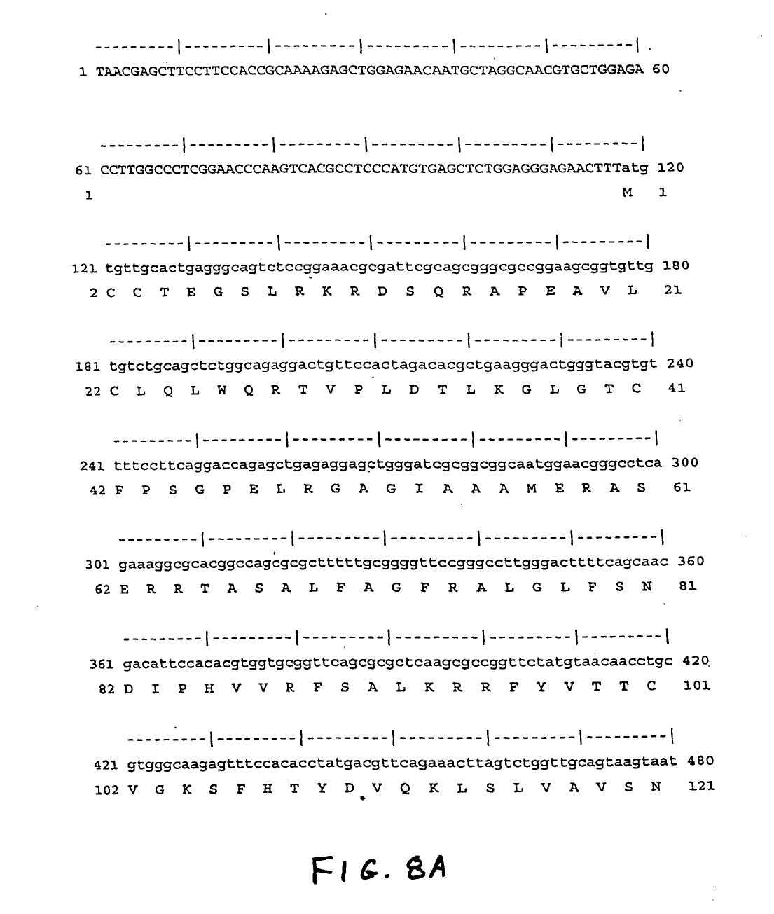

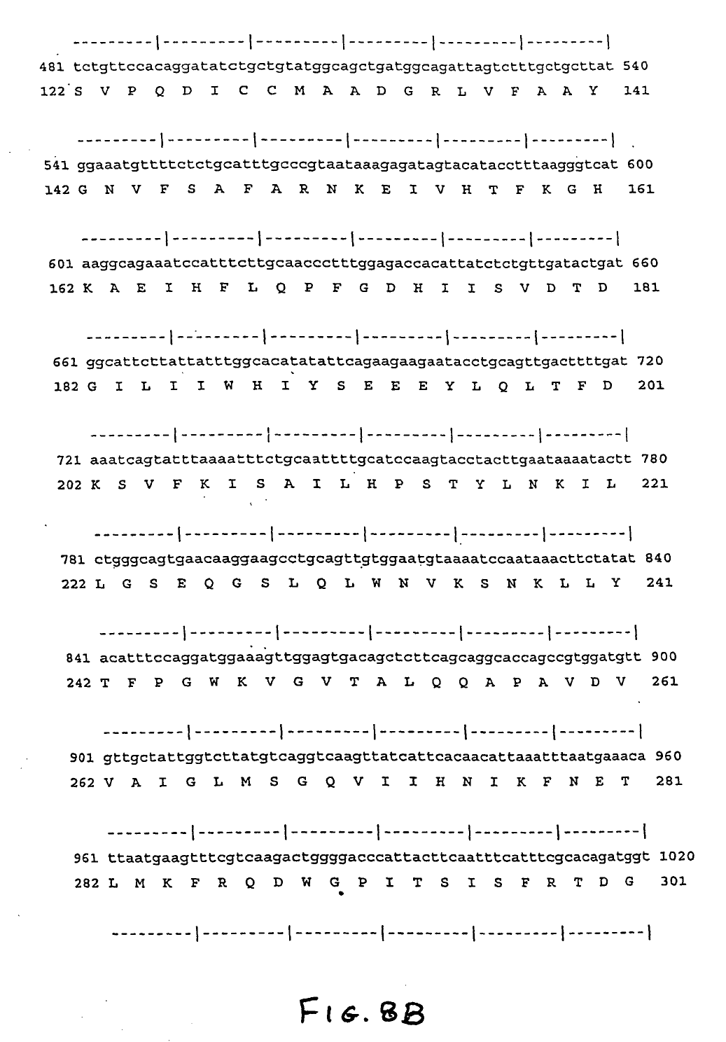

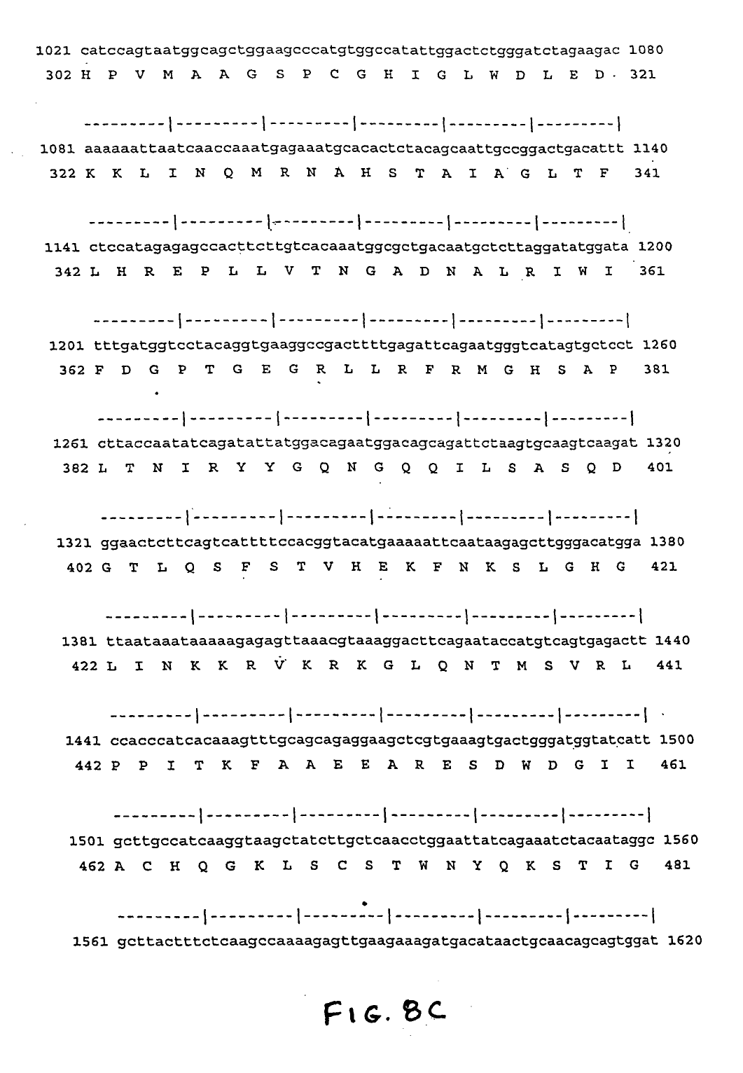

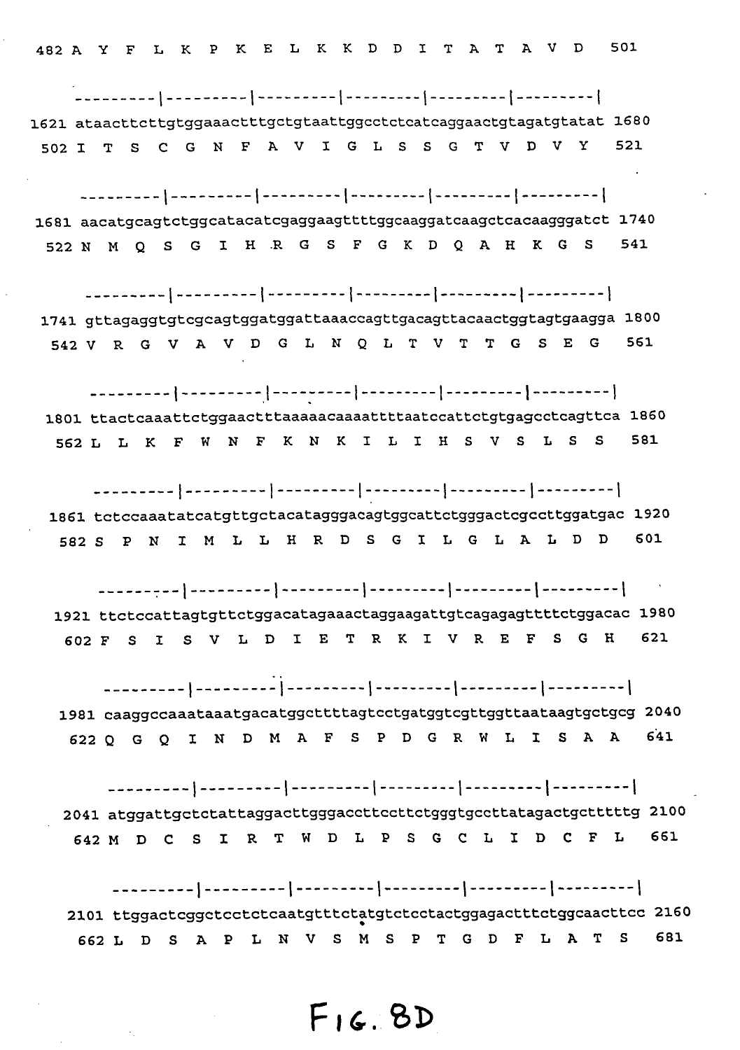

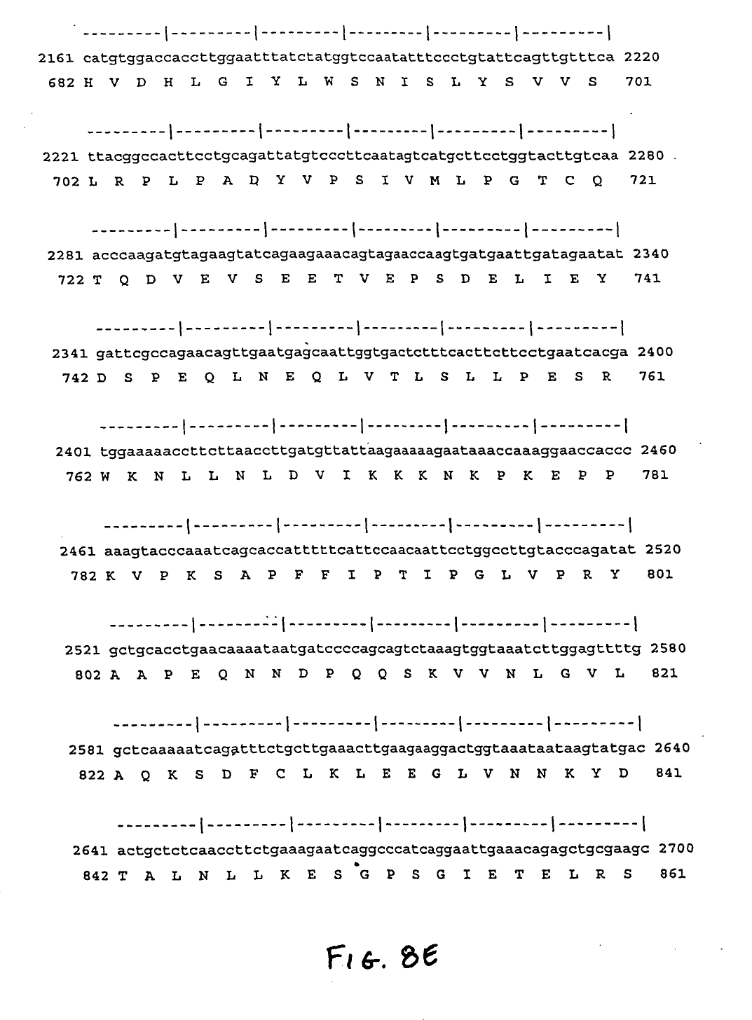

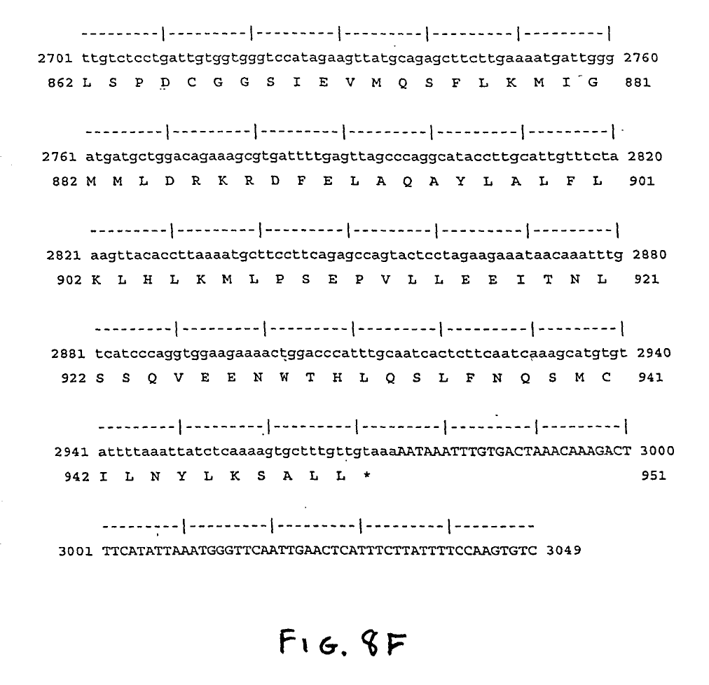

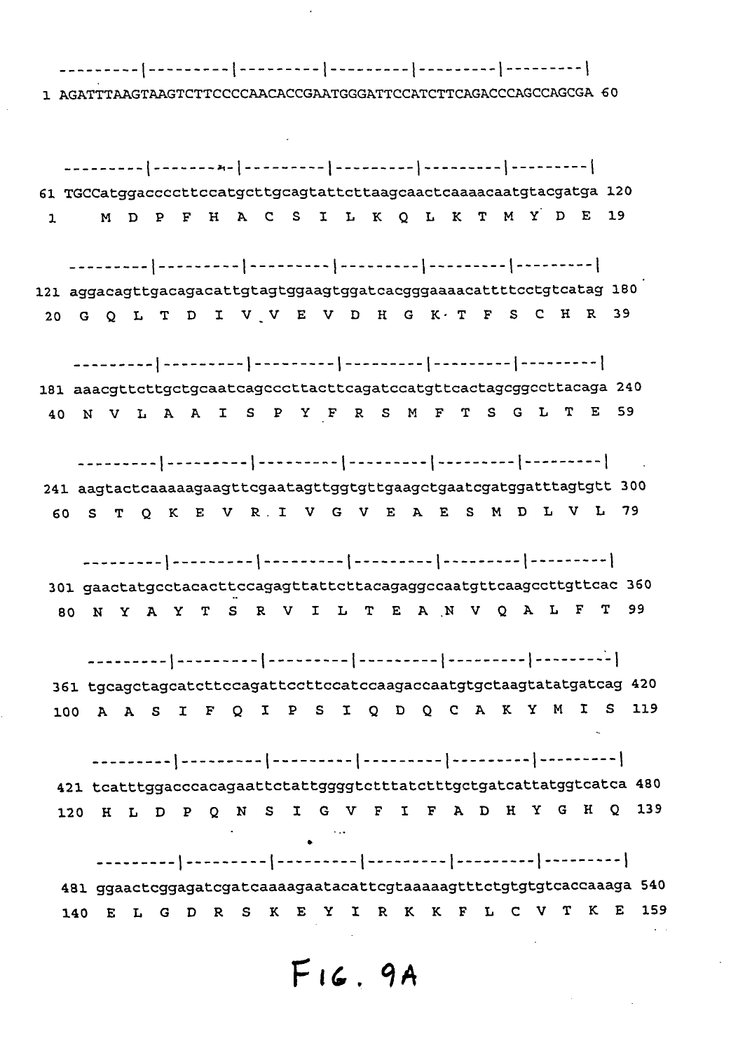

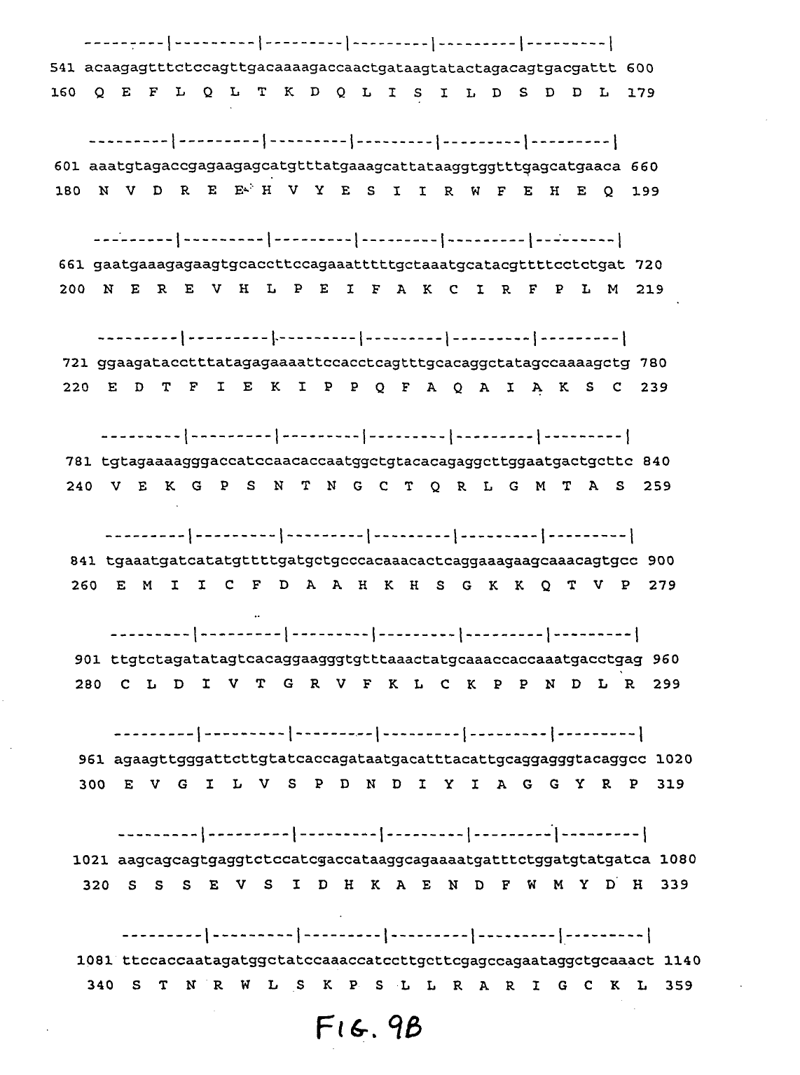

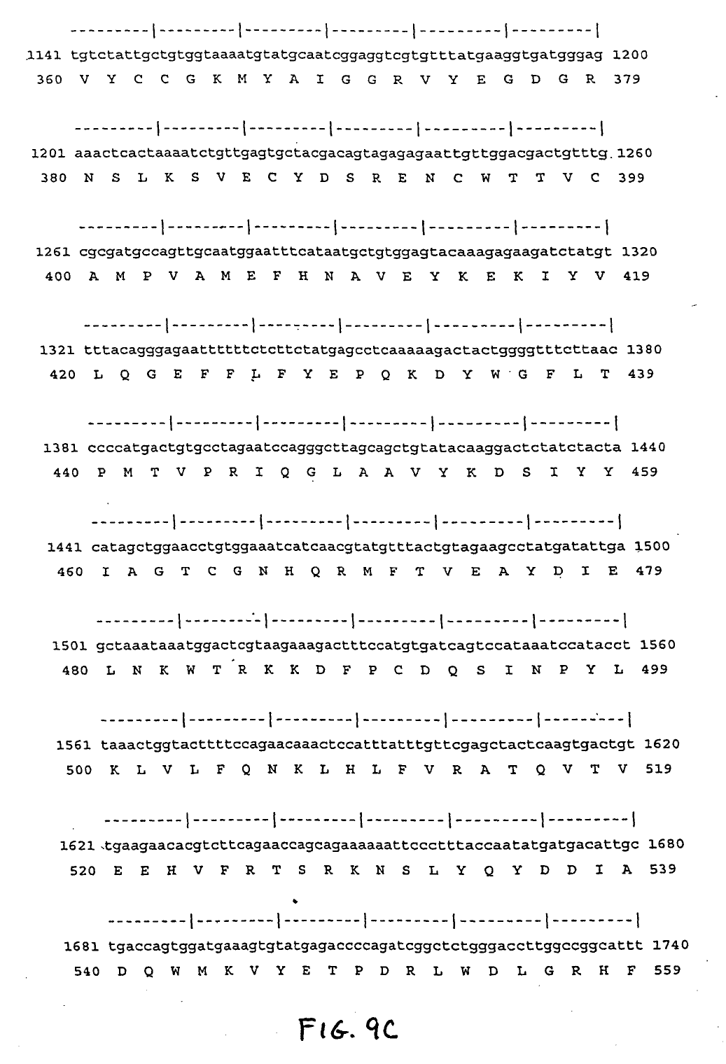

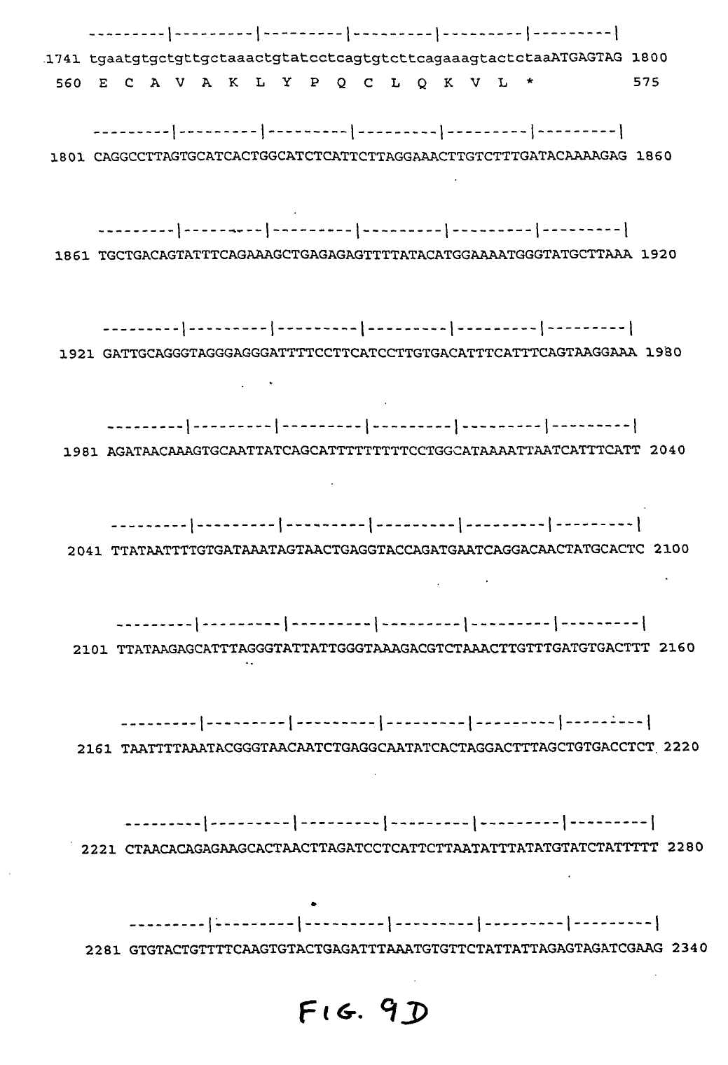

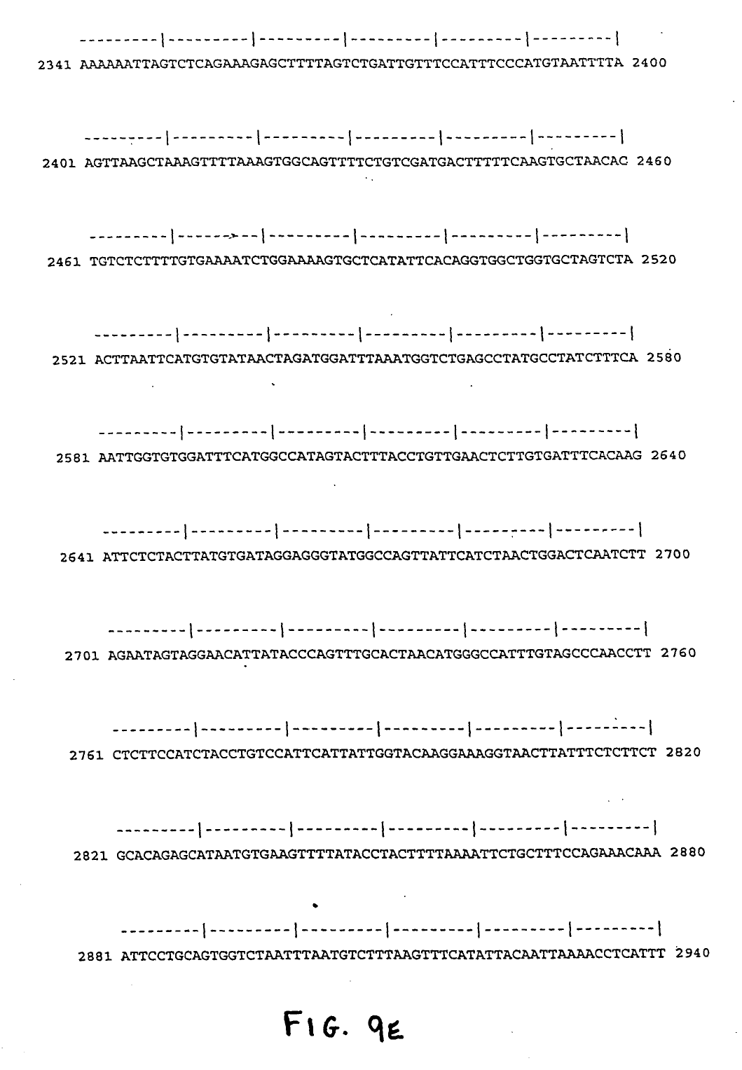

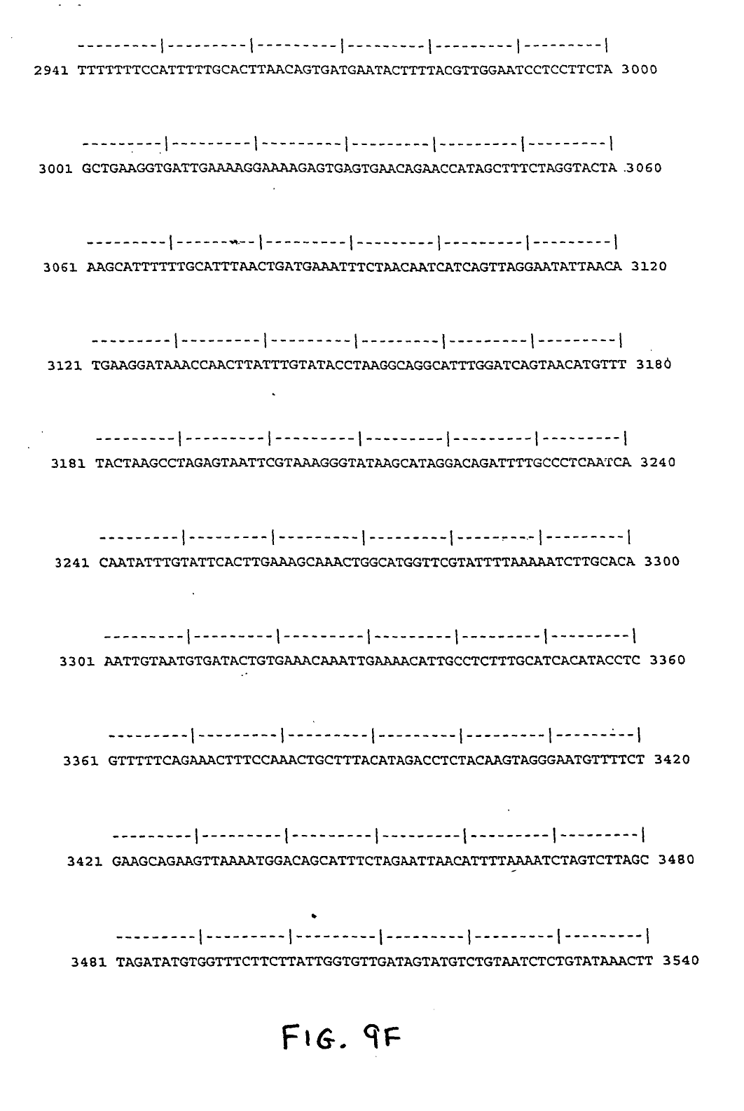

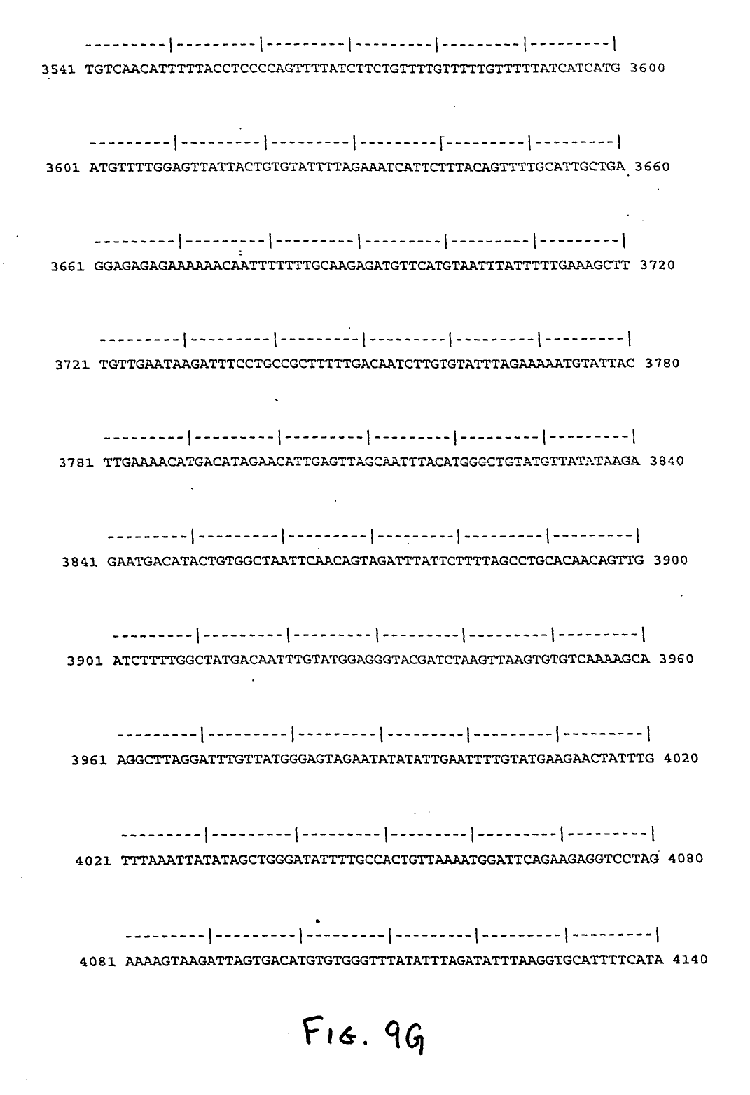

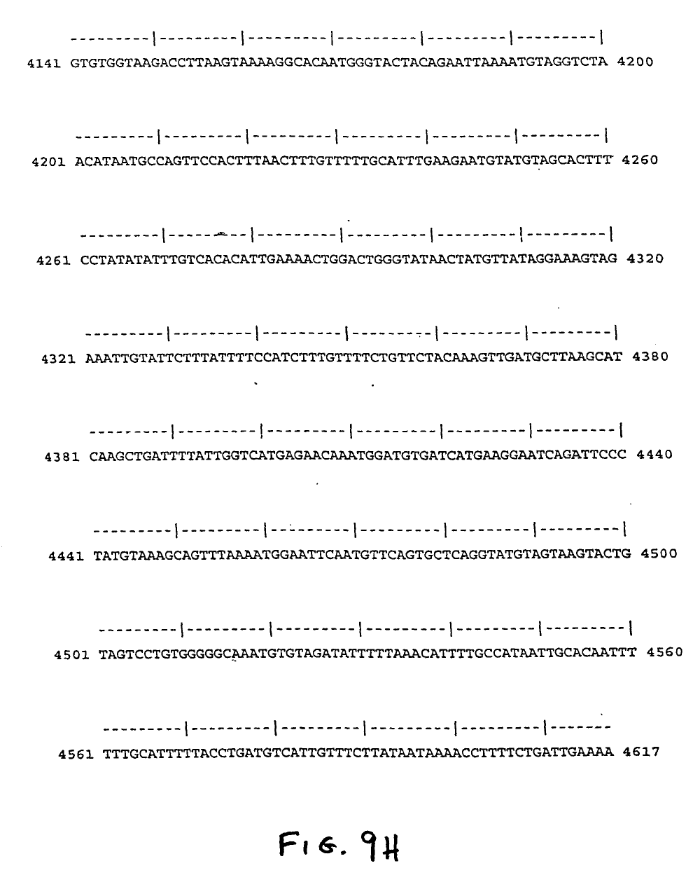

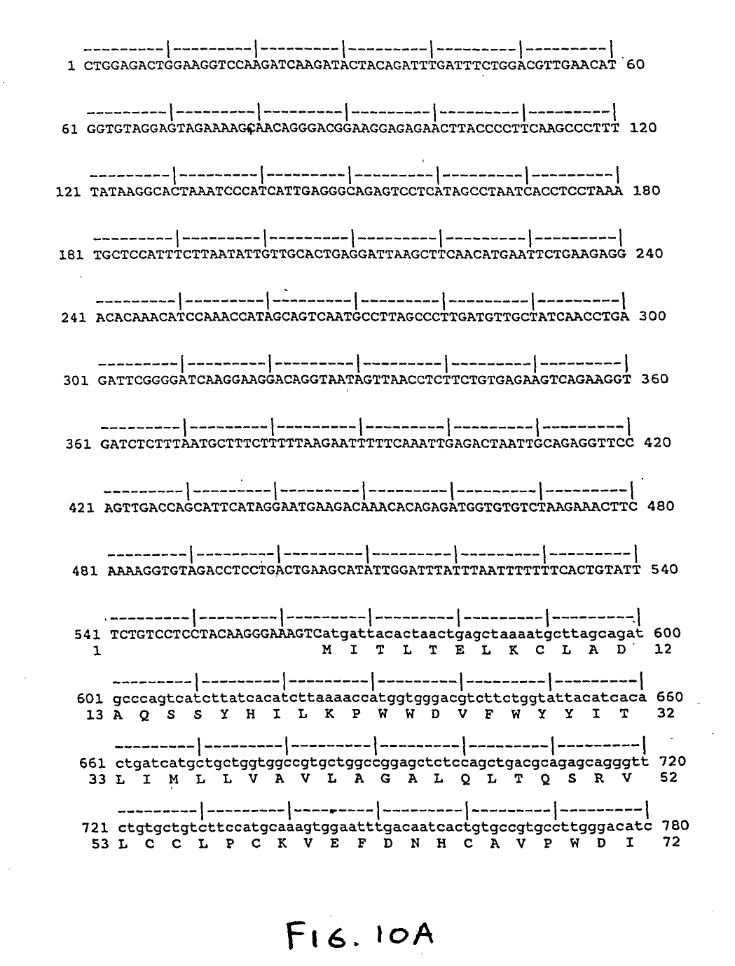

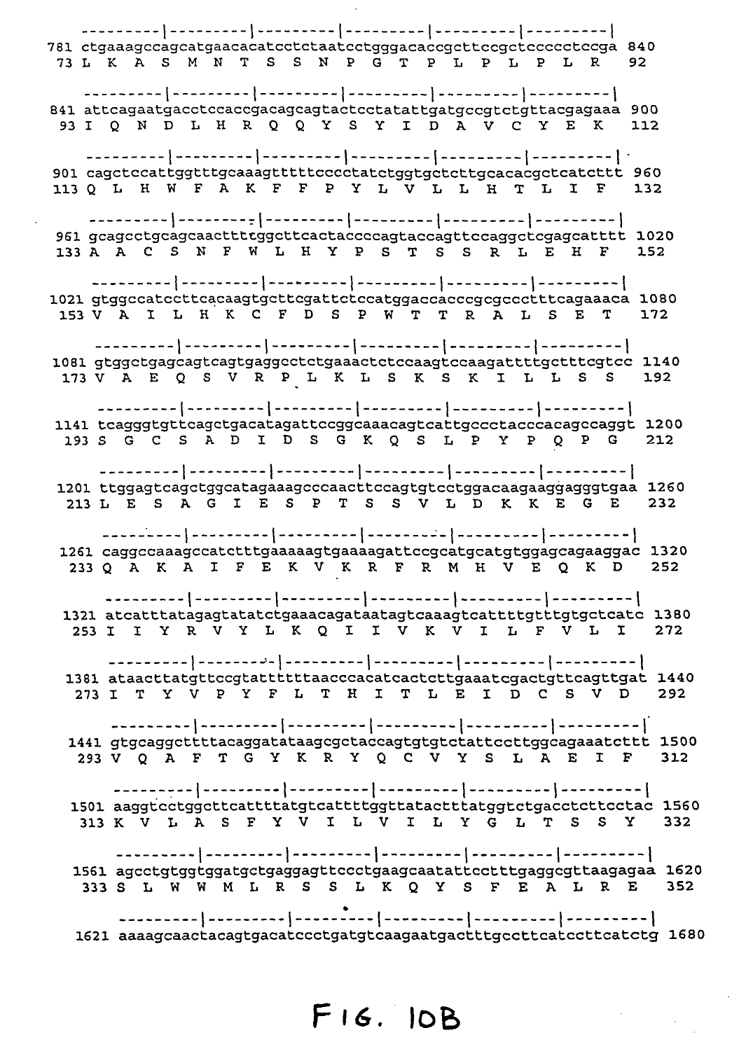

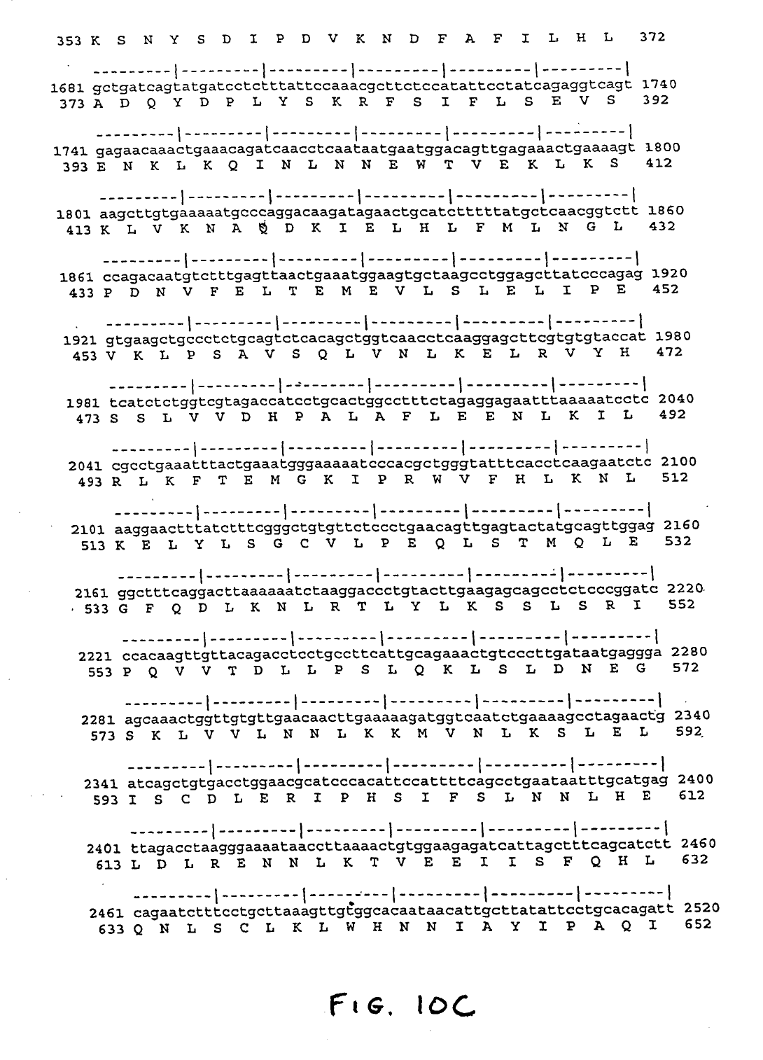

In a specific embodiment of the present invention, such TCAPs, whether produced by recombinant DNA techniques or by chemical synthetic methods or by purification of native proteins, include but are not limited to those containing, as a primary amino acid sequence, all or part of the amino acid sequence substantially as depicted in FIGS. 1A-1E (SEQ ID NOS: 3), FIGS. 2A-2D (SEQ ID NO: 4), FIGS. 3A-3D and 4A-4C (SEQ ID NO: 7), FIGS. 5A-5G (SEQ ID NO: 9), FIGS. 6A-6C (SEQ ID NOS: 11), FIGS. 7A-7D (SEQ ID NO: 13), FIGS. 8A-8F (SEQ ID NO: 15), FIGS. 9A-9H (SEQ ID NO: 17) and FIGS. 10A-10E (SEQ ID NO: 19), as well as fragments and other derivatives thereof, including proteins homologous thereto.

### 5.4. STRUCTURE OF TCAP-ENCODING GENES AND ENCODED PROTEINS

The structure of the genes encoding TCAPs, and the encoded TCAPs, can be analyzed by various methods known in the art, as described in the following sections.

### 5.4.1. GENETIC ANALYSIS

The cloned DNA or cDNA corresponding to a TCAP-encoding gene can be analyzed by methods including, but not limited to, Southern hybridization (Southern, E. M., J. Mol. Biol. 98:503-517 (1975)), northern hybridization (see e.g., Freeman et al., Proc. Natl. Acad. Sci. U.S.A. 80:4094-4098 (1983)), restriction endonuclease mapping (Maniatis, T., Molecular Cloning, A Laboratory, Cold Spring Harbor, N.Y. (1982)), and DNA sequence analysis. Polymerase chain reaction (PCR; U.S. Pat. Nos. 4,683,202, 4,683,195 and 4,889,818; Gyllenstein et al., Proc. Natl. Acad. Sci. U.S.A. 85:7652-7656 (1988); Ochman et al., Genetics 120:621-623 (1988); Loh et al., Science 243:217-220 (1989)) followed by Southern hybridization with a probe specific to one of the TCAP-encoding genes can allow the detection of that particular TCAP-encoding gene in DNA from various cell types from various vertebrate sources. Methods of amplification other than PCR are commonly known and can also be employed. In one embodiment, Southern hybridization can be used to determine the genetic linkage of a particular TCAP gene. Northern hybridization analysis can be used to determine the expression of a particular TCAP gene. Various cell types, at various states of development or activity can be tested for expression of a particular TCAP gene. In one preferred embodiment, screening arrays comprising probes homologous to the exons of particular TCAP-encoding genes are used to determine the state of expression of these genes, or specific exons of these genes, in various cell types, under particular environmental or perturbance conditions, or in various vertebrates. The stringency of the hybridization conditions for both Southern and northern hybridization can be manipulated to ensure detection of nucleic acids with the desired degree of relatedness to the specific probe used. Modifications of these methods and other methods commonly known in the art can be used.

Restriction endonuclease mapping can be used to roughly determine the genetic structure of a TCAP gene. Restriction maps derived by restriction endonuclease cleavage can be confirmed by DNA sequence analysis. The genetic structure of a TCAP gene can also be determined using scanning oligonucleotide arrays, wherein the expression of one exon is correlated with the expression of a plurality of neighboring exons, such that the correlation indicates the correlated exons are contained within the same gene. The structure so determined can be confirmed by PCR.

DNA sequence analysis can be performed by any techniques known in the art, including but not limited to the method of Maxam and Gilbert, Meth. Enzymol. 65:499-5601 (1980), the Sanger dideoxy method (Sanger, F., et al., Proc. Natl. Acad. Sci. U.S.A. 74:5463 (1977)), the use of T7 DNA polymerase (Tabor and Richardson, U.S. Pat. No. 4,795,699), or use of an automated DNA Sequenator (e.g., Applied Biosystems, Foster City, Calif.). The sequencing method may use radioactive or fluorescent labels.

### 5.4.2. PROTEIN ANALYSIS

The amino acid sequence of a particular TCAP can be derived by deduction from the DNA sequence, or alternatively, by direct sequencing of the protein, e.g., with an automated amino acid sequencer.

The protein sequence of a TCAP can be characterized by a hydrophilicity analysis (Hopp and Woods, Proc. Natl. Acad. Sci. U.S.A. 78:3824 (1981)). A hydrophilicity profile is used to identify the hydrophobic and hydrophilic regions of a TCAP and the corresponding regions of the gene sequence which encode such regions.

Secondary structural analysis (Chou and Fasman, Biochemistry 13:222 (1974)) can also be done, to identify regions of particular TCAPs that assume specific secondary structures, such as α-helices, β-pleated sheets or turns.

Manipulation, translation, secondary structure prediction, open reading frame prediction and plotting, as well as determination of sequence homologies, can also be accomplished using computer software programs and nucleotide and protein sequence databases available in the art. Protein and/or nucleotide sequence homologies to known proteins or DNA sequences can be used to deduce the likely function of a particular TCAP, or domains thereof.

Other methods of structural analysis can also be employed. These include but are not limited to X-ray crystallography (Engstom, Biochem. Exp. Biol. 11:7-13 (1974)) and computer modeling (Fletterick, and Zoller, (eds.), Computer Graphics and Molecular Modeling, in Current Communications in Molecular Biology, Cold Spring Harbor Laboratory, Cold Spring Harbor, N.Y. (1986)).

In addition to determinations of the TCAP protein structure, the invention provides method of identifying a molecule that specifically binds to a ligand selected from the group consisting of a TCAP, a fragment of a TCAP comprising a domain of the TCAP, and a nucleic acid encoding the TCAP or fragment thereof, comprising (a) contacting said ligand with a plurality of molecules under conditions conducive to binding between said ligand and the molecules; and (b) identifying a molecule within said plurality that specifically binds to said ligand.

### 5.5. GENERATION OF ANTIBODIES TO TCAPS AND DERIVATIVES THEREOF

According to the invention, a TCAP, its fragments, or other derivatives thereof may be used as an immunogen to generate antibodies which immunospecifically bind such an immunogen. Such antibodies include but are not limited to polyclonal, monoclonal, chimeric and single chain antibodies, as well as Fab fragments and an Fab expression library. In a specific embodiment, antibodies to human TA-GPCR, TA-GAP, TA-NFKBH, TA-KRP, TA-PP2C, TA-WDRP or TA-LRRP are produced. In another embodiment, antibodies to a domain of a particular TCAP are produced. In a specific embodiment, fragments of a TCAP protein identified as hydrophilic are used as immunogens for antibody production.

Various procedures known in the art may be used for the production of polyclonal antibodies to a specific TCAP, or derivative thereof In a particular embodiment, rabbit polyclonal antibodies to an epitope of a TCAP encoded by a sequence of SEQ ID NO: 1, SEQ ID NO: 2, SEQ ID NO: 5, SEQ ID NO:6, SEQ ID NO: 8, SEQ ID NO: 10, SEQ ID NO: 12, SEQ ID NO: 14, SEQ ID NO: 16 or SEQ ID NO: 18 or a subsequence thereof, can be obtained. For the production of antibody, various host animals can be immunized by injection with native TCAP, or a synthetic version or derivative (e.g., fragment) thereof, including, but not limited to, rabbits, mice, rats, goats, bovines or horses. Various adjuvants may be used to increase the immunological response, depending on the host species. Adjuvants that may be used according to the present invention include, but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

For preparation of monoclonal antibodies directed toward a TCAP sequence or derivative thereof, any technique which provides for the production of antibody molecules by continuous cell lines in culture may be used. For example, monoclonal antibodies may be prepared by the hybridoma technique originally developed by Kohler and Milstein, Nature 256:495-497 (1975), as well as the trioma technique, the human B-cell hybridoma technique (Kozbor et al., Immunol. Today 4:72 (1983)), or the EBV-hybridoma technique (Cole et al., in MONOCLONAL ANTIBODIES AND CANCER THERAPY, Alan R. Liss, Inc., pp. 77-96 (1985)). In an additional embodiment of the invention, monoclonal antibodies can be produced in germ-free animals utilizing recent technology (PCT/US90/02545). According to the invention, human antibodies may be used and can be obtained by using human hybridomas (Cote et al., Proc. Natl. Acad. Sci. U.S.A., 80:2026-2030 (1983)) or by transforming human B cells with EBV virus in vitro (Cole et al., in MONOCLONAL ANTIBODIES AND CANCER THERAPY, Alan R. Liss, pp. 77-96 (1985)). Furthermore, according to the invention, techniques developed for the production of “chimeric antibodies” (Morrison et al., Proc. Natl. Acad. Sci. U.S.A. 81:6851-6855 (1984); Neuberger et al., Nature 312:604-608 (1984); Takeda et al., Nature 314:452-454 (1985)) can be used, wherein genes from a mouse antibody molecule specific to a particular TCAP are spliced to genes encoding a human antibody molecule of appropriate biological activity can be used; such antibodies are within the scope of this invention.

According to the invention, techniques described for the production of single chain antibodies (U.S. Pat. No. 4,946,778) can be adapted to produce single chain antibodies specific to a particular TCAP. An additional embodiment of the invention utilizes the techniques described for the construction of Fab expression libraries (Huse et al., Science 246:1275-1281 (1988)) to allow rapid and easy identification of monoclonal Fab fragments with the desired specificity for particular TCAPs or derivatives thereof. Antibody fragments which contain the idiotype of the molecule can be generated by known techniques. For example, such fragments include but are not limited to: the F(ab′), fragment which can be produced by pepsin digestion of the antibody molecule; the Fab′ fragments which can be generated by reducing the disulfide bridges of the F(ab′), fragment, the Fab fragments which can be generated by treating the antibody molecule with papain and a reducing agent, and Fv fragments.

In the production of antibodies, screening for the desired antibody can be accomplished by techniques known in the art, e.g. ELISA (enzyme-linked immunosorbent assay), RIA (radioimmunoassay) or RIBA (recombinant immunoblot assay). For example, to select antibodies which recognize a specific domain of a TCAP, one may assay generated hybridomas for a product which binds to a TCAP fragment containing such domain. For selection of an antibody that specifically binds a first TCAP homolog but which does not specifically bind a second, different TCAP homolog, one can select on the basis of positive binding to the first TCAP homolog and a lack of binding to the second TCAP homolog.

Antibodies specific to a domain of a TCAP are also provided. The foregoing antibodies can be used in methods known in the art relating to the localization and activity of the TCAP sequences of the invention, e.g., for imaging these proteins, measuring levels thereof in appropriate physiological samples, in diagnostic methods, etc.

In another embodiment of the invention, antibodies to particular TCAPs, and antibody fragments thereof containing the binding domain are therapeutics (see infra). In a preferred embodiment, the antibodies are isolated or purified.

### 5.6. TCAPS AND TCAP DERIVATIVES

The invention further relates to specific TCAPs and derivatives (including but not limited to fragments) of these specific TCAPs (e.g.,TA-GPCR, TA-GAP, TA-NFKBH, TA-KRP, TA-PP2C, TA-WDRP or TA-LRRP). Nucleic acids encoding derivatives and protein of these TCAPs are also provided. In one embodiment, specific TCAPs are encoded by the associated TCAP nucleic acids described in Section 5.1 supra.

The production and use of derivatives produced through modification of TCAP-encoding genes are within the scope of the present invention. In a specific embodiment, the derivative is functionally active, i.e., capable of exhibiting one or more functional activities associated with a full-length, wild-type TCAP. As one example, such derivatives that have the desired immunogenicity or antigenicity can be used, for example, in immunoassays, for immunization, for inhibition of the activity of a specific TCAP, etc. As another example, such derivatives that substantially have the desired TCAP activity, or which are phosphorylated or dephosphorylated, are provided. Derivatives that retain, or alternatively lack or inhibit, a desired TCAP property of interest, a specific activity, such as activity associated with G-coupled proteins (TA-GPCR), GTPase-inducing activity (TA-GAP), transcriptional activation activity (TA-NFKBH), protease activity (TA-PP2C) or G-protein activity (TA-WDRP); inhibition of these activities), can be used as inducers, or inhibitors, respectively, of such a property and its physiological correlates. A specific embodiment relates to a TA-GPCR, TA-GAP, TA-NFKBH, TA-KRP, TA-PP2C, TA-WDRP of TA-LRRP fragment that can be bound by an antibody directed to the corresponding native TCAP. Derivatives of particular TCAPs can be tested for the desired activity by procedures known in the art, including but not limited to the assays described in Section 5.7.

In particular, derivatives of TCAPs can be made by altering the nucleotide sequences encoding them by substitutions, additions or deletions that provide for functionally equivalent protein molecules. In a specific embodiment, the alteration is made in a nucleic acid sequence encoding all or part of TA-GPCR, TA-GAP, TA-NFKBH, TA-KRP, TA-PP2C, TA-WDRP or TA-LRRP. Due to the degeneracy of nucleotide coding sequences, other DNA sequences that encode substantially the same amino acid sequence as a TCAP-encoding gene may be used in the practice of the present invention. These include but are not limited to nucleotide sequences comprising all or portions of TCAP-encoding genes that are altered by the substitution of different codons that encode the same amino acid residue within the sequence, thus producing a silent change.

Likewise, the TCAP derivatives of the invention include, but are not limited to, those containing, as a primary amino acid sequence, all or part of the amino acid sequence of a TA-GPCR, TA-GAP, TA-NFKBH, TA-KRP, TA-PP2C, TA-WDRP or TA-LRRP protein, including altered sequences in which functionally equivalent amino acid residues are substituted for residues within the sequence resulting in a silent or insubstantial change. For example, one or more amino acid residues within the sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

In a specific embodiment of the invention, proteins consisting of or comprising a fragment of a particular TCAP consisting of at least 10 (continuous) amino acids of that TCAP protein is provided. In other embodiments, the fragment consists of at least 20 or 50 amino acids of a particular TCAP. In specific embodiments, such fragments are not larger than 35, 100 or 200 amino acids. Derivatives of TCAPs include but are not limited to those molecules comprising regions that are homologous to TA-GPCR, TA-GAP, TA-NFKBH, TA-KRP, TA-PP2C, TA-WDRP or TA-LRRP or fragments thereof. “Homologous” means that in various embodiments, two amino acid sequences share preferably at least 60% or 70%, more preferably at least 80% or 90%, and even more preferably at least 95% sequence identity over an amino acid sequence of identical size. When the alignment is done by a computer homology program known in the art, such as BLAST (blastp), the percent homology is calculated by dividing the number of amino acids in the TCAP sequence or fragment thereof into the number of amino acids of the TCAP sequence exactly matching the amino acid at the same position in the second sequence, where introduced gaps count as a mismatch, and where conservative changes count as a match. A BLAST comparison can also determine the “sequence similarity” between two proteins, where sequence similarity is defined as a positive score in a BLOSLUM62 scoring matrix comparison of the two sequences.

Derivatives of TCAPs also include molecules whose encoding nucleic acid is capable of hybridizing to a TCAP-encoding sequence, under stringent, moderately stringent, or nonstringent conditions.

The TCAP derivatives of the invention can be produced by various methods known in the art. The manipulations which result in their production can occur at the gene or protein level. For example, the cloned gene sequence of a TCAP gene can be modified by any of numerous strategies known in the art (Maniatis, Molecular Cloning, A Laboratory Manual, 2d. ed., Cold Spring Harbor Laboratory, Cold Spring Harbor, N.Y. (1990)). The sequence can be cleaved at appropriate sites with restriction endonuclease(s), followed by further enzymatic modification if desired, then isolated and ligated in vitro. In the production of a gene encoding a derivative of a TCAP, care should be taken to ensure that the modified gene remains within the same translational reading frame as the TCAP gene, uninterrupted by translational stop signals, in the gene region where the desired TCAP activity is encoded.

Additionally, a TCAP-encoding nucleic acid sequence can be mutated in vitro or in vivo to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, chemical mutagenesis, in vitro site-directed mutagenesis (Hutchinson, et al., J. Biol. Chem. 253:6551(1978)), use of TAB linkers (Pharmacia), PCR using mutagenizing primers, and so forth.

Manipulations of a TCAP sequence may also be made at the protein level. Included within the scope of the invention are TCAP fragments or other derivatives which are differentially modified during or after translation, e.g., by glycosylation, acetylation, phosphorylation, amidation, derivatization by known protecting/blocking groups, proteolytic cleavage, or linkage to an antibody molecule or other cellular ligand. Any of numerous chemical modifications may be carried out by known techniques, including, but not limited to, specific chemical cleavage by cyanogen bromide, trypsin, chymotrypsin, papain, V8 protease, NaBH₄; acetylation, formylation, oxidation, reduction; metabolic synthesis in the presence of tunicamycin; and so forth.

In addition, derivatives of a TCAP can be chemically synthesized. For example, a peptide corresponding to a portion of a TCAP that comprises a desired domain (see Section 5.6.1), or which mediates the desired activity in vitro, can be synthesized by use of a peptide synthesizer. Furthermore, if desired, nonclassical amino acids or chemical amino acid analogs can be introduced as a substitution or addition into the particular TCAP sequence. Non-classical amino acids include, but are not limited, to the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general. Furthermore, the amino acid can be D (dextrorotary) or L (levorotary).

In a specific embodiment, the derivative of a particular TCAP is a chimeric, or fusion, protein comprising a TCAP protein or fragment thereof, preferably consisting of at least a domain or motif of the particular TCAP, or at least 6 amino acids of the particular TCAP, joined at its amino- or carboxy-terminus via a peptide bond to an amino acid sequence of a different protein. In one embodiment, such a chimeric protein is produced by recombinant expression of a nucleic acid encoding the protein, comprising a TCAP-coding sequence joined in-frame to a coding sequence for a different protein. Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g. by use of a peptide synthesizer. Chimeric genes comprising portions of a TCAP gene, fused to any heterologous protein-encoding sequences, may be constructed. A specific embodiment relates to a chimeric protein comprising a fragment of a particular TCAP of at least six amino acids.

Other specific embodiments of derivatives are described in the subsection below and examples sections infra.

### 5.6.1. DERIVATIVES OF PARTICULAR TCAPS CONTAINING ONE OR MORE DOMAINS OF THE PROTEIN

In a specific embodiment, the invention relates to TCAP derivatives, in particular derivatives of TA-GAP, TA-GPCR, TA-PP2C, TA-NFKBH, TA-KRP, TA-WDRP, or TA-LRRP, including TA-GAP RhoGAP domain, TA-GPCR extracellular, transmembrane, intracellular or GTP-binding domains, TA-WDRP GPCR-binding or WD motif-containing domain, TA-WDRP or TA-KRP β propeller domains kelch repeats and POZ/BTB domain; TA-NFKBH ankyrin repeats and DNA-binding domains and TA-LRRP transmembrane domains and leucine-rich repeat domains; and fragments and derivatives of such fragments, that comprise, or alternatively consist of, one or more domains of a TCAP including but not limited to a functional (e.g., binding) fragment of any of the foregoing, or any combination of the foregoing TCAPs.

In another specific embodiment, a molecule is provided that comprises one or more domains (or functional portion thereof) of a particular TCAP protein but that also lacks one or more domains (or functional portion thereof) of that particular TCAP. In particular examples, TA-GPCR derivatives are provided that lack an intracellular, GTP-binding, or transmembrane domain. By way of another example, such a TA-GPCR may also lack all or a portion of the extracellular domain, but retain at least the transmembrane or intracellular domains of a TA-GPCR. In another embodiment, a molecule is provided that comprises one or more domains (or functional portion thereof) of a TCAP and that has one or more mutant (e.g., due to deletion or point mutation(s)) domains of a TCAP such that the mutant domain has increased or decreased function. By way of example, the TA-GPCR extracellular domain may be mutated so as to have reduced, absent, or increased ligand-binding activity. A person of skill in the art would understand that fragments comprising one or more domains, or one or more mutant domains, may be derived from other TCAPs, as well. In a specific embodiment, one, two, or three point mutations are present.

### 5.7. UTILITY

The invention provides TCAPs having useful activities. The invention further provides the use of TCAPs or derivatives thereof, TCAP nucleic acids, and antibodies that recognize TCAPs, or derivatives thereof, as markers for the activation, or lack thereof, of T cells. Such markers enable the screening for diagnosis, staging and monitoring of therapies of diseases and disorders associated with undesirable T cell activation, or, alternatively, where T cell insufficient T cell activation has occurred. For example, the invention provides monitoring of therapies directed to the suppression of inappropriate or undesired T cell activation, or of therapies directed to the enhancement of T cell activation, where such activation is desired. Finally, the invention provides for the use of TCAPs or derivatives thereof, TCAP nucleic acids, or antibodies that recognize TCAPs, or derivatives thereof, as therapeutic agents for the treatment of conditions related to T cell activation or lack thereof.

### 5.7.1. USEFUL ACTIVITIES ASSOCIATED WITH TCAPs

The TCAPs of the present invention have activities useful in their own right. These activities may be used in vitro to accomplish desired reactions. They may also be used as part of in vitro models of the particular biochemical system of which they are a part. Each may also be used as a target for immunomodulatory drugs, wherein the immunomodulatory drug enhances or, more generally, represses, the activity of a particular TCAP. Such immonoregulatory effect is established either directly by showing an effect on T cell activation when applied to model T cells, for example, Jurkat T cells, or indirectly by showing a modulation of the transcription of one or more TCAP genes, or of the activity of one or more TCAPs. The utility of each TCAP described herein is discussed in more detail below.

### 5.7.1.1. TA-GAP

GAPs have the intrinsic activity of stimulating the GTPase activity of GTPases. This activity is useful in assays of GTPase activity, particularly Rho GTPase activity, on G protein-mediated signaling pathways. Assays for Rho GAP activity have been described (Toure et al., J. Biol. Chem. 273(11):6019-6023 (1998); Ross & Wilkie, Ann. Rev. Biochem. 69:795-827 (2000)). Furthermore, because of the control exerted by GTPases, and therefore, by GAPs, over cell growth and proliferation, GAPs are also natural targets for drug discovery. Several GAPs have been described as useful in the diagnosis and treatment of cancers. See Weissbach et al., U.S. Pat. No. 5,639,651; Wong et al., U.S. Pat. No. 5,760,203. Thus, TA-GAP is highly likely to be useful not only for its intrinsic GTPase-regulating activity, but as a target for drugs directed to the suppression of T cell activation and proliferation.

### 5.7.1.2. TA-GPCR

The useful activity of a GPCR is its ability to transmit extracellular signals to the interior of the cell. As a consequence of relatively small ligand-binding sites and the wide range of physiological events which they regulate, GPCRs have well-known utility as targets for drugs; in fact, GPCRs constitute the largest class of drug targets in humans (Flower, Biochim. et Biophys. Acta. 1422:207-234 (1999)). In fact, existing studies of GPCRs have established a pattern for drug discovery that any new drug discovery project might reasonably follow. When the sequence for a new GPCR is determined, comparison of the sequence to existing GPCRs with known functions enables one to determine the broad features of the binding site, which, in turn, suggests the types of compounds that may be made or selected from a compound bank or commercial database to interact with that binding site. See Flower, supra. Thus, TA-GPCR is useful as a target for drug studies, where the drug in question is to modulate T cell activation. A number of GPCRs have been described as useful in a variety of diagnostic and/or therapeutic applications. See, e.g., MacLennan, U.S. Pat. No. 5,585,476; Soppet et al., U.S. Pat. No. 5,756,309; Soppet et al., U.S. Pat. No. 5,776,729. Methods for assaying for GPCR activity have been described previously (Sadee, U.S. Pat. No. 5,882,944; Barak et al., U.S. Pat. No. 5,891,646).

### 5.7.1.3. TA-WDRP

G proteins function to transmit signals received by GPCRs to enzymes that create effector molecules, such as cAMP, inositol triphosphate, and phospholipase C. The useful activity of G proteins thus lies in their place in signal transduction, and on this basis, like GPCRs, they have been drug targets. See, e.g., Doll et al., U.S. Pat. No. 6,214,828 (describing compounds directed to G proteins useful in reducing cell proliferation).

### 5.7.1.4. TA-NFKBH

The useful activity of TA-NFKBH is its ability to promote the transcription of genes. Thus, TA-NFKBH represents another potential target for drug therapies directed to the modulation of T cell activation. As noted in Section 2.5, the inappropriate regulation of NF-κB and its dependent genes has been associated with septic shock, graft-versus-host disease, acute inflammatory conditions, acute phase response, transplant rejection, autoimmune diseases, and cancer (Manna & Agarwal, J. Immunol. 165:2095-2102 (1999)); as TA-NFKBH is produced during T cell activation, it is highly likely that the genes whose transcription is promoted by TA-NFKBH are similarly involved in these conditions. NF-κB has also been described as an attractive and highly useful target for therapies directed to these conditions, including small molecule or antisense inhibition. See, e.g., Narayanan et al., U.S. Pat. No. 5,591,840. For example, one agent, known as A77 1726, exhibits antiinflammatory, antiproliferative and immunosuppressive effects by blocking TNF-dpendent NF-κB activation and gene expression (Manna & Agarwal, above). Based on the sequence homology of TA-NFKBH to NF-κB, it is likely that TA-NFKBH is similarly useful as a target for antiinflammatory and immunosuppressive drugs.

### 5.7.1.5. TA-PP2C

Based on sequence homologies, TA-PP2C is a class 2C phosphatase (a PP2C) and possesses serine/threonine phosphatase activity, that is, the ability to remove phosphates from serine or threonine residues. This activity is useful in any assay that involves the kinasing of serine or threonine residues, to reverse the kinasing reaction. Assays for PP2Cs have been described (Cheng et al., J. Biol. Chem. 274(44):34733-34749 (2000); Takekawa et al., EMBO J. 17:4744-4752 (1998)). Thus, TA-PP2C has utility for its intrinsic enzymatic activity. Moreover, TA-PP2C can be used to identify inhibitors of serine/threonine phosphatase activity in vitro; such assays have been described (Matsuzawa et al., FEBS Lett. 19:356(2-3):272-4 (1994).

### 5.7.1.6. ASSAYS OF TCAPS AND TCAP DERIVATIVES

In addition to the specific assays referenced above, the functional activity of TCAPs, derivatives can be assayed by various other methods. For example, in one embodiment, where one is assaying for the ability to bind or compete with the wild-type of a particular TCAP for binding to an antibody raised against the protein, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In another embodiment, in those situations where a TCAP-binding protein is identified, the binding can be assayed, e.g., by means well-known in the art. In another embodiment, physiological correlates of the binding of a TCAP to its substrate(s) can be assayed.

In another embodiment, in insect or other model systems, genetic studies can be done to study the phenotypic effect of a TCAP mutant that is a derivative of wild-type TCAP.

In addition, assays that can be used to detect or measure the ability to inhibit, or alternatively promote the activities of the TCAPs described herein, are described in Section 5.7.4.

Other methods will be known to the skilled artisan and are within the scope of the invention.

### 5.7.2. TCAPS AS MARKERS OF T CELL ACTIVATION

The TCAPs of the present invention are proteins specifically produced during T cell activation. Thus, these proteins, or the associated nucleic acids, in abundances exceeding that of the normal state (i.e., wherein T cells are not substantially activated), are markers of T cell activation. As such, they are useful markers for any condition for which the monitoring of the state of T cell activation is desirable. Thus, measuring one or more of the TCAPs or TCAP nucleic acids (e.g., mRNA, cDNA or cRNA) in a cell sample can be used to assess whether a person suffers a condition associated with increased T cell activation, where T cell activation is undesirable, or lack of T cell activation, where T cell activation is desirable. A number of immune-related disorders or conditions, such as autoimmune disorders or severe combined immune disorder involve the undesirable activation of T cells. Many physiological, pathological or therapeutic conditions also involve T cell activation, such as bacterial, viral or organismal infections, and responses thereto, vaccinations and responses thereto, allergies and allergic reactions, immune therapies, transplants, and graft-versus-host disease. Conversely, some physiological, pathological or therapeutic conditions involve insufficient T cell activation, where T cell activation is desirable, such as acquired immune deficiency syndrome or chemotherapy. In a hospital, clinical or research setting, the ability to easily track the response of the immune system to various therapies, and to easily assess the immune status of a patient, would be a highly useful component of any course of treatment directly or indirectly affecting or involving the immune system.

The present invention, therefore, provides markers of T cell activation useful for assessing the immune status of a person. Specifically, the invention provides for the use of the TCAPs TA-GPCR, TA-GAP, TA-WDRP, TA-NFKBH, TA-KRP,TA-WDRP and/or, TA-LRRP and the nucleic acids encoding them, as markers of T cell activation. These markers will assist in determining the efficacy of immune-suppressive therapies, for example, to monitor the effectiveness of drugs used to prevent graft-versus-host disease or of treatments for allergies or the suppression of the allergic response. The markers will also assist in monitoring the effectiveness of immune-promoting therapies, for example, certain vaccines, AIDS therapies, or SCID therapies.

The use of TCAPs as markers is straightforward. First, antibodies to one or more TCAPs are raised or obtained according to the methods presented in Section 5.5. These antibodies are then used in an immunoassay to detect a particular TCAP, which immunoassay is carried out by a method comprising contacting a sample derived from a patient with the anti-TCAP antibody under conditions such that immunospecific binding can occur, and detecting or measuring the amount of any immunospecific binding by the antibody. In a specific aspect, such binding of antibody, in tissue sections or in patient samples, can be used to detect aberrant localization or aberrant (e.g., low, absent, or high) levels of a particular TCAP. In a specific embodiment, antibody(ies) to one or more TCAP can be used to assay in a patient tissue or serum sample for the presence of TCAP where an aberrant level of TCAP is an indication of a diseased condition. “Aberrant level” means an increased or decreased level relative to that present, or a standard level representing that present, in an analogous sample from a portion of the body or from a subject not having the disorder. In another specific embodiment, antibody(ies) to one or more TCAP can be used to assay in a patient tissue or serum sample increased or decreased levels of the TCAP(s) to assess the efficacy, stage, or progress of an immune system-promoting or immunosuppressive therapy, respectively.

The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, etc.

In similar fashion, mRNA encoding a particular TCAP can act as a marker for T cell activation, and, therefore, can be used in the same manner as TCAPs and antibodies to TCAPs. In this regard, RNA is extracted from a sample and is used in an assay capable of detecting the presence and amount of RNA present in a sample, such as northern analysis, slot blots, microarray analysis, quantitative PCR, etc. TCAP-encoding nucleic acid sequences, or subsequences thereof comprising about at least eight (8) nucleotides, including complementary sequences, can be used as hybridization probes. Hybridization assays can be used to detect, prognose, diagnose, or monitor conditions, disorders, or disease states associated with aberrant changes in TCAP expression and/or activity as described supra. In particular, such a hybridization assay is carried out by a method comprising contacting a sample containing nucleic acid with a nucleic acid probe capable of hybridizing to TCAP mRNA, or nucleic acid derived therefrom, under conditions such that hybridization can occur, and detecting or measuring any resulting hybridization. In a specific embodiment, nucleic acids derived from TCAP mRNA, such as cDNA or cRNA, are measured. As used herein, cRNA is defined here as RNA complementary to the source RNA or its complement, i.e., complementary to either strand of a cDNA of the source RNA. The extracted RNAs are preferably amplified using a process in which doubled-stranded cDNAs are synthesized from the RNAs using a primer linked to an RNA polymerase promoter in a direction capable of directing transcription of anti-sense RNA. Anti-sense RNAs or cRNAs are then transcribed from the second strand of the double-stranded cDNAs using an RNA polymerase (see, e.g., U.S. Pat. Nos. 5,891,636, 5,716,785; 5,545,522 and 6,132,997). Both oligo-dT primers (U.S. Pat. Nos. 5,545,522 and 6,132,997) or random primers (U.S. Provisional Patent Application Serial No. 60/253,641) that contain an RNA polymerase promoter or complement thereof can be used. Preferably, the target polynucleotides are short and/or fragmented polynucleotide molecules which are representative of the original nucleic acid population of the cell. In a most preferred embodiment, the nucleic acid probe is one of a plurality of different probes on a microarray.

Collection of a sample from a patient can be by any means known in the art. For example, because T cells are blood cells, a patient sample can comprise a blood, serum, or plasma sample. In a specific embodiment, the sample comprises peripheral blood mononuclear cells (PBMCs). The sample may also comprise a tissue sample, drawn from a site of inflammation. Tissue can be biopsied or derived from any organ of the body affected, including bone and skin. Tissue can be obtained surgically or by fine needle aspiration.

Typically, blood, serum, plasma or tissue samples from which RNA is to be extracted are quick frozen on dry ice. Samples are then homogenized together with a mortar and pestle under liquid nitrogen. A typical RNA extraction procedure is as follows. Total cellular RNA is extracted from tissue with either RNAzol™ or RNAzolB™ (Tel-Test, Friendswood, Tex.), according to the manufacturer's instructions. The tissue is solubilized in an appropriate amount of RNAzol™ or RNAzolB™, and RNA is extracted by the addition of 1/10 v/v chloroform to the solubilized sample followed by vigorous shaking for approximately 15 seconds. The mixture is then centrifuged for 15 minutes at 12,000 g and the aqueous phase removed to a fresh tube. RNA is then precipitated with isopropanol. The resultant RNA pellet is dissolved in water and re-extracted with an equal volume of chloroform to remove any remaining phenol. The extracted volume is precipitated with 2 volumes of ethanol in the presence of 150 mM sodium acetate. The precipitated RNA is then dissolved in water and the concentration determined spectroscopically (A260).

In specific embodiments, diseases and disorders involving reduced activation of T cells can be diagnosed, or their suspected presence can be screened for, or a predisposition to develop such disorders can be detected, by detecting decreased levels of TCAP protein, TCAP RNA, or TCAP functional activity (e.g., phosphatase activity, SH3 domain-binding activity, GTPase activity, ligand-binding activity, transcriptional activation activity, etc.), or by detecting mutations in TCAP RNA, DNA or protein (e.g., translocations of a TCAP nucleic acid, truncations in a TCAP gene or protein, changes in nucleotide or amino acid sequence relative to wild-type TCAP) that cause decreased expression or activity of TCAP. Such diseases and disorders include but are not limited to immune function reduction or failure resulting from chemotherapy, HIV infection, septic shock, or severe combined immune deficiency. By way of example, reduced levels of a particular TCAP, in comparison to a normal or control sample, can be detected by immnunoassay; levels of TCAP RNA can be detected by hybridization assays (e.g., Northern blots, dot blots); the activity of a particular TCAP can be measured using assays known in the art; translocations and point mutations in TCAP nucleic acids can be detected by Southern blotting, RFLP analysis, PCR using primers that preferably generate a fragment spanning at least most of a TCAP gene, sequencing of the TCAP genomic DNA or cDNA obtained from the patient; etc. Where levels of TCAPs, TCAP nucleic acid, or TCAP activity are to be measured, in some instances no TCAP, TCAP nucleic acid, or TCAP activity can be discerned in a sample, as compared to a normal or control sample. In this instance, the absence of the TCAP, TCAP nucleic acid or TCAP activity indicates the presence of a disease or disorder involving the reduced activation of T cells.

In one embodiment, levels of TCAP mRNA or protein in a patient sample are detected or measured, in which decreased levels indicate that the subject has, or has a predisposition to developing, a disorder involving underactivation of T cells; in which the decreased levels are relative to the levels present in an analogous sample from a not having such a disorder.

In another embodiment, diseases and disorders involving undesirable T cell proliferation, or in which T cell activation and/or proliferation is desirable for treatment, are diagnosed, or their suspected presence can be screened for, or a predisposition to develop such disorders can be detected, by detecting increased levels of a particular TCAP, or the RNA encoding the particular TCAP, or the functional activity of a particular TCAP (e.g., phosphatase activity, GTPase activity, GTPase activation activity, transcriptional activation activity, transducin-like activity, etc.), or by detecting mutations in the RNA, DNA or amino acid sequence of a particular TCAP (e.g., translocations in TCAP nucleic acids, truncations in the gene or protein, changes in nucleotide or amino acid sequence relative to wild-type TCAP) that cause increased expression or activity of a particular TCAP. Such diseases and disorders include but are not limited to graft-versus-host disease, allergic reactions, undesirable reactions to vaccinations, or autoimmune disorders in which the immune system recognizes a component of the body. By way of example, levels of TCAP protein, levels of TCAP RNA, TCAP kinase activity, TCAP binding activity, and the presence of translocations or point mutations can be determined as described above.

In another embodiment, levels of TCAP nucleic acid or protein in a patient sample are detected or measured, in which increased levels indicate that the subject has, or has a predisposition to developing, a T cell activation disorder in which the increased levels are relative to the levels present in an analogous sample from a portion of the body or from a subject not having the disorder.

Kits for diagnostic use are also provided that comprise in one or more containers an anti-TCAP antibody, and, optionally, a labeled binding partner to the antibody. Alternatively, the anti-TCAP antibody can be labeled with a detectable-moiety, e.g., a chemiluminescent, enzymatic, fluorescent, or radioactive moiety. A kit is also provided that comprises in one or more containers a nucleic acid probe capable of hybridizing to TCAP RNA. In a specific embodiment, a kit can comprise in one or more containers a pair of primers (e.g., each in the size range of 6-30 nucleotides) that are capable of priming amplification, e.g., by polymerase chain reaction (PCR; Innis et al., PCR Protocols, Academic Press, Inc., San Diego, Calif. (1990)), ligase chain reaction (EP 320,308) use of Q replicase, cyclic probe reaction, or other methods known in the art, under appropriate reaction conditions of at least a portion of a TCAP-encoding nucleic acid. A kit can optionally further comprise in a container a predetermined amount of a purified TCAP protein or nucleic acid, e.g., for use as a standard or control, and/or a container comprising a buffer in which PCR or another amplification reaction can be conducted, and/or a container comprising an enzyme (e.g., a polymerase) suitable for use in the amplification reaction.

### 5.7.3. THERAPEUTIC USES

### 5.7.3.1. GENE THERAPY

The invention also provides for treatment of various diseases and disorders by administration of a therapeutic compound (termed herein “Therapeutic”). Such “Therapeutics” include, but are not limited to: TCAPs and derivatives (including fragments) thereof (e.g., as described herein above); antibodies thereto (as described herein above); nucleic acids encoding the particular TCAP(s) or TCAP derivatives (e.g., as described herein above); antisense nucleic acids to nucleic acids encoding a particular TCAP, and agonists and antagonists. Disorders involving under-activation of T cells are treated by administration of a Therapeutic that promotes the function of a particular TCAP or set of TCAPs. Where T cell activity is sought to be reduced, e.g., in immunosuppressive therapy, reduction is accomplished by administration of a Therapeutic that antagonizes (inhibits) the function of a TCAP or set of TCAPs. The above is described in detail in the subsections below.

Generally, administration of products of a species origin or species reactivity (in the case of antibodies) that is the same species as that of the patient is preferred. Thus, in a preferred embodiment, a human TCAP, derivative, or nucleic acid, or an antibody to a human TCAP, is therapeutically or prophylactically administered to a human patient.

In a specific embodiment, the invention further provides a method of treating or preventing a disease or disorder involving undesirable T cell activation in a subject comprising administering to a subject in which such treatment is desired a therapeutically effective amount of a molecule that inhibits the function of at least one TCAP. In a more specific embodiment, the subject is a human. In a more specific embodiment, the invention provides the method above, wherein the molecule that inhibits TCAP function (i.e., the therapeutic) is selected from the group consisting of a TCAP derivative that is active in inhibiting cell proliferation, a nucleic acid encoding a TCAP, a nucleic acid encoding a TCAP derivative that is active in inhibiting cell proliferation, an anti-TCAP antibody or a fragment or derivative thereof containing the binding region thereof, a nucleic acid complementary to the RNA produced by transcription of a TCAP gene, and a nucleic acid comprising at least a portion of a TCAP gene into which a heterologous nucleotide sequence has been inserted such that said heterologous sequence inactivates the biological activity of the at least a portion of the TCAP gene, in which the TCAP gene portion flanks the heterologous sequence so as to promote homologous recombination with a genomic TCAP gene. In a further, more specific embodiment of the method above, the therapeutic that inhibits TCAP function is an oligonucleotide that (a) consists of at least six nucleotides; (b) comprises a sequence complementary to at least a portion of an RNA transcript of a TCAP gene; and (c) is hybridizable to the RNA transcript under moderately stringent conditions. In yet another specific embodiment of the above method, the molecule that inhibits TCAP function is a protein having at least 60% identity to a domain of a TCAP.

The invention further provides a method of treating a disease or disorder involving a deficiency in cell proliferation or in which cell proliferation is desirable for treatment in a subject comprising administering to a subject in which such treatment is desired a therapeutically effective amount of a molecule that promotes TCAP function.

In a specific embodiment, nucleic acids comprising a sequence encoding a TCAP or functional derivative thereof, are administered to promote TCAP function, by way of gene therapy. Gene therapy refers to therapy performed by the administration of a nucleic acid to a subject. In this embodiment of the invention, the nucleic acid produces its encoded protein that mediates a therapeutic effect by promoting TCAP function.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993)). Methods commonly known in the art of recombinant DNA technology which can be used are described in Ausubel et al. (eds.), Current Protocols in Molecular Biology, John Wiley C Sons, NY (1993); and Kriegler, Gene Transfer and Expression, A Laboratory Manual, Stockton Press, NY (1990).

In a preferred aspect, the Therapeutic comprises a TCAP-encoding nucleic acid that is part of an expression vector that expresses a TCAP protein or fragment or chimeric protein thereof in a suitable host. In particular, such a nucleic acid has a promoter operably linked to the TCAP gene coding region, said promoter being inducible or constitutive, and, optionally, tissue-specific. In another particular embodiment, a nucleic acid molecule is used in which the TCAP coding sequences and any other desired sequences are flanked by regions that promote homologous recombination at a desired site in the genome, thus providing for intrachromosomal expression of the TCAP nucleic acid (Koller and Smithies, Proc. Natl. Acad. Sci. U.S.A. 86:8932-8935 (1989); Zijlstra et al., Nature 342:435-438 (1989)).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), or by direct injection of naked DNA, or by use of microparticle bombardment (e.g., a gene gun; Biolistic, DuPont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., Wu and Wu, Biol. Chem. 262:4429-4432 (1987)) (which can be used to target cell types specifically expressing the receptors), etc. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 published Apr. 16, 25, 1992 (Wu et al.); WO 92/22635 published Dec. 23, 1992 (Wilson et al.); WO92/20316 published Nov. 26, 1992 (Findeis et al.); WO93/14188 published Jul. 22, 1993 (Clarke et al.), WO 93/20221 published Oct. 14, 1993 (Young)). Alternatively, the nucleic acid can be introduced intracellularly and incorporated within host cell DNA for expression, by homologous recombination (Koller and Smithies, Proc. Natl. Acad. Sci. U.S.A. 86:8932-8935 (1989); Zijlstra et al., Nature 342:435-438 (1989)).

In a specific embodiment, a viral vector that contains the TCAP-encoding nucleic acid is used. For example, a retroviral vector can be used (see Miller et al., Meth. Enzymol. 217:581-599 (1993)). These retroviral vectors have been modified to delete retroviral sequences that are not necessary for packaging of the viral genome and integration into host cell DNA. The TCAP-encoding nucleic acid to be used in gene therapy is cloned into the vector, which facilitates delivery of the gene into a patient. More detail about retroviral vectors can be found in Boesen et al., Biotherapy 6:291-302 (1994), which describes the use of a retroviral vector to deliver the mdrl gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrate the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); and Mastrangeli et al., J. Clin. Invest. 91:225-234 (1993).

Adeno-associated virus (AAV) has also been proposed for use in gene therapy (Walsh et al., Proc. Soc. Exp. Biol. Med. 204:289-300 (1993).

Another approach to gene therapy involves transferring a gene to cells in tissue culture. The expression of the transferred gene may be controlled by its native promoter, or can be controlled by a non-native promoter (see Section 5.2, supra; Section 5.7.3.1, infra). In addition to transferring a nucleic acid comprising a nucleic acid sequence encoding an entire TCAP (i.e., equivalent to the wild type), the transferred nucleic acids can encode a functional portion of a particular TCAP, or a protein having at least 60% sequence identity to a TCAP disclosed herein, as compared over the length of the particular TCAP, or protein (whichever is shorter) or a polypeptide having at least 60% sequence similarity to a TCAP fragment, as compared over the length of the TCAP fragment or polypeptide (whichever is shorter). Introduction of the nucleic acid into the cell is accomplished by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see e.g., Loeffler and Behr, Meth. Enzymol. 217:599-618 (1993); Cohen et al., Meth. Enzymol. 217:618-644 (1993); Cline, Pharmac. Ther. 29:69-92 (1985)) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. In a preferred embodiment, epithelial cells are injected, e.g., subcutaneously. In another embodiment, recombinant skin cells may be applied as a skin graft onto the patient. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, a TCAP-encoding nucleic acid is introduced into the cells such that it is expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention. Such stem cells include but are not limited to hematopoietic stem cells (HSC), stem cells of epithelial tissues such as the skin and the lining of the gut embryonic heart muscle cells, liver stem cells (PCT Publication WO 94/08598, published Apr. 28, 1994), and neural stem cells (Stemple and Anderson, Cell 71:973-985 (1992)).

Epithelial stem cells (ESCs) or keratinocytes can be obtained from tissues such as the skin and the lining of the gut by known procedures (Rheinwald, Meth. Cell Bio. (21A):229 (1980)). In stratified epithelial tissue such as the skin, renewal occurs by mitosis of stem cells within the germinal layer, the layer closest to the basal lamina. Stem cells within the lining of the gut provide for a rapid renewal rate of this tissue. ESCs or keratinocytes obtained from the skin or lining of the gut of a patient or donor can be grown in tissue culture (Rheinwald, Meth. Cell Bio. 21A:229 (1980); Pittelkow and Scott, Mayo Clinic Proc. 61:771 (1986)). If the ESCs are provided by a donor, a method for suppression of host versus graft reactivity (e.g., irradiation, drug or antibody administration to promote moderate immunosuppression) can also be used.

With respect to hematopoietic stem cells (HSC), any technique which provides for the isolation, propagation, and maintenance in vitro of HSC can be used in this embodiment of the invention. Techniques by which this may be accomplished include (a) the isolation and establishment of HSC cultures from bone marrow cells isolated from the future host, or a donor, or (b) the use of previously established long-term HSC cultures, which may be allogeneic or xenogeneic. Non-autologous HSC are used preferably in conjunction with a method of suppressing transplantation immune reactions of the future host/patient. In a particular embodiment of the present invention, human bone marrow cells can be obtained from the posterior iliac crest by needle aspiration (see, e.g., Kodo et al., J. Clin. Invest. 73:1377-1384 (1984)). In a preferred embodiment of the present invention, the HSCs can be made highly enriched or in substantially pure form. This enrichment can be accomplished before, during, or after long-term culturing, and can be done by any techniques known in the art. Long-term cultures of bone marrow cells can be established and maintained by using, for example, modified Dexter cell culture techniques (Dexter et al., J. Cell Physiol. 91:335 (1977)) or Witlock-Witte culture techniques (Witlock and Witte, Proc. Natl. Acad. Sci. U.S.A. 79:3608-3612 (1982)).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

### 5.7.3.1.1. ANTISENSE REGULATION OF EXPRESSION OF TCAP GENES

In a specific embodiment, the function of a particular TCAP is inhibited by use of antisense nucleic acids substantially complementary to the transcript from a TCAP-encoding gene. The present invention provides the therapeutic or prophylactic use of nucleic acids of at least six nucleotides that are antisense to a gene or cDNA encoding TCAP or a portion thereof. A “TCAP antisense nucleic acid” as used herein refers to a nucleic acid that of hybridizes to a sequence-specific nucleic acid (preferably mRNA) segment (i.e., not the poly-A tract of an mRNA) that encodes TA-GPCR, TA-GAP, TA-NFKBH, TA-KRP, TA-PP2C, TA-WDRP or TA-LRRP by virtue of some sequence complementarity. The antisense nucleic acid may be complementary to a coding and/or noncoding region of an mRNA encoding these TCAPs. Such antisense nucleic acids have utility as Therapeutics that inhibits TCAP function, and can be used in the treatment of disorders that result from T cell activation.

The antisense nucleic acids of the invention can be oligonucleotides that are double-stranded or single-stranded, RNA or DNA or a modification or derivative thereof, which can be directly administered to a cell, or which can be produced intracellularly by transcription of exogenous, introduced sequences.

The invention further provides pharmaceutical compositions comprising an effective amount of the TCAP antisense nucleic acids of the invention in a pharmaceutically acceptable carrier, as described infra.

In another embodiment, the invention is directed to methods for inhibiting the expression of a TCAP-encoding nucleic acid sequence in a prokaryotic or eukaryotic cell comprising providing the cell with an effective amount of a composition comprising a TCAP antisense nucleic acid of the invention.

TCAP antisense nucleic acids and their uses are described in detail below.

### 5.7.3.1.2. TCAP ANTISENSE NUCLEIC ACIDS

The TCAP antisense nucleic acids of the present invention are of at least six nucleotides and are preferably oligonucleotides (typically ranging from 6 to about 50 oligonucleotides). In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof, single-stranded or double-stranded. The oligonucleotide can be modified at the base moiety, sugar moiety, or phosphate backbone. The oligonucleotide may include other appending groups such as peptides, or agents facilitating transport across the cell membrane (see, e.g., Letsinger et al., Proc. Natl. Acad. Sci. U.S.A. 86:6553-6556 (1989); Lemaitre et al., Proc. Natl. Acad. Sci. U.S.A. 84:648-652 (1987); PCT Publication No. WO 88/09810, published Dec. 15, 1988) or blood-brain barrier (see, e.g., PCT Publication No. WO 89/10134, published Apr. 25, 1988), hybridization-triggered cleavage agents (see, e.g., Krol et al., BioTechniques 6:958-976 (1988)) or intercalating agents (see, e.g., Zon, Pharm. Res. 5:539-549 (1988)).

In a preferred aspect of the invention, a TCAP antisense oligonucleotide is provided, preferably of single-stranded DNA. In a most preferred aspect, such an oligonucleotide comprises a sequence antisense to the sequence encoding one or more domains of a TCAP protein, most preferably, of a human TCAP protein. The oligonucleotide may be modified at any position on its structure with substituents generally known in the art.

The TCAP antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, 5 beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In another embodiment, the oligonucleotide comprises at least one modified sugar moiety selected from the group including but not limited to arabinose, 2-fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a thiophosphoamidate, a phosphordiamidate, a methylphosphonate, an alkyl phosphotriester, and a formacetal or analog thereof.

In yet another embodiment, the oligonucleotide is an α-anomeric oligonucleotide. An a-anomeric oligonucleotide forms specific double-stranded hybrids with complementary RNA in which, contrary to the usual β-units, the strands run parallel to each other (Gautier et al., Nucl. Acids Res. 15:6625-6641 (1987)).

The oligonucleotide may be conjugated to another molecule, e.g., a peptide, hybridization triggered cross-linking agent, transport agent, hybridization-triggered cleavage agent, etc.

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. Nucl. Acids Res. 16:3209 (1988), methylphosphonate oligonucleotides can be prepared by use of controlled pore glass polymer supports (Sarin et al., Proc. Natl. Acad. Sci. U.S.A. 85:7448-7451 (1988)), etc.

In a specific embodiment, the TCAP antisense oligonucleotide comprises catalytic RNA, or a ribozyme (see, e.g., PCT International Publication WO 90/11364, published Oct. 4, 1990; Sarver et al., Science 247:1222-1225 (1990)). In another embodiment, the oligonucleotide is a 2′-O-methylribonucleotide (Inoue et al., Nucl. Acids Res. 15:6131-6148 (1987)), or a chimeric RNA-DNA analog (Inoue et al., FEBS Lett. 215: 327-330 (1987)).

In an alternative embodiment, the TCAP antisense nucleic acid of the invention is produced intracellularly by transcription from an exogenous sequence. For example, a vector can be introduced in vivo such that it is taken up by a cell, within which cell the vector or a portion thereof transcribed, producing an antisense nucleic acid (RNA) of the invention. Such a vector would contain a sequence encoding the TCAP antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the TCAP antisense RNA can be by any promoter known in the art to act in mammalian, preferably human, cells. Such promoters can be inducible or constitutive. Such promoters include but are not limited to: the SV40 early promoter region (Bernoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. U.S.A. 78:1441-1445 (1981)), the regulatory sequences of the metallothionein gene (Brinster et al., Nature 296:39-42 (1982)), etc.

The antisense nucleic acids of the invention comprise a sequence complementary to at least a portion of a “RNA transcript of a TCAP gene, preferably a human TCAP gene. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded TCAP antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA transcribed from a TCAP-encoding gene it may contain and still form a stable duplex (or triplex, as the case may be). One skilled in the art can ascertain a tolerable degree of mismatch by use of standard procedures to determine the melting point of the hybridized complex. The antisense nucleic acids of the present invention hybridize to the target nucleic acid under moderately stringent conditions, and more preferably hybridize under highly stringent conditions.

### 5.7.3.1.3. THERAPEUTIC USE OF ANTISENSE NUCLEIC ACIDS TO TCAP-ENCODING GENES

Antisense nucleic acids to the TCAP-encoding genes of the present invention can be used to treat disorders of a cell type that expresses, or preferably overexpresses, the particular TCAP to which the antisense nucleic acid is directed. In a specific embodiment, such a disorder is a hyperactivation of the immune system mediated by T cells. In more specific embodiment, such a disorder is an immune system disorder that results in, or is attributable to, the overexpression of TA-GPCR, TA-GAP, TA-NFKBH, TA-KRP, TA-PP2C, TA-WDRP or TA-LRRP. In a preferred embodiment, a single-stranded DNA antisense TCAP oligonucleotide is used.

Cell types which express or overexpress TCAP RNA can be identified by various methods known in the art. Such methods include but are not limited to hybridization with a TCAP-specific nucleic acid (e.g. by Northern hybridization, dot blot hybridization, in situ hybridization), observing the ability of RNA from the cell type to be translated in vitro into qTCAP, immunoassay, etc. In a preferred aspect, primary tissue from a patient can be assayed for expression one or more TCAP prior to treatment, e.g., by immunocytochemistry or in situ hybridization.

Pharmaceutical compositions of the invention (see Section 5.7.3.3), comprising an effective amount of a TCAP antisense nucleic acid in a pharmaceutically acceptable carrier, can be administered to a patient having a disease or disorder which is of a type that expresses or overexpresses a TCAP or TCAP RNA.

The amount of TCAP antisense nucleic acid which will be effective in the treatment of a particular disorder or condition will depend on the nature of the disorder or condition, and can be determined by standard clinical techniques. Where possible, it is desirable to determine the antisense cytotoxicity of the tumor type to be treated in vitro, and then in useful animal model systems prior to testing and use in humans.

In a specific embodiment, pharmaceutical compositions comprising TCAP antisense nucleic acids are administered via liposomes, microparticles, or microcapsules. In various embodiments of the invention, it may be useful to use such compositions to achieve sustained release of the TCAP antisense nucleic acids. In a specific embodiment, it may be desirable to utilize liposomes targeted via antibodies to specific identifiable tumor antigens (Leonetti et al., 1990, Proc. Natl. Acad. Sci. U.S.A. 87:2448-2451 (1990); Renneisen et al., J. Biol. Chem. 265:16337-16342 (1990)).

### 5.7.3.2. DEMONSTRATION OF THERAPEUTIC OR PROPHYLACTIC UTILITY

The Therapeutics of the invention are preferably tested in vitro, and then in vivo for the desired therapeutic or prophylactic activity, prior to use in humans.

For example, in vitro assays which can be used to determine whether administration of a specific Therapeutic is indicated, include in vitro cell culture assays in which a patient tissue sample is grown in culture, and exposed to or otherwise administered a Therapeutic, and the effect of such Therapeutic upon the tissue sample is observed. In one embodiment, a Therapeutic that reverses or reduces the activation of T cells is selected for therapeutic use in vivo. Many assays standard in the art can be used to assess such survival and/or growth; for example, cell proliferation can be assayed by measuring 3H-thymidine incorporation, by direct cell count, by detecting changes in transcriptional activity of known genes such as proto-oncogenes (e.g., fos, myc) or cell cycle markers; cell viability can be assessed by trypan blue staining, differentiation can be assessed visually based on changes in morphology, etc.

In another embodiment, a Therapeutic is indicated for use which exhibits the desired effect, inhibition or promotion of T cell activation, upon a patient sample where the patient suffers a condition associated with T cell activation.

In various specific embodiments, in vitro assays can be carried out with a patient's T cells, to determine if a Therapeutic has a desired effect upon such cells.

In another embodiment, T cells capable of being activated are plated out or grown in vitro, and exposed to a Therapeutic. The Therapeutic which results in a cell phenotype that is more normal (i.e., less representative of a pre-neoplastic state, neoplastic state, malignant state, or transformed phenotype) is selected for therapeutic use. Many assays standard in the art can be used to assess whether a pre-neoplastic state, neoplastic state, or a transformed or malignant phenotype, is present. For example, characteristics associated with a transformed phenotype (a set of in vitro characteristics associated with a tumorigenic ability in vivo) include a more rounded cell morphology, looser substratum attachment, loss of contact inhibition, loss of anchorage dependence, release of proteases such as plasminogen activator, increased sugar transport, decreased serum requirement, expression of fetal antigens, disappearance of the 250,000 dalton surface protein, etc. (see Luria et al., GENERAL VIROLOGY, 3D ED., JOHN WILEY & SONS, New York pp. 436-446 (1978)).

In other specific embodiments, the in vitro assays described supra can be carried out using a cell line, rather than a cell sample derived from the specific patient to be treated, in which the cell line is derived from or displays characteristic(s) associated with the malignant, neoplastic or pre-neoplastic disorder desired to be treated or prevented, or is derived from the cell type upon which an effect is desired, according to the present invention.

Compounds for use in therapy can be tested in suitable animal model systems prior to testing in humans, including but not limited to rats, mice, chicken, cows, monkeys, rabbits, etc. For in vivo testing, prior to administration to humans, any animal model system known in the art may be used.

### 5.7.3.3. THERAPEUTIC/PROPHYLACTIC ADMINISTRATION AND COMPOSITIONS

The invention provides methods of treatment (and prophylaxis) by administration to a subject of an effective amount of a Therapeutic of the invention. In a preferred aspect, the Therapeutic is substantially purified. The subject is preferably an animal, including but not limited to animals such as cows, pigs, horses, chickens, cats, dogs, etc., and is preferably a mammal, and most preferably human. In a specific embodiment, a non-human mammal is the subject.

Formulations and methods of administration that can be employed when the Therapeutic comprises a nucleic acid are described in Section 5.7.1 above; additional appropriate formulations and routes of administration can be selected from among those described herein below.

Various delivery systems are known and can be used to administer a Therapeutic of the invention, e.g., encapsulation in liposomes, microparticles, microcapsules, recombinant cells capable of expressing the Therapeutic, receptor-mediated endocytosis (see, e.g., Wu and Wu, J. Biol. Chem. 262:4429-4432 (1987)), construction of a Therapeutic nucleic acid as part of a retroviral or other vector, etc. Methods of introduction include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

In another embodiment, the Therapeutic can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the Therapeutic can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Pewas I J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the thymus, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment where the Therapeutic is a nucleic acid encoding a protein Therapeutic, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, DuPont), or coating with lipids or cell-surface receptors or transfecting agents, or by administering it in linkage to a homeobox-like peptide which is known to enter the nucleus (see e.g., Joliot et al., Proc. Natl. Acad. Sci. U.S.A. 88:1864-1868 (1991)), etc. Alternatively, a nucleic acid Therapeutic can be introduced intracellularly and incorporated within host cell DNA for expression, by homologous recombination.

The present invention also provides pharmaceutical compositions. Such compositions comprise a therapeutically effective amount of a Therapeutic, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the Therapeutic, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The Therapeutics of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the Therapeutic of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10 % to 95% active ingredient.

The invention also provides a pharmaceutical pack or kit comprising one or more containers filled with one or more of the ingredients of the pharmaceutical compositions of the invention. In one embodiment, the kit provides a container having a therapeutically-active amount of a TCAP. Optionally associated with such container(s) can be a notice in the form prescribed by a governmental agency regulating the manufacture, use or sale of pharmaceuticals or biological products, which notice reflects approval by the agency of manufacture, use or sale for human administration.

### 5.7.4. SCREENING FOR TCAP AGONISTS AND ANTAGONISTS

TCAP nucleic acids, proteins, and derivatives also have uses in screening assays to detect molecules that specifically bind to TCAP nucleic acids, proteins, or derivatives and thus have potential use as agonists or antagonists of TCAP, in particular, molecules that thus affect T cell activation and/or proliferation. In a preferred embodiment, such assays are performed to screen for molecules with potential utility as anti-cancer drugs or lead compounds for drug development. The invention thus provides assays to detect molecules that specifically bind to TCAP nucleic acids, proteins, or derivatives. For example, recombinant cells expressing TCAP nucleic acids can be used to recombinantly produce TCAPs in these assays, to screen for molecules that bind to a TCAP. Molecules (e.g., putative binding partners of TCAP) are contacted with a particular TCAP or fragment thereof under conditions conducive to binding, and then molecules that specifically bind to the TCAP are identified. Similar methods can be used to screen for molecules that bind to TCAP derivatives or nucleic acids. Methods that can be used to carry out the foregoing are commonly known in the art.

By way of example, diversity libraries, such as random or combinatorial peptide or nonpeptide libraries can be screened for molecules that specifically bind to a particular TCAP. Many libraries are known in the art that can be used, e.g., chemically synthesized libraries, recombinant (e.g., phage display libraries), and in vitro translation-based libraries.

Examples of chemically synthesized libraries are described in Fodor et al., Science 251:767-773 (1991); Houghten et al., Nature 354:84-86 (1991); Lam et al., Nature 354:82-84 (1991); Medynski, Bio/Technology 12:709-710 (1994); Gallop et al., J. Medicinal Chemistry 37(9):1233-1251 (1994); Ohlmeyer et al., Proc. Natl. Acad. Sci. U.S.A. 90:10922-10926 (1993); Erb et al., Proc. Natl. Acad. Sci. U.S.A. 91:11422-11426 (1994); Houghten et al., Biotechniques 13:412 (1992); Jayawickreme et al., Proc. Natl. Acad. Sci. U.S.A. 91:1614-1618 (1994); Salmon et al., Proc. Natl. Acad. Sci. U.S.A. 90:11708-11712 (1993); PCT Publication No. WO 93/20242; and Brenner and Lerner, Proc. Natl. Acad. Sci. U.S.A. 89:5381-5383 (1992).

Examples of phage display libraries are described in Scott and Smith, Science 249:386-390 (1990); Devlin et al., Science, 249:404-406 (1990); Christian, R. B., et al., J. Mol. Biol. 227:711-718 (1992)); Lenstra, J. Immunol. Meth. 152:149-157 (1992); Kay et al., Gene 128:59-65 (1993); and PCT Publication No. WO 94/18318 published Aug. 18, 1994.

In vitro translation-based libraries include but are not limited to those described in PCT Publication No. WO 91/05058 published Apr.18, 1991; and Mattheakis et al., Proc. Natl. Acad. Sci. U.S.A. 91:9022-9026 (1994).

By way of examples of nonpeptide libraries, a benzodiazepine library (see e.g., Bunin et al., Proc. Natl. Acad. Sci. U.S.A. 91:4708-4712 (1994)) can be adapted for use. Peptoid libraries (Simon et al., Proc. Natl. Acad. Sci. U.S.A. 89:9367-9371 (1992)) can also be used. Another example of a library that can be used, in which the amide functionalities in peptides have been permethylated to generate a chemically transformed combinatorial library, is described by Ostresh et al. (Proc. Natl. Acad. Sci. U.S.A. 91:11138-11142 (1994)).

Screening the libraries can be accomplished by any of a variety of commonly known methods. See, e.g., the following references, which disclose screening of peptide libraries: Parmley and Smith, Adv. Exp. Med. Biol. 251:215-218 (1989); Scott and Smith, Science 249:386-390 (1990); Fowlkes et al., BioTechniques 13:422-427 (1992); Oldenburg et al., Proc. Natl. Acad. Sci. U.S.A. 89:5393-5397 (1992); Yu et al., Cell 76:933-945 (1994); Staudt et al., Science 241:577-580 (1988); Bock et al., Nature 355:564-566 (1992); Tuerk et al., Proc. Natl. Acad. Sci. U.S.A. 89:6988-6992 (1992); Ellington et al., Nature 355:850-852 (1992); U.S. Pat. No. 5,096,815, U.S. Pat. No. 5,223,409, and U.S. Pat. No. 5,198,346, all to Ladner et al.; Rebar and Pabo, Science 263:671-673 (1993); and PCT Publication No. WO 94/18318, published Aug. 8, 1994.

In a specific embodiment, screening can be carried out by contacting the library members with a TCAP protein (or nucleic acid or derivative) immobilized on a solid phase and harvesting those library members that bind to the protein (or nucleic acid or derivative). Examples of such screening methods, termed “panning” techniques are described by way of example in Parmley and Smith, Gene 73:305-318 (1988); Fowlkes et al., BioTechniques 13:422-427 (1992); PCT Publication No. WO 94/18318; and in references cited herein above.

In another embodiment, the two-hybrid system for selecting interacting proteins in yeast (Fields and Song, Nature 340:245-246 (1989); Chien et al., Proc. Natl. Acad. Sci. U.S.A. 88:9578-9582 (1991)) can be used to identify molecules that specifically bind to a TCAP protein or derivative.

In another embodiment, screening can be carried out by creating a peptide library in a prokaryotic or eukaryotic cells, such that the library proteins are expressed on the cells' surface, followed by contacting the cell surface with a TCAP and determining whether binding has taken place. Alternatively, the cells are transformed with a nucleic acid encoding a TCAP, such that the TCAP is expressed on the cells' surface. The cells are then contacted with a potential agonist or antagonist, and binding, or lack thereof, is determined. In a specific embodiment of the foregoing, the potential agonist or antagonist is expressed in the same or a different cell such that the potential agonist or antagonist is expressed on the cells' surface.

### 5.7.5. TRANSGENIC ANIMALS

The invention also provides animal models. Transgenic animals that have incorporated and express a constitutively-functional TCAP gene have use as animal models of diseases and disorders involving in T cell overactivation or over-proliferation, or in which cell proliferation is desired. Such animals can be used to screen for or test molecules for the ability to suppress activation and/or proliferation of T cells and thus treat or prevent such diseases and disorders. In one embodiment, animal models for diseases and disorders involving T cell activation (e.g., as described in Section 5.7.5) are provided. Such animals can be initially produced by promoting homologous recombination between a TCAP gene in its chromosome and an exogenous TCAP gene that has been rendered biologically inactive. Preferably the sequence inserted is a heterologous sequence, e.g., an antibiotic resistance gene. In a preferred aspect, this homologous recombination is carried out by transforming embryo-derived stem (ES) cells with a vector containing an insertionally inactivated gene, wherein the active gene encodes a particular TCAP, such that homologous recombination occurs; the ES cells are then injected into a blastocyst, and the blastocyst is implanted into a foster mother, followed by the birth of the chimeric animal, also called a “knockout animal,” in which a TCAP gene has been inactivated (see Capecchi, Science 244:1288-1292 (1989)). The chimeric animal can be bred to produce additional knockout animals. Chimeric animals can be and are preferably non-human mammals such as mice, hamsters, sheep, pigs, cattle, etc. In a specific embodiment, a knockout mouse is produced.

Such knockout animals are expected to develop or be predisposed to developing diseases or disorders involving T cell underproliferation and thus can have use as animal models of such diseases and disorders, e.g., to screen for or test molecules for the ability to promote activation or proliferation and thus treat or prevent such diseases or disorders.

In a different embodiment of the invention, transgenic animals that have incorporated and express a constitutively-functional TCAP gene have use as animal models of diseases and disorders involving in T cell overactivation, or in which T cell activation is desired. Such animals can be used to screen for or test molecules for the ability to suppress activation of T cells and thus treat or prevent such diseases and disorders.

In particular, each transgenic line expressing a particular key gene under the control of the regulatory sequences of a characterizing gene is created by the introduction, for example by pronuclear injection, of a vector containing the transgene into a founder animal, such that the transgene is transmitted to offspring in the line. The transgene preferably randomly integrates into the genome of the founder but in specific embodiments may be introduced by directed homologous recombination. In a preferred embodiment, the transgene is present at a location on the chromosome other than the site of the endogenous characterizing gene. In a preferred embodiment, homologous recombination in bacteria is used for target-directed insertion of the key gene sequence into the genomic DNA for all or a portion of the characterizing gene, including sufficient characterizing gene regulatory sequences to promote expression of the characterizing gene in its endogenous expression pattern. In a preferred embodiment, the characterizing gene sequences are on a bacterial artificial chromosome (BAC). In specific embodiments, the key gene coding sequences are inserted as a 5′ fusion with the characterizing gene coding sequence such that the key gene coding sequences are inserted in frame and directly 3′ from the initiation codon for the characterizing gene coding sequences. In another embodiment, the key gene coding sequences are inserted into the 3′ untranslated region (UTR) of the characterizing gene and, preferably, have their own internal ribosome entry sequence (IRES).

The vector (preferably a BAC) comprising the key gene coding sequences and characterizing gene sequences is then introduced into the genome of a potential founder animal to generate a line of transgenic animals. Potential founder animals can be screened for the selective expression of the key gene sequence in the population of cells characterized by expression of the endogenous characterizing gene. Transgenic animals that exhibit appropriate expression (e.g., detectable expression of the key gene product having the same expression pattern within the animal as the endogenous characterizing gene) are selected as founders for a line of transgenic animals.

Knockouts, including tissue-specific knockouts (in which the gene of interest is inactivated in particular tissues), can also be made by methods known in the art.

Accordingly, the invention provides a transgenic animal that comprises a recombinant non-human animal in which a gene encoding a protein comprising SEQ ID NO: 3, SEQ ID NO: 4, SEQ ID NO: 7, SEQ ID NO: 9, SEQ ID NO: 11, SEQ ID NO: 13, SEQ ID NO: 15, SEQ ID NO: 17, or SEQ ID NO: 19 has been inactivated by a method comprising introducing a nucleic acid into the plant or animal or an ancestor thereof, which nucleic acid or a portion thereof becomes inserted into or replaces said gene, or a progeny of such animal in which said gene has been inactivated.

### 6. EXAMPLES

The following examples are by way of illustration of the previously described invention, and are not limiting of that description in any way. In particular, the Examples presented herein below describe the analysis of the human T cell Activation GTPase Activating Protein and T cell Activation G-Protein coupled Receptor.

### Example 1

### Identification of Genes Upregulated During T Cell Activation

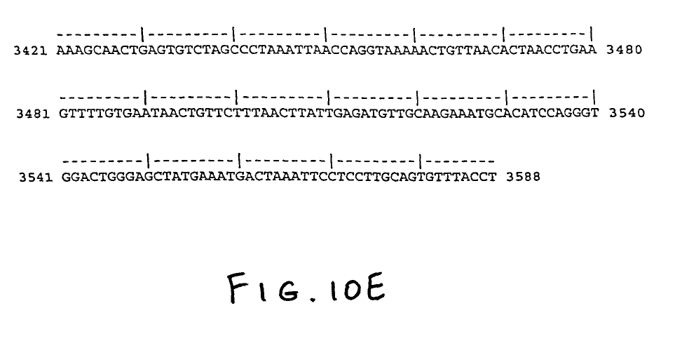

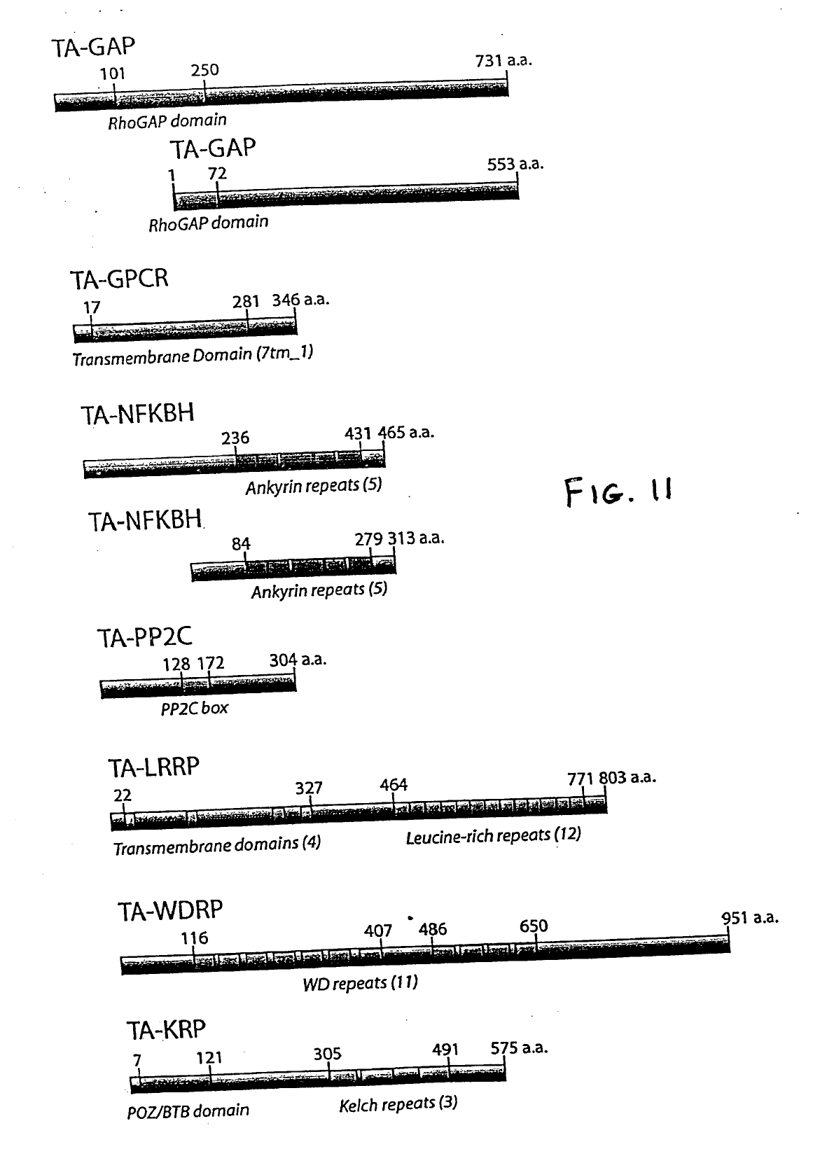

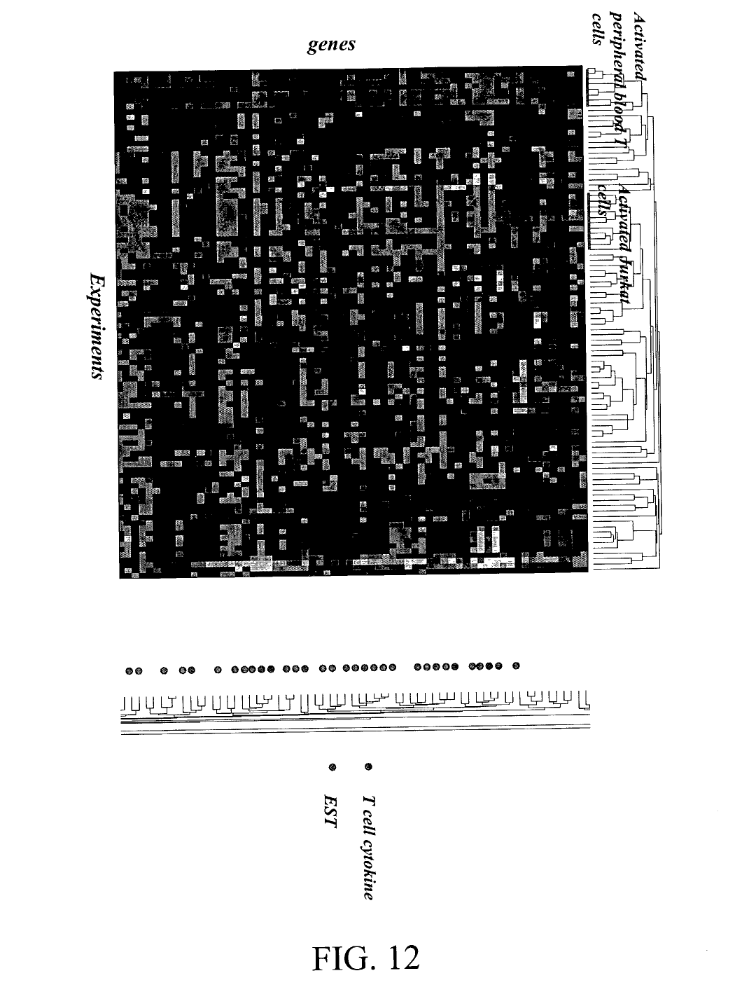

To identify genes upregulated during T cell activation, FlexJet™ chips representing either 25,000 or 50,000 Unigene clusters were hybridized to a mixture of cRNAs untreated versus treated cells of various types. FIG. 12 depicts a series of experiments comparing activated and unactivated Jurkat cells, K562 cells, peripheral blood T cells, THP1 cells, NB4 cells, JCAM cells, HL60 cells, and B-lymphoblast cells. A total of 3853 genes regulated >3-fold, P<0.01 in a total of 104 experiments were analyzed by a two dimensional hierarchical clustering algorithm. This analysis groups genes showing the greatest similarity of regulation over all experiments (first dimension) and the experiments showing the greatest similarities in gene regulation (second dimension). For clarity, FIG. 12 depicts only a section of the total data set (64 genes and 94 experiments). Each experiment and each gene are represented on the X and Y axes, respectively. Experiments involving activated peripheral blood T cells and activated Jurkat T cells are indicated with horizontal black bars. Genes upregulated in a particular experiment are colored dark gray; genes down regulated in that experiment are colored light gray; and genes showing no regulation in a particular experiment are colored black. The set of genes shown here demonstrates enrichment for T cell cytokines. Of the 3853 genes clustered, 24 (0.6%) encoded known cytokines. In the region shown, which comprises 64 genes, 9 (14%) were cytokines. Thus, there was a 27-fold enrichment for cytokine genes in this group. Known cytokine genes are highlighted with dark gray circles. This region also contains 21 ESTs of unknown function, which are indicated with black gray.

35 EST clusters were identified which clustered among T cell cytokines. When extended, these were found to represent 25 different transcripts. A total of 24 ESTs linked to known genes were identified (Table 1). Four of these 24 ESTs were found to map to introns of known genes. Ten of these 24 ESTs were found to overlap with cDNA sequences published during the course of this work. Fifteen of these ESTs were found to map in close proximity to the 3′ untranslated region (3′ UTR) of known genes, and have been tentatively identified as extensions of these 3′ UTRs. Three of these tentative identifications were confirmed by RT-PCR or genomic tiling (Bach2, TNFRSF9, IL2RA).

The remainder identified seven novel transcripts encoding a new GPCR, three new potential signal transducers (a phosphatase, a GTPase activating protein, and a WD-repeat containing protein); a potential NF-κB-like transcription factor, a keich motif-containing protein, and a leucine repeat-rich protein. These are discussed in more detail in Examples 3-9.

TABLE 1
Summary of ESTs identified as known genes by expression coregulation.

| | | | | Unigene ID | |
|---|---|---|---|---|---|
| | Likely gene | EST relationship | Known T-Cell | (Build #128, | |
| ESTs | identity | to gene | activation gene | Dec. 22, 2000) | gDNA or new cDNA |
| AA284303 | TNFSF8 | 3′ UTR of known gene | yes | Hs.101370 | AL133412, NM_001244 |
| AI308959 | IL21R | 3′ UTR of known gene | yes | Hs.126232 | AC002303 |
| AI418535 | IL2RA | 3′ UTR of known gene | yes | Hs.130058 | AL137186, NM_000417 |
| AA211393 | TNFRSF9 | 3′ UTR of known gene | yes | Hs.86447 | AL009183, NM_001561 |
| AI624755 | TNFRSF9 | 3′ UTR of known gene | yes | Hs.193418 | AL009183, NM_001561 |
| N63938 | Bach2 | 3′ UTR of known gene | no | Hs.88414 | AL353692 |
| AA825702 | Bach2 | 3′ UTR of known gene | no | Hs.88414 | AL353692 |
| AA488974 | Bach2 | 3′ UTR of known gene | no | Hs.88414 | AL353692 |
| AA251113 | Bach2 | 3′ UTR of known gene | no | Hs.88414 | AL353692 |
| AI655183 | REL | 3′ UTR of known gene | yes | Hs.105251 | AC010733, NM_002908 |
| AI652899 | REL | 3′ UTR of known gene | yes | Hs.86671 | AC010733, NM_002908 |
| AA210906 | REL | 3′ UTR of known gene | yes | Hs.188751 | AC010733, NM_002908 |
| AI497657 | GNG4 | 3′ UTR of known gene | no | Hs.135184 | AL162611, NM_004485 |
| AI608902 | B7-H1 | 3′ UTR of known gene | yes | Hs.106149 | NM_014143 |
| AI683598 | HSP105B | 3′ UTR of known gene | no | Hs.201615 | AL137142, NM_006644 |
| AI439019 | TBX21 | Identical to new cDNA | yes | Hs.272409 | NM_013351 |
| U19261 | TRAF1 | Identical to new cDNA | yes | Hs.2134 | NM_005658 |
| AI201323 | G18 | Identical to new cDNA | no | Hs.8257 | NM_013324 |
| AI377661 | PLSCR2 | Identical to new cDNA | no | Hs.123411 | NM_020359 |
| AI148659 | Fibronectin 1 | Identical to new cDNA | yes | Hs.287820 | AC026342, NM002153 |
| AI073984 | ICSPB1 | Identical to new cDNA | yes | Hs.14453 | NM_002163 |
| AI681868 | PBEF | Intron of known gene | yes | Hs.178784 | AC007032 |
| AI092511 | CD26 | Intron of known gene | yes | Hs.134533 | AC008063 |
| AA708350 | CDK6 | Intron of known gene | yes | Hs.189016 | AC000065, NM_001259 |

### Example 2

### Linkage of Exons Into Unigene Clusters by Array Expression Profiling

For details of the array-based techniques of exon clustering, mapping and extension using ESTs, see U.S. pat. app. No. 09/781,814.

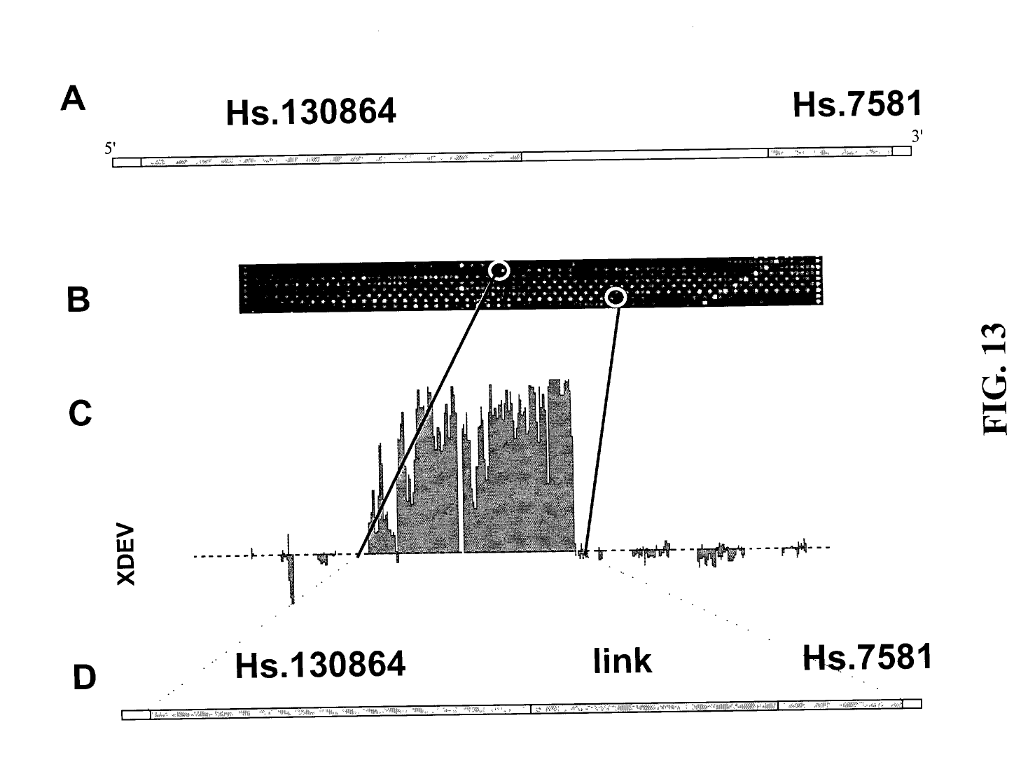

FIG. 13 depicts the use of array data to assign different sequences to the same transcript. Consensus sequences from two previously unlinked Unigene clusters, Hs. 7581 and Hs. 130864 (FIG. 13A) were mapped to a portion of human chromosome 6 as follows. FlexJetTM scanning arrays were synthesized specifying alternating sense and antisense oligonucleotides from every tenth nucleotide position in a genomic region encoding Unigene EST clusters, Hs. 7581 and Hs. 130864 on chromosome 6. Repetitive sequences in the genomic sequence were masked with the software program “RepeatMasker”. Nested 60 mer oligonucleotides were selected from every tenth position of both strands of non-repetitive sequence. FIG. 13B shows the array hybridized with a mixture of cRNA from activated (labeled with red fluorescent dye) and unactivated (labeled with green dye) Jurkat cells. Cells were activated by incubation for 4 hrs at 37° C. on plastic culture flasks coated with anti-TCR Vbeta8 monoclonal antibody (mAb) (Pharmingen), in the presence of PMA (10 nM) and soluble anti-CD28 (mAb) 9.3 μg/ml. Array data (FIG. 13B) showed contiguous hybridization, suggesting that this region, and therefore Hs. 7581 and Hs. 130864, hybridize with a single transcript.

The correlation between Hs. 7581 and Hs. 130864 was determined by XDEV measurements of hybridization over the region of chromosome 6 adjacent to Unigene clusters (FIG. 13C) Hs. 7581 and Hs. 130864. XDEV is a statistic defining the significance of a hybridization ratio in a two-color experiment:

X=(a₂−a₁)/[ρ₁²+ρ₂²+f²(a₁²+a₂²)]^{½}

where a_{1,2} are the intensities measured in the two channels for each spot, ρ_{1,2} are the uncertainties due to background subtraction, and f is a fractional multiplicative error such as would come from hybridization non-uniformities, fluctuations in the dye incorporation efficiency, scanner gain fluctuations, etc. Higher XDEV measurements represent more significant hybridization ratios. The region in FIG. 13B between the white circles corresponds to the peak of XDEV measurements.

The linkage of these EST clusters was confirmed by RT-PCR analysis. Further extension of these EST clusters by RT-PCR analysis revealed that this genomic region represents an exon from the 3′ untranslated region of the human homolog of the transcription factor, Bach2.

### Example 3

### Identification of TA-GAP

The cloning of the gene encoding human T cell activation-associated GTPase activating protein (TA-GAP), and analysis of the protein, was accomplished as follows.

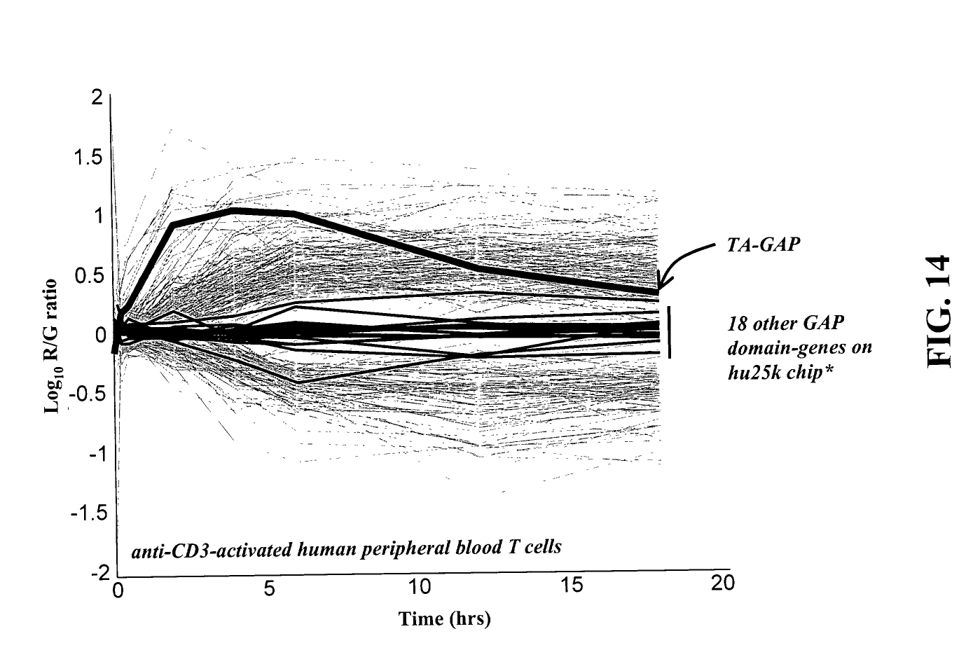

Human peripheral blood mononuclear cells were activated for 5 days with phytohemaglutinin (PHA), rested for one day in medium lacking PHA, and restimulated for the various periods of time on anti-CD3 (Pharmingen) coated plastic wells. At the indicated times, cells were harvested, cellular RNA was prepared, and amplified into cRNA. Hybridizations to human 25 k gene chips were performed with a mixture of cRNA from activated cells (red dye) and unactivated cells (green dye). FIG. 14 shows the time course of genes upregulated or downregulated during T cell activation. Transcripts showing significant regulation (>2-fold change and P<0.0001 in most samples) are shaded light gray. Transcripts encoding GAP-domain-containing proteins are depicted as dark gray lines. The TA-GAP transcript is depicted by the thick dark gray line, and transcripts for 18 other GAP domain-containing proteins (KIAA1501, KIAA0660, AI479025, ABR, GIT1, GIT2, ARHGAP1, ARHGAP4, G38P, GAPCENA, GAPL, IQGAP1, IQGAP2, NGAP, RAB3GAP, RANGAP1, RAP1GA1, RASA1) are depicted by thin dark gray lines. Of the transcripts tested that encode GAP-domain containing proteins, TA-GAP is the only one to show significant upregulation upon T cell activation.

TA-GAP was identified by investigation of a transcript corresponding to an EST, AI253155. AI253155 was found to be coregulated with T cell cytokine transcripts, and was homologous to a genomic clone, AL035530, on chromosome 6q25.3-27. An ENSEMBL predicted transcript, ENST00000037330, mapped 5′ to EST AI253155. The predicted transcript encoded a protein having homology to a GTPase-activator protein domain. cDNA corresponding to actual transcripts was amplified by RT-PCR using RNA from activated Jurkat cells as template, cloned and subjected to DNA sequence analysis.

Two cDNA sequences were identified (Table 2). The nucleotide sequences of the cDNAs were used to query the GenBank sequence database operated by the National Library of Medicine, in a BLAST (Basic Local Alignment Search Tool) search. A BLAST search returns an Expect (E) value; the E value is the probability that a particular search result would have occurred by chance. Highly significant E values are greatly smaller than 1.0 (but larger than 0.0), while insignificant E values are close to 1.0. Similarity of protein sequences was calculated after the manner of BLAST 2.0. Specifically, Amino acids paired by sequence alignment were compared using the BLOSUM62 scoring matrix (for a methods review, see: W R Pearson. Effective protein sequence comparison. Methods Enzymol 1996;266:227-58). BLOSUM62 is a rectangular matrix of values placed on each pair of aligned amino acids. The amino-acid pair values are designed to reflect the likelihood of amino acid replacement in conserved proteins. Positive scores are given to identities and conservative substitutions. Zero or negative scores are given for nonconservative substitutions.

For the purposes of generating these numbers, the column corresponding to each patent-sequence amino acid was found in the BLOSUM62 matrix. The appropriate row of BLOSUM62 was found for each aligned amino acid in the target sequence. The score at the intersection of the row and column was examined. If the number was positive, the amino acids were determined to be similar. If it was negative, the amino acids were determined not to be similar. Similarites were summed across alignments in the same manner as identities were summed. Amino acid sequences of the predicted protein products were compared to entries in two protein motif databases, Pfam and PROSITE. A Pfam score close to 0.0 indicates that the match(es) returned is highly significant.

The first cDNA sequence (Table 2: SEQ ID NO: 1) contained a full open reading frame that encoded a protein identical to the predicted protein from the ENSEMBL predicted transcript, but contained an additional 105 amino acids at the amino terminus (Table 3, SEQ ID NO: 3). The second cDNA sequence was a putative splice variant (Table 2, SEQ ID NO: 2), which contained a full open reading frame, but which encoded a smaller protein identical to the ENSEMBL predicted protein (Table 3, SEQ ID NO: 4). SEQ. ID NO: 1 aligned with its putative translation product SEQ ID NO: 2, and SEQ ID NO: 3 aligned with its putative translation product SEQ ID NO: 4, are depicted in FIGS. 1A-1E and 2A-2D, respectively. Analysis of the TA-GAP transcript during T cell activation revealed that it was transiently expressed and reached maximal levels after approximately four hours of activation (FIG. 14). There were 18 other GAP domain genes represented on the human 25 k chip used in these experiments, and TA-GAP was more highly regulated than any of these (FIG. 13).

TABLE 2
BLAST results for two TA-GAP-encoding cDNA sequences.

| Novel cDNA | Polypeptide | Novel cDNA Blast | Novel cDNA Blast | 125 bp % | 275 bp % | 100% |
|---|---|---|---|---|---|---|
| SEQ ID NO | SEQ ID NO | Score | Description | Identity | Identity | Identity Length |
| 1 | 3 | 3947 E = 0 | AL035530.1 Human | 100%. | 100% | 1991 |
| | | | DNA | | | |
| | | | sequence from clone RPI | | | |
| | | | (genomic BAC clone) | | | |
| | | 393 E = 1e−106 (exon 7) | | 100% | 72% | 198 |
| | | 294 E = 2e−55 (exon 8) | AK025272 | | | |
| | | | Homo sapiens | | | |
| | | | FLJ21619 fis | | | |
| 2 | 4 | 3947 E = 0 | AL0355350.1 Human | 100% | 100% | 1991 |
| | | | DNA | | | |
| | | | sequence from clone RPI | | | |
| | | | (genomic BAC clone) | | | |
| | | 393 E = 1e−106(exon 7) | | 100% | 72% | 198 |
| | | 224 E = 2e−55 (exon 8) | AK025272 | | | |
| | | | Homo sapiens cDNA: | | | |
| | | | FLJ21619 fis | | | |

TABLE 3
Protein database search results for two TA-GAP variants.

| | | | | Prosite | 100% Identity | 100% Similarity |
|---|---|---|---|---|---|---|
| SEQ ID NO | Blast Score | Blast Description | Pfam Motif(s) | Motif(s) | Length | Length |
| 3 | 1. 161, E = 3e−38 | 1. BAA92629.1 | RhoGAP domain | None | 5 | 10 |
| | | (AB037812) | (from residue 101 to | remarkable | | |
| | | KIAA1391 protein | residue 250) score = | | | |
| | | [Homo sapiens] | 101,3, E = 8.1e−28 | | | |
| | 2. 93, E = 1e−17 | 2. A49678 GTPase- | | | 7 | 11 |
| | | activating protein | | | | |
| | | RhoGAP | | | | |
| 4 | 1. 65, E = 23-09 | 1. BAA92629.1 | RhoGAP domain | None | 4 | 9 |
| | | (AB037812) | (from residue 6 to | remarkable | | |
| | | KIAA1391 protein | residue 96) score = | | | |
| | | [Homo sapiens] | −59.9, E = 0.31 | | | |
| | 2. 45.1, E = 2e−03 | 2. NP_061830 | | | 3 | 8 |
| | | SH3- domain | | | | |
| | | binding protein 1) | | | | |

### Example 4

### Identification of TA-GPCR

The cloning of the gene encoding human T cell activation associated G protein-coupled receptor (TA-GPCR), and analysis of the protein, was accomplished as follows.

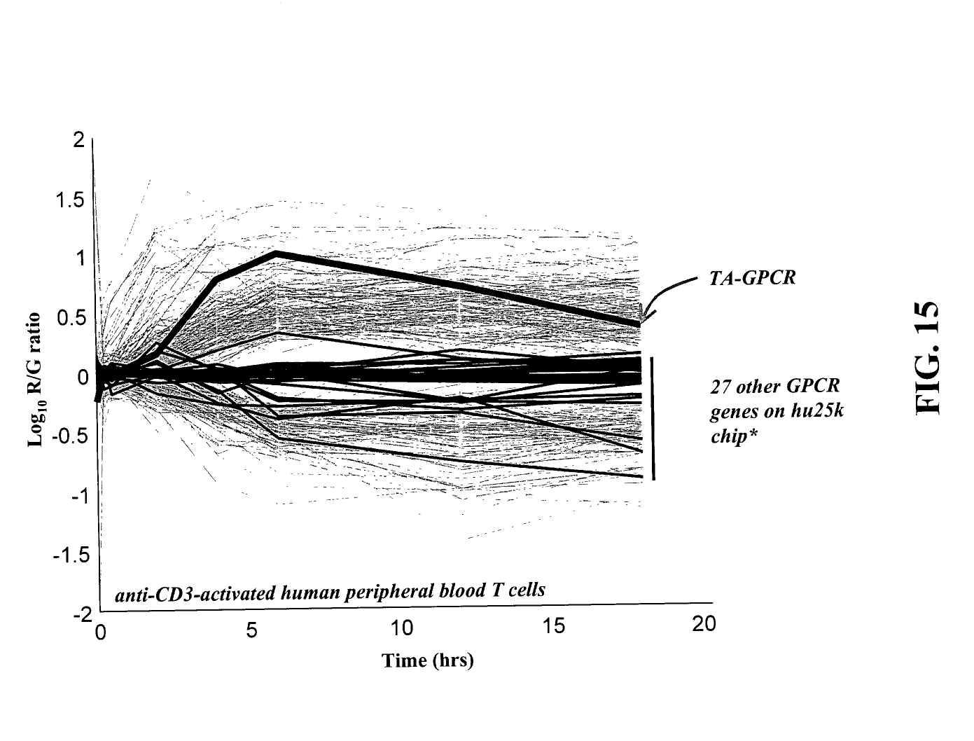

Analysis of the TA-GPCR transcript during T cell activation revealed that it reached maximal levels after approximately six hours of activation (FIG. 15). 27 other GPCR genes were represented on the human 25 k chip used in these experiments, and TA-GPCR was more highly regulated than any of these. Transcripts showing significant regulation (>2-fold change and P<0.0001 in most samples) in the experiment shown in FIG. 15 are depicted as thin gray lines. Transcripts encoding GPR proteins are colored red. The TA-GPCR transcript is depicted by the thick dark gray line, and transcripts for 27 other GPCR proteins are depicted by thin dark gray lines (GPR39, GPR51, AI61367, AI208357, GPRK6, GPRK5, GPR51, GPR19, AI659657, GPR48, EBI2, GPRK5, GPRK6, GPR68, GPR4, GPR9, LANCL1, CCR1, CCR4, CCR5, CCR7, CCR8, CMKLR1, CXCR4, HM74, LTBR4, AA040696). Of the transcripts tested that encode GPCRs, the ones encoding TA-GPCR were the only ones to show significant upregulation.

TA-GPCR was identified by investigation of a transcript corresponding to an EST, AA040696. AA040696 was coregulated with T cell cytokine transcripts, and was homologous to a genomic clone, AC026331, on chromosome 12. An ENSEMBL predicted transcript, AC026331.00004.443292, mapped 5′ to EST AA040696. The predicted transcript encoded a protein having homology to a novel GPR. cDNAs corresponding to actual transcript(s) were amplified by RT-PCR from RNA isolated from activated Jurkat cells as template, cloned and subjected to DNA sequence analysis. Two cDNA sequences (SEQ ID NOS: 5, 6) were identified, in roughly equivalent amounts. Both contained a full open reading frame, and both encoded a protein (SEQ ID NO: 7) identical to the predicted protein from the ENSEMBL predicted transcript. Alignment of the predicted ORF of SEQ ID NOS: 5 and 6 with the putative translation product SEQ ID NO: 7 are shown in FIGS. 3A-3E and 4A-4C, respectively. Nucleic acid and amino acid sequence comparisons, performed as described in Example 3, revealed that the cDNAs and predicted protein product had high sequence homology to G protein-coupled receptors (Tables 4, 5). Based on BLAST search results, TA-GPCR is a Class A GPCR.

TABLE 4
BLAST search results for two TA-GPCR-encoding cDNA sequences.

| Novel cDNA | Polypeptide | Novel cDNA Blast | Novel cDNA Blast | 125 bp % | 275 bp % | 100% |
|---|---|---|---|---|---|---|
| SEQ ID NO | SEQ ID NO | Score | Description | Identity | Identity | Identity Length |
| 5 | 7 | 357, E = 3e−95 | AL354720.14 Human | 93.6% | 90.6% | 49 |
| | | | DNA sequence from | | | |
| | | | clone RP11-5-5F3) | | | |
| | | 351 E = 3e−95 | AC005529.7 | 94.4% | 90.9% | 52 |
| | | | Homo sapiens | | | |
| | | | chromosome 22q12 clone | | | |
| 6 | 7 | 349, E = 4e−93 | AL109923.29 Human | 94.4% | 91.3% | 34 |
| | | | DNA sequence from | | | |
| | | | clone RP3-46801 | | | |
| | | 345 E = 6e−92 | AC005912.1 | 92% | 90.9% | 29 |
| | | | Homo sapiens | | | |
| | | | chromosome | | | |
| | | | 12p13.3 BAC | | | |
| | | | RPCI11-543P15 | | | |

TABLE 5
Protein database search results for two TA-GPCR Variants.

| | | | | | 100% Identity | 100% Similarity |
|---|---|---|---|---|---|---|
| SEQ ID NO | Blast Score | Blast Description | Pfam Motif(s) | Prosite Motif(s) | Length | Length |
| 7 | 325 | NP_006009.1 | 7tm_1, 7 | Residues 107-123, | 14 | 25 |
| | E = 7e−88 | putative chemokine | transmembrane | PDOC00210 PS00237 | | |
| | | receptor (HM74) | receptor (rhodopsin | G_PROTEIN_{—} | | |
| | | | family) domain | RECEP_F1_{—} | | |
| | | | (residues 32-202), | 1G-protein | | |
| | | | score = 95.1, | coupled receptors | | |
| | | | E = 5e−21 | family 1 signature | | |
| | 320, E = 2e−86 | AJ300198 | | | | |
| | | Putative seven | | | | |
| | | transmembrane | | | | |
| | | spanning receptor | | | | |

TA-GPCR and the other indicated GPRs were subjected to multiple sequence alignment using BlockMaker (available on the Internet at blocks.fhcrc.org). This sequence comparison of the amino acid sequence of TA-GPCR with that of other G protein-coupled receptors revealed that TA-GPCR was more closely related to adenosine receptors than chemokine receptors.

### Example 5

### Identification of TA-PP2C

The cloning of a cDNA encoding human T cell activation associated serine-threonine class 2C phosphatase (TA-PP2C), and analysis of the encoded protein, was accomplished generally as described in Examples 3 and 4. A cDNA of 3748 nucleotides (SEQ ID NO: 8) was identified, which contained a full open reading frame predicted to encode a protein of 304 amino acids (SEQ ID NO: 9). An alignment of SEQ ID NO: 8 and its predicted product SEQ ID NO: 9 are shown in FIG. 5. Nucleic acid and amino acid sequence comparisons, performed as described in Example 3, revealed that the predicted protein product contained a sequence at amino acid residues 128-172 homologous to a protein phosphatase class 2C domain (Tables 6, 7). TA-PP2C is predicted to be a serine-threonine class 2C phosphatase.

TABLE 6
BLAST search results for a TA-PP2C-encoding cDNA sequence.

| Novel cDNA | Polypeptide | Novel cDNA | Novel cDNA | 125 bp % | 275 bp % | 100% Identity |
|---|---|---|---|---|---|---|
| SEQ ID NO | SEQ ID NO | Blast Score | Blast Description | Identity | Identity | Length |
| 8 | 9 | 255, E = 0 | ACC002350 | 100% | 100% | 2666 |
| | | | Human chr 12q24 | | | |
| | | | PAC RPC13- | | | |
| | | | 424M6 | | | |

TABLE 7
Protein database search results for TA-PP2C.

| | | | | | | 100% |
|---|---|---|---|---|---|---|
| SEQ | | | | | 100% | Simi- |
| ID | Blast | Blast | Pfam | Prosite | Identity | larity |
| NO | Score | Description | Motif(s) | Motif(s) | Length | Length |
| 9 | 255, | AAF47506 | Protein | N/A | 13 | 22 |
| | E = 6e−67 | CG12091 | phospha- | | | |
| | | gene product | tase 2C | | | |
| | | (Drosophila) | domain, | | | |
| | | | residues | | | |
| | | | 128-172 | | | |

### Example 6

### Identification of TA-NFKIBH

The cloning of a cDNA encoding human T cell activation associated NF-κB-like transcription factor (TA-NFKBH), and analysis of the encoded protein, was accomplished generally as described in Examples 3 and 4. A cDNA of 1736 nucleotides (SEQ ID NO: 10) and a cDNA of 1834 nucleotides were identified (SEQ ID NO: 12), which contained a full open reading frames predicted to encode proteins of 465 amino acids (SEQ ID NO: 11) and 313 amino acids (SEQ ID NO: 13), respectively. The short variant has the same amino acid sequence as SEQ ID NO: 11, amino acids 153-465. An alignment of SEQ ID NO: 10 to SEQ ID NO: 11, and SEQ ID NO: 12 to SEQ ID NO: 13 are shown in FIGS. 6 and 7, respectively. Nucleic acid and amino acid sequence comparisons, performed as described in Example 3, revealed that both predicted protein products had Ank (ankyrin-like) repeats, which are involved in protein-protein interactions. The long form has Ank repeats at residues 200-439, particularly in 236-268, 269-301 and 395-431. Both forms show sequence homology to NF-κB or to MAIL, a murine κB transcriptional activator (Tables 8, 9). TA-NFKBH is predicted to be an NF-κB—like transcription factor.

TABLE 8
BLAST search results for two TA-NFKBH-encoding cDNA sequences.

| Novel cDNA | Polypeptide | Novel cDNA | Novel cDNA | 125 bp % | 275 bp % | 100% Identity |
|---|---|---|---|---|---|---|
| SEQ ID NO | SEQ ID NO | Blast Score | Blast Description | Identity | Identity | Length |
| 10 | 11 (long) | 739, E = 0 | AD000864 | 100% | 100% | 373 |
| | | | Human DNA | | | |
| | | | sequence from | | | |
| | | | chr. 19, cosmid | | | |
| | | | R28051 | | | |
| 12 | 13 (short) | 739, E = 0 | AD000864 | 100% | 100% | 373 |
| | | | Human DNA | | | |
| | | | sequence from | | | |
| | | | chr. 19, cosmid | | | |
| | | | R28051 | | | |

TABLE 9
Protein database search results for two TA-NFKBH variants.

| | | | | | 100% Identity | 100% Similarity |
|---|---|---|---|---|---|---|
| SEQ ID NO | Blast Score | Blast Description | Pfam Motif(s) | Prosite Motif(s) | Length | Length |
| 11 (long) | 179, E = 8e−44 | BAB18302 MAIL | 5 Ank repeats | Proline-rich region, | 11 | 12 |
| | | (Mus musculus) | | residues 70-177 | | |
| | 87, E = 4e−16 | NP_003989 | | Ank repeats, residues | 6 | 7 |
| | | NF-kappa B p105 | | 200-439, 236-268, | | |
| | | homolog | | 269-301, 395-431 | | |
| 13 (short) | 197, E = 2e−49628, | BAB18302 MAIL | 5 Ank repeats, | Ank repeats | 11 | 12 |
| | E = e−179 | (Mus musculus) | 84-279 | | | |
| | 100, E = 2e−20 | NP_005169.1 | | | 5 | 12 |
| | | B-cell | | | | |
| | | CLL/lymphoma | | | | |

### Example 7

### Identification of TA-WDRP

The cloning of a cDNA encoding human T cell activation associated transducin-like protein with WD motifs (TA-WDRP), and analysis of the encoded protein, was accomplished generally as described in Examples 3 and 4. A cDNA of 3049 nucleotides (SEQ ID NO: 14) was identified, which contained a full open reading frame predicted to encode a protein of 951 amino acids (SEQ ID NO: 15). An alignment of SEQ ID NO: 14 to SEQ ID NO: 15 is shown in FIG. 8. Nucleic acid and amino acid sequence comparisons, performed as described in Example 3, revealed that the cDNAs and predicted protein product had sequence homology to transducins, which are G-proteins (Tables 10, 11). TA-WDRP is also predicted to contain a WD motif repeats at amino acid residues 116-149, 180-216, 223-259, 362-398, 407-443, 449-484, and 490-526.

TABLE 10
BLAST search results for a TA-WDRP-encoding cDNA sequence.

| Novel cDNA | Polypeptide | Novel cDNA | Novel cDNA | 125 bp % | 275 bp % | 100% Identity |
|---|---|---|---|---|---|---|
| SEQ ID NO | SEQ ID NO | Blast Score | Blast Description | Identity | Identity | Length |
| 14 | 15 | 890, E = 0 | AC020925 Chr. 5 | 100% | 100% | 449 |
| | | | clone CTD- | | | |
| | | | 2134K2 | | | |

TABLE 11
Protein database search results for TA-WDRP.

| | | Blast | | | 100% Identity | 100% Similarity |
|---|---|---|---|---|---|---|
| SEQ ID NO | Blast Score | Description | Pfam Motif(s) | Prosite Motif(s) | Length | Length |
| 15 | 628, E = e−179 | AAF54941 | G-protein beta | AMP- | 8 | 19 |
| | | (AE003700) | WD-40 repeats | dependent | | |
| | | CG9799 | (PF0400) | synthetase | | |
| | | (Drosophila) | | and ligase | | |
| | | | | (PS00455) | | |
| | 494, E = e−138 | CAB81036 | | G-protein beta | 8 | 23 |
| | | (AL161502) | | WD-40 repeats | | |
| | | putative | | (S00167, | | |
| | | WD-repeat | | PS50082, | | |
| | | membrane | | PS50294) | | |
| | | protein | | | | |
| | | (Arabidopsis) | | | | |

### Example 8

### Identification of TA-KRP

The cloning of a cDNA encoding human T cell activation associated kelch-like transcription factor (TA-KRP), and analysis of the encoded protein, was accomplished generally as described in Examples 3 and 4. A cDNA of 4617 nucleotides (SEQ ID NO: 16) was identified, which contained a full open reading frame predicted to encode a protein of 575 amino acids (SEQ ID NO: 17). An alignment of SEQ ID NO: 16 to SEQ ID NO: 17 is shown in FIG. 9. Nucleic acid sequence comparisons, performed as described in Example 3, revealed that the predicted protein product contained a BPOZ/TB domain at residues 138-252, characteristic of a class of transcription regulatory proteins (Ahmad et al., Proc. Natl. Acad. Sci. U.S.A. 95:12123-12128 (1998)) (Tables 12, 13). The protein also contains four kelch repeats.

TABLE 12
BLAST search results for a TA-KRP-encoding cDNA sequence.

| Novel cDNA | Polypeptide | Novel cDNA | Novel cDNA | 125 bp % | 275 bp % | 100% Identity |
|---|---|---|---|---|---|---|
| SEQ ID NO | SEQ ID NO | Blast Score | Blast Description | Identity | Identity | Length |
| 16 | 17 | 4339, E = 0 | AC020655 | 100% | 100% | 3290 |
| | | | human BAC | | | |
| | | | RP11-15B4 | | | |

TABLE 13
Protein database search results for TA-KRP.

| | | | | | 100% Identity | 100% Similarity |
|---|---|---|---|---|---|---|
| SEQ ID NO | Blast Score | Blast Description | Pfam Motif(s) | Prosite Motif(s) | Length | Length |
| 17 | 221, E = 3e−56 | Kiaa1489 human | Kelch repeat | BTB/POZ domain | 10 | 13 |
| | | protein | (PF01344) | (PS50097) | | |
| | 187, E = 5e−46 | NP006054 | BTB/POZ domain | N/A | 5 | 9 |
| | | sarcomeric | (PF00651) | | | |
| | | muscle protein | | | | |

### Example 9

### Identification of TA-LRRP

The cloning of a cDNA encoding a human T cell activation associated leucine repeat-rich protein (TA-KRP), and analysis of the encoded protein, was accomplished generally as described in Examples 3 and 4. A cDNA of 3588 nucleotides (SEQ ID NO: 18) was identified, which contained a full open reading frame predicted to encode a protein of 803 amino acids (SEQ ID NO: 19). An alignment of SEQ ID NO: 18 to SEQ ID NO: 19 is shown in FIG. 10. Nucleic acid sequence comparisons, performed as described in Example 3, revealed that the predicted protein product contained 12 leucine-rich repeats, as well as a bipartite nuclear localization signal at residues 228-245 (Tables 14, 15).

TABLE 14
BLAST search results for a TA-LRRP-encoding cDNA sequence.

| | | | | | | 100% Identity |
|---|---|---|---|---|---|---|
| Novel cDNA | Polypeptide | Novel cDNA | Novel cDNA | 125 bp % | 275 bp % | Length |
| SEQ ID NO | SEQ ID NO | Blast Score | Blast Description | Identity | Identity | (nucleotides) |
| 18 | 19 | 3457, E = 0 | AD00864 | 100% | 100% | 1765 |
| | | | Human DNA | | | |
| | | | sequence from | | | |
| | | | chr. 19, | | | |
| | | | cosmid R28051 | | | |

TABLE 15
Protein database search results for TA-LRRP.

| SEQ | Blast | Blast | Pfam | Prosite | 100% Identity | 100% Similarity |
|---|---|---|---|---|---|---|
| ID NO | Score | Description | Motif(s) | Motif(s) | Length | Length |
| 19 | 850, | BAA92675 | 12 leucine | Bipartite nuclear | 16 | 38 |
| | E = 0 | (AB037858) | rich | localization signal | | |
| | | KIAA1437 | repeats | | | |
| | | (H. sapiens) | | | | |

### 7. REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

What is claimed is:

1. A purified protein comprising the amino acid sequence of SEQ ID NO: 17.

2. An isolated nucleic acid comprising the nucleotide sequence of SEQ ID NO: 16, or a coding region thereof, or the complement of any of the foregoing.

3. The isolated nucleic acid of claim 2 which is DNA.

4. An isolated nucleic acid comprising a nucleotide sequence encoding the protein of claim 1, or the complement thereof.

5. A recombinant cell containing the nucleic acid of claim 2, in which the nucleotide sequence is under the control of a promoter heterologous to the nucleotide sequence.

6. A recombinant cell containing a nucleic acid vector that comprises the nucleic acid of claim 2.

7. An antibody that binds to a protein consisting of the amino acid sequence of SEQ ID NO:17.

8. The antibody of claim 7 which is monoclonal.

9. A molecule comprising a fragment of the antibody of claim 7, which fragment binds a protein consisting of the amino acid sequence of SEQ ID NO: 17.

10. A method of producing a protein comprising:
growing a recombinant cell containing the nucleic acid of any one of claims 2-4 in which said nucleotide sequence is under the control of a promoter heterologous to said nucleotide sequence, such that the protein encoded by said nucleic acid is expressed by the cell; and
recovering said expressed protein.

11. An isolated protein that is the product of the process of claim 10.

12. A pharmaceutical composition comprising a therapeutically effective amount of the protein of claim 1, and a pharmaceutically acceptable carrier.

13. A pharmaceutical composition comprising a therapeutically effective amount of the nucleic acid of claim 2; and a pharmaceutically acceptable carrier.

14. A pharmaceutical composition comprising a therapeutically effective amount of the recombinant cell of claim 5 or claim 6; and a pharmaceutically acceptable carrier.

15. A pharmaceutical composition comprising a therapeutically effective amount of an antibody that binds to a protein comprising the amino acid sequence of claim 1, and a pharmaceutically acceptable carrier.

16. A method of measuring the level of T cell activation in a subject, comprising:
contacting a sample comprising mRNA or nucleic acid derived therefrom from a subject, with a nucleic acid probe that hybridizes to a nucleic acid that encodes the protein of claim 1 under conditions conducive to hybridization; and
measuring the amount of said probe that hybridizes to nucleic acid in the sample; wherein the amount of hybridization is indicative of the level of T cell activation.

17. A method of measuring the level of T cell activation in a subject, comprising:
contacting a sample derived from a patient with an antibody that binds the protein of claim 1, under conditions conducive to immunospecific binding; and
measuring the amount of any immunospecific binding by the antibody wherein the amount of said immunospecific binding is indicative of the level of T cell activation.


SEQUENCE LISTING (19 sequences)

SEQ ID NO: 1 - DNA, 3218 bp, Homo sapiens
gacatagctgccctaaaaggaatgaggaagcgagagctctccagtgtctggctggctccgtccgtgtgacagcccatgatgttctttccggtctctgtaatattctgaatttccacctgcccgccccttcgcttataatgcagagcatgtgaagggagaccggctcggtctctctctctcccagtggactagaaggagcagagagttatgctgtttctcccattctttacagctcaccggatgtaaaagaactctggctagagaccctccaaggacagaggcacagccacacgggagtgaaatccacccctggacagtcagccgcaatactgatgaagctgagaagcagccacaatgcttcaaaaacactaaacgccaataatatggagacactaatcgaatgtcaatcagagggtgatatcaaggaacatcccctgttggcatcatgtgagagtgaagacagtatttgccagctcattgaagttaagaagagaaagaaggtgctgtcctggccctttctcatgagaaggctctcccctgcatcagatttttctggggctttggagacagacttgaaagcatcgctatttgatcagcccttgtcaattatctgcggtgacagtgacacactccccagacccatccaggacattctcactattctatgccttaaaggcccttcaacggaagggatattcaggagagcagccaacgagaaagcccgtaaggagctgaaggaggagctcaactctggggatgcggtggatctggagaggctccccgtgcacctcctcgctgtggtctttaaggacttcctcagaagtatcccccggaagctactttcaagcgacctctttgaggagtggatgggtgctctggagatgcaggacgaggaggacagaatcgaggccctgaaacaggttgcagataagctcccccggcccaacctcctgctactcaagcacttggtctatgtgctgcacctcatcagcaagaactctgaggtgaacaggatggactccagcaatctggccatctgcattggacccaacatgctcaccctggagaatgaccagagcctgtcatttgaagcccagaaggacctgaacaacaaggtgaagacactggtggaattcctcattgataactgctttgaaatatttggggagaacattccagtgcattccagtatcacttctgatgactccctggagcacactgacagttcagatgtgtcgaccctgcagaatgactcagcctacgacagcaacgaccctgatgtggaatccaacagcagcagtggcatcagctctcccagcaggcagccccaggtgcccatggccacagctgctggcttggatagcgcgggcccacaggatgcccgagaggtcagcccagagcccattgtgagcaccgtggccaggctgaaaagctccctcgcacagcccgataggagatactcagagcccagcatgccatcctcccaggagtgcctcgagagccgggtgacaaaccaaacactaacaaagagtgaaggggacttccccgtgccccgggtaggctctcgtttggaaagtgaggaggctgaagacccatttccagaggaggtcttccctgcagtgcaaggcaaaaccaagaggccggtggacctgaagatcaagaacttggccccgggttcggtgctcccgcgggcactggttctcaaagccttctccagcagctcgctggacgcgtcctctgacagctcgcccgtggcttctccttccagtcccaaaagaaatttcttcagcagacatcagtctttcaccacaaagacagagaaaggcaagcccagccgagaaattaaaaagcactccatgtctttcacctttgcccctcacaaaaaagtgctgaccaaaaacctcagcgcgggctctgggaaatcgcaagactttaccagggaccacgtcccgaggggtgtcagaaaggaaagccagcttgccggccgaatcgtgcaggaaaatgggtgtgaaacccacaaccaaacagcccgcggcttctgcctgagaccccacgccctctcggtggatgatgtgttccagggagctgactgggagaggcctggaagcccaccctcttatgaagaggccatgcagggcccggcagccagactagtggcctccgagagccagaccgtggggagcatgacggtggggagcatgagggcgaggatgctggaggcgcactgcctcctaccccctcttccacctgctcaccacgtagaggactcaagacacaggggcagcaaagagccactccctggccacggactctctcccctgcctgagcgatggaaacagagcagaactgtccatgcttctggggactctctggggcacgtgtctggcccagggagacctgagctcctcccgctgaggaccgtctccgagtccgtgcagaggaataagcgggactgtctcgtgcgacgatgtagccagccggtctttgaggctgaccaattccaatatgccaaagaatcgtatatttaggagggaggccatacgccatgccatagcttgtgctatctgtaaatatgagacttgtaaagaactgcctgtagattgtttttaaaaggtcttgaataagctccttgagaaagttgtggaaagccctcctcagtgaggatagctacaccatggccatggcgcatcagatagtctctgtgtacctggatttgtgcaatatgtaaaaatgtatcaaatgtattatagataaggtgttaggtgcaaaggatgtctaataatccctgcacacgttttgaacttgcagtgaagtacactgctgttccttgcttcctggggcacttttctcttggttagtgtttaaaaattatcttcgcttttttaatgtggcctcaaatgtcatgccaattttcacatcttccacaaactccatttagggagaaatgtttaaatctctggtataagtttactccataccagagtaaactatatattactctatataagcagtcttgcaataactaatcaccaccatagaagaaagaaacagactgcaaggaacagagttgagtgtctggagtcatcaaaggcattaaaaactccagtaaaagctggggccgtagcaaaaatcatgaaaaacacttcaacgtgtcctttcaatcatccaattaaatgtgggtagattaatgaaaatgtattacatcaatattaactcat

SEQ ID NO: 2 - DNA, 3051 bp, Homo sapiens
gacatagctgccctaaaaggaatgaggaagcgagagctctccagtgtctggctggctccgtccgtgtgacagcccatgatgttctttccggtctctgtaatattctgaatttccacctgcccgccccttcgcttataatgcagagcatgtgaagggagaccggctcggtctctctctctcccagtggactagaaggagcagagagttatgctgtttctcccattctttacagctcaccggatgtaaaagaactctggctagagaccctccaaggacagaggcacagccacacgggagtgaaatccacccctggacagtcagccgcaatactgatgaagctgagaagcagccacaatgcttcaaaaacactaaacgccaataatatggagacactaatcgaatgtcaatcagagggtgatatcaaggaacatcccctgttggcatcatgtgagagtgaagacagtatttgccagctcattggacattctcactattctatgccttaaaggcccttcaacggaagggatattcaggagagcagccaacgagaaagcccgtaaggagctgaaggaggagctcaactctggggatgcggtggatctggagaggctccccgtgcacctcctcgctgtggtctttaaggacttcctcagaagtatcccccggaagctactttcaagcgacctctttgaggagtggatgggtgctctggagatgcaggacgaggaggacagaatcgaggccctgaaacaggttgcagataagctcccccggcccaacctcctgctactcaagcacttggtctatgtgctgcacctcatcagcaagaactctgaggtgaacaggatggactccagcaatctggccatctgcattggacccaacatgctcaccctggagaatgaccagagcctgtcatttgaagcccagaaggacctgaacaacaaggtgaagacactggtggaattcctcattgataactgctttgaaatatttggggagaacattccagtgcattccagtatcacttctgatgactccctggagcacactgacagttcagatgtgtcgaccctgcagaatgactcagcctacgacagcaacgaccctgatgtggaatccaacagcagcagtggcatcagctctcccagcaggcagccccaggtgcccatggccacagctgctggcttggatagcgcgggcccacaggatgcccgagaggtcagcccagagcccattgtgagcaccgtggccaggctgaaaagctccctcgcacagcccgataggagatactcagagcccagcatgccatcctcccaggagtgcctcgagagccgggtgacaaaccaaacactaacaaagagtgaaggggacttccccgtgccccgggtaggctctcgtttggaaagtgaggaggctgaagacccatttccagaggaggtcttccctgcagtgcaaggcaaaaccaagaggccggtggacctgaagatcaagaacttggccccgggttcggtgctcccgcgggcactggttctcaaagccttctccagcagctcgctggacgcgtcctctgacagctcgcccgtggcttctccttccagtcccaaaagaaatttcttcagcagacatcagtctttcaccacaaagacagagaaaggcaagcccagccgagaaattaaaaagcactccatgtctttcacctttgcccctcacaaaaaagtgctgaccaaaaacctcagcgcgggctctgggaaatcgcaagactttaccagggaccacgtcccgaggggtgtcagaaaggaaagccagcttgccggccgaatcgtgcaggaaaatgggtgtgaaacccacaaccaaacagcccgcggcttctgcctgagaccccacgccctctcggtggatgatgtgttccagggagctgactgggagaggcctggaagcccaccctcttatgaagaggccatgcagggcccggcagccagactagtggcctccgagagccagaccgtggggagcatgacggtggggagcatgagggcgaggatgctggaggcgcactgcctcctaccccctcttccacctgctcaccacgtagaggactcaagacacaggggcagcaaagagccactccctggccacggactctctcccctgcctgagcgatggaaacagagcagaactgtccatgcttctggggactctctggggcacgtgtctggcccagggagacctgagctcctcccgctgaggaccgtctccgagtccgtgcagaggaataagcgggactgtctcgtgcgacgatgtagccagccggtctttgaggctgaccaattccaatatgccaaagaatcgtatatttaggagggaggccatacgccatgccatagcttgtgctatctgtaaatatgagacttgtaaagaactgcctgtagattgtttttaaaaggtcttgaataagctccttgagaaagttgtggaaagccctcctcagtgaggatagctacaccatggccatggcgcatcagatagtctctgtgtacctggatttgtgcaatatgtaaaaatgtatcaaatgtattatagataaggtgttaggtgcaaaggatgtctaataatccctgcacacgttttgaacttgcagtgaagtacactgctgttccttgcttcctggggcacttttctcttggttagtgtttaaaaattatcttcgcttttttaatgtggcctcaaatgtcatgccaattttcacatcttccacaaactccatttagggagaaatgtttaaatctctggtataagtttactccataccagagtaaactatatattactctatataagcagtcttgcaataactaatcaccaccatagaagaaagaaacagactgcaaggaacagagttgagtgtctggagtcatcaaaggcattaaaaactccagtaaaagctggggccgtagcaaaaatcatgaaaaacacttcaacgtgtcctttcaatcatccaattaaatgtgggtagattaatgaaaatgtattacatcaatattaactcat

SEQ ID NO: 3 - protein, 731 aa, Homo sapiens
MKLRSSHNASKTLNANNMETLIECQSEGDIKEHPLLASCESEDSICQLIEVKKRKKVLSWPFLMRRLSPASDFSGALETDLKASLFDQPLSIICGDSDTLPRPIQDILTILCLKGPSTEGIFRRAANEKARKELKEELNSGDAVDLERLPVHLLAVVFKDFLRSIPRKLLSSDLFEEWMGALEMQDEEDRIEALKQVADKLPRPNLLLLKHLVYVLHLISKNSEVNRMDSSNLAICIGPNMLTLENDQSLSFEAQKDLNNKVKTLVEFLIDNCFEIFGENIPVHSSITSDDSLEHTDSSDVSTLQNDSAYDSNDPDVESNSSSGISSPSRQPQVPMATAAGLDSAGPQDAREVSPEPIVSTVARLKSSLAQPDRRYSEPSMPSSQECLESRVTNQTLTKSEGDFPVPRVGSRLESEEAEDPFPEEVFPAVQGKTKRPVDLKIKNLAPGSVLPRALVLKAFSSSSLDASSDSSPVASPSSPKRNFFSRHQSFTTKTEKGKPSREIKKHSMSFTFAPHKKVLTKNLSAGSGKSQDFTRDHVPRGVRKESQLAGRIVQENGCETHNQTARGFCLRPHALSVDDVFQGADWERPGSPPSYEEAMQGPAARLVASESQTVGSMTVGSMRARMLEAHCLLPPLPPAHHVEDSRHRGSKEPLPGHGLSPLPERWKQSRTVHASGDSLGHVSGPGRPELLPLRTVSESVQRNKRDCLVRRCSQPVFEADQFQYAKESYI

SEQ ID NO: 4 - protein, 553 aa, Homo sapiens
MGALEMQDEEDRIEALKQVADKLPRPNLLLLKHLVYVLHLISKNSEVNRMDSSNLAICIGPNMLTLENDQSLSFEAQKDLNNKVKTLVEFLIDNCFEIFGENIPVHSSITSDDSLEHTDSSDVSTLQNDSAYDSNDPDVESNSSSGISSPSRQPQVPMATAAGLDSAGPQDAREVSPEPIVSTVARLKSSLAQPDRRYSEPSMPSSQECLESRVTNQTLTKSEGDFPVPRVGSRLESEEAEDPFPEEVFPAVQGKTKRPVDLKIKNLAPGSVLPRALVLKAFSSSSLDASSDSSPVASPSSPKRNFFSRHQSFTTKTEKGKPSREIKKHSMSFTFAPHKKVLTKNLSAGSGKSQDFTRDHVPRGVRKESQLAGRIVQENGCETHNQTARGFCLRPHALSVDDVFQGADWERPGSPPSYEEAMQGPAARLVASESQTVGSMTVGSMRARMLEAHCLLPPLPPAHHVEDSRHRGSKEPLPGHGLSPLPERWKQSRTVHASGDSLGHVSGPGRPELLPLRTVSESVQRNKRDCLVRRCSQPVFEADQFQYAKESYI

SEQ ID NO: 5 - DNA, 3612 bp, Homo sapiens
agaggcaggcggcttgtgagacgggctccagagaaaggacctccctgggtctctcatttcctggctgaagtttctcttctcgctgctgtggcagcatccaacccacacacacaggacccgcatcctgggtgatgaagtcagacacgcagcagctgggtgagtgctaacgctcagataagcatctgtgccattgtggggactccctgggctgctctgcacccggacacttgctctgtccccgccatgtacaacgggtcgtgctgccgcatcgagggggacaccatctcccaggtgatgccgccgctgctcattgtggcctttgtgctgggcgcactaggcaatggggtcgccctgtgtggtttctgcttccacatgaagacctggaagcccagcactgtttaccttttcaatttggccgtggctgatttcctccttatgatctgcctgccttttcggacagactattacctcagacgtagacactgggcttttggggacattccctgccgagtggggctcttcacgttggccatgaacagggccgggagcatcgtgttccttacggtggtggctgcggacaggtatttcaaagtggtccacccccaccacgcggtgaacactatctccacccgggtggcggctggcatcgtctgcaccctgtgggccctggtcatcctgggaacagtgtatcttttgctggagaaccatctctgcgtgcaagagacggccgtctcctgtgagagcttcatcatggagtcggccaatggctggcatgacatcatgttccagctggagttctttatgcccctcggcatcatcttattttgctccttcaagattgtttggagcctgaggcggaggcagcagctggccagacaggctcggatgaagaaggcgacccggttcatcatggtggtggcaattgtgttcatcacatgctacctgcccagcgtgtctgctagactctatttcctctggacggtgccctcgagtgcctgcgatccctctgtccatggggccctgcacataaccctcagcttcacctacatgaacagcatgctggatcccctggtgtattatttttcaagcccctcctttcccaaattctacaacaagctcaaaatctgcagtctgaaacccaagcagccaggacactcaaaaacacaaaggccggaagagatgccaatttcgaacctcggtcgcaggagttgcatcagtgtggcaaatagtttccaaagccagtctgatgggcaatgggatccccacattgttgagtggcactgaacaagcagaccaacaacactgaggaagatagagtggtgacttagaattaactcgtgctaaggggtcgggggctttgaaaatgccacccccctttcttattgcaagacggcttctcgcacatgaactgcatccttctcattctgtcggaaatgaaattcacacaactataccttttggggaggttccagttgattgaagtgagttggctgcattttcttatctgatcacaatggcaggggacagaatgtgcatggagtggagcatgtgtgtgttgggaggggggctaggaactgcacagcccttgtgtaattttcgttgtttgtttttgttttgagacagagtctcactctgtgtcccaggctggagtgcagtggcacagtctcggctcactgcaacctctgcctcccgggttcaagcaattctcctgcctcagcctcccgagtagctgggattagaggcgccagccaacacacccggctaatttttgtatttttagtagagacagggttttgccatgttggccaggctggtctcgagctcctgacctcaggtgatccgcctgccttggcctcccaaagtggtgggatcacaggcgtgagccaccgtgcccggcctcccctgtgtcattttaaatggctaagtaaatgggtatatgtgtttgaatggggcatgttcactctcttaggggctatggggcagttagcagcatttcctatcctctgaccttaaatcattccttatctcagaaaacagaaaccgggctcagtcaatcaatgctttatttcaggccgaatgaggctctttagattgggatctattgatctatcaattttcatctttacatttctttgtacatctgtacattttgtccaaatgtacatctgtacgtctgtcatcattgtgacttcctggtagcccaagaagaacaacaacaaaacaatctgctctgaccttcttcaaatctttgtatttcaaagaaggtgctgagggatctgtttccttgccctggcttctccagtgggatgtgctgagtccaatacaattgcttttataattgcttttgacaacttgtcatgtgactgtgaattgaaattattcacttattttccaagtatttactgaattcgtatttggtggcaggcagtatactgtgtaatttttagtggagggtcattagtcaactcttatgtgacagtaaagttttttgggggggtggggacagagaagttaagagctttcatcctttcacggaatacagtttctagaccgattctgtgtgaacatcagttttgtcctcttattgcaagactccctcatacacatgagtttcccaaatgtgtacctggacccctcgaaacagaggactctacgaaatgacaggctgcccctgccctgaattagggggaaacattccaggccaactctagctcctttctcaagctacaaagtggtgaacatggttctcaactccttaatttatactctctcaaatgcccaggatactctacccacttaagaaccttgccaacttctgggggttgggcatggtggctcgcgcttgtgatcccagcactttgggagactgaggcggatcacctgaggtcaggagttctagaccagcctgaccgacatggagaaacctcgtctctactgaaaattcaaaattagcctggtgtggtggcgcatgcctatagtctcagcctccagagtagctgggactgcgggcgccccaccaccacgcccggctaattttttgtatttttagtacagacggggtttcattgtgttagccgggatggtcttgatctcctgacttgtgatccgcctgcctcggcctcccaaagtgcttggattacaggtgtaagccaccgcaccccgcccagcctggcagattttatttaatcatttgtagcttcattttcctcgtctgtcaaacagggatactgtaatacaacctcagtgtgtcattgggcagtttaaatgaatgtacattcctgaggcatcagaactttgttcactgttatatacccaatgcctagaagaggacctgcacatagcaggtgctcagtaaatgtttgttgaatgaatgattaagtgcatgtaaagcattaagcatagcgcctggcagtaagtgctcaatattatgacttcttatattaacacgttttacatataaagaaatggaggcaagaaagcatttcctttggggtttagagcgcttaagttgttcctctgttatcatgcctgaattcccccgcccctcagttacctggggaagagtaaaggcaagaattcttaccagcattagtcatacatcctcctgatagg

SEQ ID NO: 6 - DNA, 2345 bp, Homo sapiens
agaggcaggcggcttgtgagacgggctccagagaaaggacctccctgggtctctcatttcctggctgaagtttctcttctcgctgctgtggcagcatccaacccacacacacaggacccgcatcctgggtgatgaagtcagacacgcagcagctgggtgagtgctaacgctcagataagcatctgtgccattgtggggactccctgggctgctctgcacccggacacttgctctgtccccgccatgtacaacgggtcgtgctgccgcatcgagggggacaccatctcccaggtgatgccgccgctgctcattgtggcctttgtgctgggcgcactaggcaatggggtcgccctgtgtggtttctgcttccacatgaagacctggaagcccagcactgtttaccttttcaatttggccgtggctgatttcctccttatgatctgcctgccttttcggacagactattacctcagacgtagacactgggcttttggggacattccctgccgagtggggctcttcacgttggccatgaacagggccgggagcatcgtgttccttacggtggtggctgcggacaggtatttcaaagtggtccacccccaccacgcggtgaacactatctccacccgggtggcggctggcatcgtctgcaccctgtgggccctggtcatcctgggaacagtgtatcttttgctggagaaccatctctgcgtgcaagagacggccgtctcctgtgagagcttcatcatggagtcggccaatggctggcatgacatcatgttccagctggagttctttatgcccctcggcatcatcttattttgctccttcaagattgtttggagcctgaggcggaggcagcagctggccagacaggctcggatgaagaaggcgacccggttcatcatggtggtggcaattgtgttcatcacatgctacctgcccagcgtgtctgctagactctatttcctctggacggtgccctcgagtgcctgcgatccctctgtccatggggccctgcacataaccctcagcttcacctacatgaacagcatgctggatcccctggtgtattatttttcaagcccctcctttcccaaattctacaacaagctcaaaatctgcagtctgaaacccaagcagccaggacactcaaaaacacaaaggccggaagagatgccaatttcgaacctcggtcgcaggagttgcatcagtgtggcaaatagtttccaaagccagtctgatgggcaatgggatccccacattgttgagtggcactgaacaagcagaccaacaacactgaggaagatagagtggtgacttagaattaactcgtgctaaggggtcgggggctttgaaaatgccacccccctttcttattgcaagacggcttctcgcacatgaactgcatccttctcattctgtcggaaatgaaattcacacaactataccttttggggaggttccagttgattgaagtgagttggctgcattttcttatctgatcacaatggcaggggacagaatgtgcatggagtggagcatgtgtgtgttgggaggggggctaggaactgcacagcccttgtgtaattttcgttgtttgtttttgttttgagacagagtctcactctgtgtcccaggctggagtgcagtggcacagtctcggctcactgcaacctctgcctcccgggttcaagcaattctcctgtctcagcctccagagtagctgggactacgggcgccccaccaccacgcccggctaattttttgtatttttagtacagacggggtttcattgtgttagccgggatggtcttgatctcctgacttgtgatccgcctgcctcggcctcccaaagtgcttggattacaggtgtaagccaccgcaccccgcccagcctggcagattttatttaatcatttgtagcttcattttcctcgtctgtcaaacagggatactgtaatacaacctcagtgtgtcattgggcagtttaaatgaatgtacattcctgaggcatcagaactttgttcactgttatatacccaatgcctagaagaggacctgcacatagcaggtgctcagtaaatgtttgttgaatgaatgattaagtgcatgtaaagcattaagcatagcgcctggcagtaagtgctcaatattatgacttcttatattaacacgttttacatataaagaaatggaggcaagaaagcatttcctttggggtttagagcgcttaagttgttcctctgttatcatgcctgaattcccccgcccctcagttacctggggaagagtaaaggcaagaattcttaccagcattagtcatacatcctcctgatagg

SEQ ID NO: 7 - protein, 346 aa, Homo sapiens
MYNGSCCRIEGDTISQVMPPLLIVAFVLGALGNGVALCGFCFHMKTWKPSTVYLFNLAVADFLLMICLPFRTDYYLRRRHWAFGDIPCRVGLFTLAMNRAGSIVFLTVVAADRYFKVVHPHHAVNTISTRVAAGIVCTLWALVILGTVYLLLENHLCVQETAVSCESFIMESANGWHDIMFQLEFFMPLGIILFCSFKIVWSLRRRQQLARQARMKKATRFIMVVAIVFITCYLPSVSARLYFLWTVPSSACDPSVHGALHITLSFTYMNSMLDPLVYYFSSPSFPKFYNKLKICSLKPKQPGHSKTQRPEEMPISNLGRRSCISVANSFQSQSDGQWDPHIVEWH

SEQ ID NO: 8 - DNA, 3748 bp, Homo sapiens
ggctgaggcggaggagccgccgcttctgacctcgctctggctccggtgcgcgcggctgagcgcgtgcgaggccccgcgcgtcgggcaggggcggcggcggccactgcgcgccgccctgaggagcgccccagcggcggcgcgactgcggctgaggagagagccggctccgggcctccgcgtcctcctgctcccccggccccccgcctcctcgggggggcggcggcggcgatgttctcggtcctctcgtacgggcggctggtggcccgcgccgtgctcggcggcctctcgcagaccgaccccagggccggcggcggcggcggcggcgactacggactggtgacggccggctgcggcttcgggaaggacttccgtaagggcctcctcaagaagggcgcgtgctacggggacgacgcgtgcttcgtggcccggcaccgttccgcggacgtgctcggggttgcagatggtgtaggaggctggagagactatggagttgatccatctcaattctcagggactttaatgcggacgtgtgaacgtttagtaaaagaaggacggttcgtacctagtaatcccattggaattctcaccacaagctactgtgagttgctgcaaaataaagtccctttgctcggtagcagcaccgcctgcattgtggtgctggacagaaccagccaccgcttacacacagcaaacctgggcgattcaggcttcctggttgtcaggggtggtgaagtcgtgcaccgatcagatgagcagcagcattacttcaacactccattccagctctcaatcgctccccctgaagccgagggagtcgtcttgagcgacagtccggatgctgctgatagcacgtctttcgatgtccagctaggagacattatcctgacggcaacagatggactctttgacaacatgcctgattatatgattcttcaggagctaaaaaagttaaagaattcaaattatgagagtatacaacagactgccagaagcattgctgagcaagctcatgagctggcctatgacccaaattatatgtcaccttttgcacagtttgcatgtgacaatggattgaatgtgagaggtggaaagccagatgacatcaccgtccttctttcaatagtggctgagtatacagactagctgaggtgtcaagtcctgcctttcctttcatcatcccaaatttcccctgccatgtgtgctgatcctgctggcaggaccacatttctttgccactgatctcaatggccagtgatgtaagtcttttgcctgtcttcttgagactcgttgagatctttgttgagaaccactactatcattcactagctcatatctgccggcagcaattgaagagatccaatatttgaagattggccttcatttctcgatgttctttccatgatggggatggaggtgttcagtgccaccgtggctgttacttttcaaagtagttgaagtattgaaaatgagtaatgttggtaaagtgaattcaaaatcctagtatgctaaagggatggtacaagtctaacacaaattgtacgtaatgatacatctactagaaacatacattattcatcaaaagaaatgttacatgtgtactccacaggcatagtctttgttatgatgattggtgtggctttatgtctttgttataaactcctatttttcaggggcttatgattctgctctaaaacattgctctgggttatacagttttgatcccaaaagcttttttgttacaaatcgggagaaaaatccattttagttctatggatggaaatatttcatgcttttaaaaagatgtttgtgttcctgtggttaaagttttggcagtttattgattagtccaaatcacaggctaaggcctgatctccaggaggggtaggggagacactttaccagtatttttttatggaaataatactcaaggttgtaaaacccctcaaagcctagaaatttaattgttatggctgaaattcctcctagttgtctgatagaatgcccctgaatgggaactctaggtcccaaggcctgaagggttgagaacagacagctgtaactttgaattttgttggctttcagtggtcatgctacctacccatactcgtactctcagaccttttattagtagccttgctttctatagagcatgcaccaaatccagtgagtccatgtggagagagcactgtgtgcgcagcggcagcagcacagacgtccatgaggaaaactcccagtgatgatctgacatttacaattaccccacatggaaatttaggggtttctgaatcaagcttaatgtttacagtttccaaatagccattttgcagtgtatagtttccttacaaaactaccccgcattcagttttcacattatctgcaagctgaaacttatttttaagttttgtgtacaagttgactgctgtaaagatatatatttttgggtcagtttttttccttcattaacttggtggtagaaaaaaatatatacttagaaatccttaaattaaagccatgttttatatataagtcaggtaacattggtgtatagatgagaatgcaattaaacctgatgagaatctacttgagaatatagaaagtctttctctaaaggagatactgactccctggtttattgcattaaaatttatgtttgaggttacctcaacttgttttaaaagattttgttttgtgaatttgtactgtatatttgagtaactgtcaggcttttatttaaaattgtttaacatgtaccatgtacatgtcattactatatttcaatgcatcatgcttgtaacaggcatttcatttataataagaatgagttattcatttgtaagccgttcagtaatttatctactattcctaaattggcataatgttagataatctattttgaatcacctttaattacatgtcagaatgccttaactaccctaacttgacaaaacagaattctttggtagacgcggtgggggcggggtggggggtctggacggagtctctatttaaggagaaatcatcatgctatgataaaacacagaagcatgagtggcaagtggcggggtatttattttgcacaaactatttgcagtctctgtgtatttaaaaagtaaagaaagttgcatccagaagggttttgttagaatgaatacatttatattaggactgacaacttcagctcttttgtttaggttttcaattatttttggtaagagtatgtagccttatgatctggatatattttgcattcattttccaacgcctacatttaattcctggtaagagcagtgctcgtcaagtttctggtttttctctgctctcatttaacccgtcaaacacaatctttgtaaagctagattggtggtgttttatacaacttatttactcagcttacctttttgagaaacgattgttagaaattgacgatgtgtttgttccagtgatactgaaagtagtgggggcaagaattgagtttcacagtggaattggctttggatctggcctatagattagtgacataaaatattttctctattttcccctgttctttttgtgttatgcacttaattttatgactgccgggggggtcagctggagtgctgcttaacaagtatctctcctactctcagtggtcagaggctgtgttggacccatagtagaattttccaggtcacagacccaagcttccatgggttgttactgtgctgtaccacttggtgggtctgattctgaacctgatgtgtgtgtt

SEQ ID NO: 9 - protein, 304 aa, Homo sapiens
MFSVLSYGRLVARAVLGGLSQTDPRAGGGGGGDYGLVTAGCGFGKDFRKGLLKKGACYGDDACFVARHRSADVLGVADGVGGWRDYGVDPSQFSGTLMRTCERLVKEGRFVPSNPIGILTTSYCELLQNKVPLLGSSTACIVVLDRTSHRLHTANLGDSGFLVVRGGEVVHRSDEQQHYFNTPFQLSIAPPEAEGVVLSDSPDAADSTSFDVQLGDIILTATDGLFDNMPDYMILQELKKLKNSNYESIQQTARSIAEQAHELAYDPNYMSPFAQFACDNGLNVRGGKPDDITVLLSIVAEYTD

SEQ ID NO: 10 - DNA, 1736 bp, Homo sapiens
aaaatttgctgattaaatgaatgtgggtgtgtttgagagggatcctagacagccaagccttctggcatgaaacgctgagaagatgggagtgtctgctggcagagatgaaagtgagcaggggtgagcgcagccactgcccaacgcaaaccgtgaagaagcttctggaagagcagaggcgccgccagcagcagcagcccgacgctggcggggtgcagggacaatttctccctcccccagagcagcccctgaccccatctgtgaatgaggctgtgactggccaccctcccttcccagcacactcggagactgtgggttctggacctagcagcctgggctttccagactgggaccccaacacgcatgctgcctacactgacagcccctactcttgccctgcttctgctgccgaaaatttcctgcctcctgacttctacccaccctcggacccagggcagccgtgcccatttccccagggcatggaggctggaccctggagagtttctgcacccccttcaggacccccacagttccccgctgtggtccctggaccatcgctggaggtggcccgagctcacatgctggctttggggccacagcagctgctggcccaggatgaggagggggacacgctccttcacctgtttgcggctcgggggctgcgctgggcggcatatgctgcggctgaggtgctccaggtgtaccggcgtcttgacattcgtgagcataagggcaagacccctctcctggtggcggctgctgccaaccagcccctgattgtggaggatctgttgaacctgggagcagagcccaatgccgctgaccatcagggacgttcggtcttgcacgtggccgctacctacgggctcccaggagttctcttggctgtgcttaactctggggtccaggttgacctggaagccagagacttcgagggcctcaccccgctccacacggccatcctggcccttaacgttgctatgcgcccttccgacctctgtccccgggtgctgagcacacaggcccgagacaggctggattgtgtccacatgttgctgcaaatgggtgctaatcacaccagccaggagatcaagagcaacaagacagttctgcacttggccgtgcaggctgccaaccccactctggttcagctgctgctggagctgccccggggagacctgcggacctttgtcaacatgaaggcccacgggaacacagccctccacatggcggctgccctgccccctgggccggcccaggaggccatcgtgcggcacctgttggcagctggggcggaccccacactgcgcaacctggagaatgagcagcccgttcacctgctgcggcccgggccgggccctgaggggctccggcagctgttgaagaggagccgtgtggcgccgccaggcctgtcctcttaggactcaaacccagaccctggactgattttccagtccccaccgtcctgcgggacagccagcgtatgctaatgttgcaaacccatgataatgtatgtggaatatcctgccattggggttttacattaaaaccccagaatggctgcagaggggtgaacaggccccaatatttggggtgctgtgatacccctcttctacccacaaggagccctcttgatgatttctgtgaaatcgaggccccttgattgtttctgtgaaacaccctgcacccctagtcctttccccactgagatctttcgggttctctcccctaactcagct

SEQ ID NO: 11 - protein, 465 aa, Homo sapiens
MWVCLRGILDSQAFWHETLRRWECLLAEMKVSRGERSHCPTQTVKKLLEEQRRRQQQQPDAGGVQGQFLPPPEQPLTPSVNEAVTGHPPFPAHSETVGSGPSSLGFPDWDPNTHAAYTDSPYSCPASAAENFLPPDFYPPSDPGQPCPFPQGMEAGPWRVSAPPSGPPQFPAVVPGPSLEVARAHMLALGPQQLLAQDEEGDTLLHLFAARGLRWAAYAAAEVLQVYRRLDIREHKGKTPLLVAAAANQPLIVEDLLNLGAEPNAADHQGRSVLHVAATYGLPGVLLAVLNSGVQVDLEARDFEGLTPLHTAILALNVAMRPSDLCPRVLSTQARDRLDCVHMLLQMGANHTSQEIKSNKTVLHLAVQAANPTLVQLLLELPRGDLRTFVNMKAHGNTALHMAAALPPGPAQEAIVRHLLAAGADPTLRNLENEQPVHLLRPGPGPEGLRQLLKRSRVAPPGLSS

SEQ ID NO: 12 - DNA, 1834 bp, Homo sapiens
ttcgccggagcgcgacccggggactcccaggcctgtgggcgggccctgcccaggactgggcggtgccataacccctagtttaaaaactcgcgggtaccggacccaagatcggggacccggcggcggctccgcgggggaaacagcgaggctggcgcagcgccgaggccgcggccctgggggcccgcaatccacgccacggaatccccgagtgagcaggggtgagcgcagccactgcccaacgcaaaccgtgaagaagcttctggaagagcagaggcgccgccagcagcagcagcccgacgctggcggggtgcagggacaatttctccctcccccagagcagcccctgaccccatctgtgaatgaggctgtgactggccaccctcccttcccagcacactcggagactgtgggttctggacctagcagcctgggctttccagactgggaccccaacacgcatgctgcctacactgacagcccctactcttgccctgcttctgctgccgaaaatttcctgcctcctgacttctacccaccctcggacccagggcagccgtgcccatttccccagggcatggaggctggaccctggagagtttctgcacccccttcaggacccccacagttccccgctgtggtccctggaccatcgctggaggtggcccgagctcacatgctggctttggggccacagcagctgctggcccaggatgaggagggggacacgctccttcacctgtttgcggctcgggggctgcgctgggcggcatatgctgcggctgaggtgctccaggtgtaccggcgtcttgacattcgtgagcataagggcaagacccctctcctggtggcggctgctgccaaccagcccctgattgtggaggatctgttgaacctgggagcagagcccaatgccgctgaccatcagggacgttcggtcttgcacgtggccgctacctacgggctcccaggagttctcttggctgtgcttaactctggggtccaggttgacctggaagccagagacttcgagggcctcaccccgctccacacggccatcctggcccttaacgttgctatgcgcccttccgacctctgtccccgggtgctgagcacacaggcccgagacaggctggattgtgtccacatgttgctgcaaatgggtgctaatcacaccagccaggagatcaagagcaacaagacagttctgcacttggccgtgcaggctgccaaccccactctggttcagctgctgctggagctgccccggggagacctgcggacctttgtcaacatgaaggcccacgggaacacagccctccacatggcggctgccctgccccctgggccggcccaggaggccatcgtgcggcacctgttggcagctggggcggaccccacactgcgcaacctggagaatgagcagcccgttcacctgctgcggcccgggccgggccctgaggggctccggcagctgttgaagaggagccgtgtggcgccgccaggcctgtcctcttaggactcaaacccagaccctggactgattttccagtccccaccgtcctgcgggacagccagcgtatgctaatgttgcaaacccatgataatgtatgtggaatatcctgccattggggttttacattaaaaccccagaatggctgcagaggggtgaacaggccccaatatttggggtgctgtgatacccctcttctacccacaaggagccctcttgatgatttctgtgaaatcgaggccccttgattgtttctgtgaaacaccctgcacccctagtcctttccccactgagatctttcgggttctctcccctaactcagct

SEQ ID NO: 13 - protein, 313 aa, Homo sapiens
MEAGPWRVSAPPSGPPQFPAVVPGPSLEVARAHMLALGPQQLLAQDEEGDTLLHLFAARGLRWAAYAAAEVLQVYRRLDIREHKGKTPLLVAAAANQPLIVEDLLNLGAEPNAADHQGRSVLHVAATYGLPGVLLAVLNSGVQVDLEARDFEGLTPLHTAILALNVAMRPSDLCPRVLSTQARDRLDCVHMLLQMGANHTSQEIKSNKTVLHLAVQAANPTLVQLLLELPRGDLRTFVNMKAHGNTALHMAAALPPGPAQEAIVRHLLAAGADPTLRNLENEQPVHLLRPGPGPEGLRQLLKRSRVAPPGLSS

SEQ ID NO: 14 - DNA, 3049 bp, Homo sapiens
taacgagcttccttccaccgcaaaagagctggagaacaatgctaggcaacgtgctggagaccttggccctcggaacccaagtcacgcctcccatgtgagctctggagggagaactttatgtgttgcactgagggcagtctccggaaacgcgattcgcagcgggcgccggaagcggtgttgtgtctgcagctctggcagaggactgttccactagacacgctgaagggactgggtacgtgttttccttcaggaccagagctgagaggagctgggatcgcggcggcaatggaacgggcctcagaaaggcgcacggccagcgcgctttttgcggggttccgggccttgggacttttcagcaacgacattccacacgtggtgcggttcagcgcgctcaagcgccggttctatgtaacaacctgcgtgggcaagagtttccacacctatgacgttcagaaacttagtctggttgcagtaagtaattctgttccacaggatatctgctgtatggcagctgatggcagattagtctttgctgcttatggaaatgttttctctgcatttgcccgtaataaagagatagtacatacctttaagggtcataaggcagaaatccatttcttgcaaccctttggagaccacattatctctgttgatactgatggcattcttattatttggcacatatattcagaagaagaatacctgcagttgacttttgataaatcagtatttaaaatttctgcaattttgcatccaagtacctacttgaataaaatacttctgggcagtgaacaaggaagcctgcagttgtggaatgtaaaatccaataaacttctatatacatttccaggatggaaagttggagtgacagctcttcagcaggcaccagccgtggatgttgttgctattggtcttatgtcaggtcaagttatcattcacaacattaaatttaatgaaacattaatgaagtttcgtcaagactggggacccattacttcaatttcatttcgcacagatggtcatccagtaatggcagctggaagcccatgtggccatattggactctgggatctagaagacaaaaaattaatcaaccaaatgagaaatgcacactctacagcaattgccggactgacatttctccatagagagccacttcttgtcacaaatggcgctgacaatgctcttaggatatggatatttgatggtcctacaggtgaaggccgacttttgagattcagaatgggtcatagtgctcctcttaccaatatcagatattatggacagaatggacagcagattctaagtgcaagtcaagatggaactcttcagtcattttccacggtacatgaaaaattcaataagagcttgggacatggattaataaataaaaagagagttaaacgtaaaggacttcagaataccatgtcagtgagacttccacccatcacaaagtttgcagcagaggaagctcgtgaaagtgactgggatggtatcattgcttgccatcaaggtaagctatcttgctcaacctggaattatcagaaatctacaataggcgcttactttctcaagccaaaagagttgaagaaagatgacataactgcaacagcagtggatataacttcttgtggaaactttgctgtaattggcctctcatcaggaactgtagatgtatataacatgcagtctggcatacatcgaggaagttttggcaaggatcaagctcacaagggatctgttagaggtgtcgcagtggatggattaaaccagttgacagttacaactggtagtgaaggattactcaaattctggaactttaaaaacaaaattttaatccattctgtgagcctcagttcatctccaaatatcatgttgctacatagggacagtggcattctgggactcgccttggatgacttctccattagtgttctggacatagaaactaggaagattgtcagagagttttctggacaccaaggccaaataaatgacatggcttttagtcctgatggtcgttggttaataagtgctgcgatggattgctctattaggacttgggaccttccttctgggtgccttatagactgctttttgttggactcggctcctctcaatgtttctatgtctcctactggagactttctggcaacttcccatgtggaccaccttggaatttatctatggtccaatatttccctgtattcagttgtttcattacggccacttcctgcagattatgtcccttcaatagtcatgcttcctggtacttgtcaaacccaagatgtagaagtatcagaagaaacagtagaaccaagtgatgaattgatagaatatgattcgccagaacagttgaatgagcaattggtgactctttcacttcttcctgaatcacgatggaaaaaccttcttaaccttgatgttattaagaaaaagaataaaccaaaggaaccacccaaagtacccaaatcagcaccatttttcattccaacaattcctggccttgtacccagatatgctgcacctgaacaaaataatgatccccagcagtctaaagtggtaaatcttggagttttggctcaaaaatcagatttctgcttgaaacttgaagaaggactggtaaataataagtatgacactgctctcaaccttctgaaagaatcaggcccatcaggaattgaaacagagctgcgaagcttgtctcctgattgtggtgggtccatagaagttatgcagagcttcttgaaaatgattgggatgatgctggacagaaagcgtgattttgagttagcccaggcataccttgcattgtttctaaagttacaccttaaaatgcttccttcagagccagtactcctagaagaaataacaaatttgtcatcccaggtggaagaaaactggacccatttgcaatcactcttcaatcaaagcatgtgtattttaaattatctcaaaagtgctttgttgtaaaaataaatttgtgactaaacaaagactttcatattaaatgggttcaattgaactcatttcttattttccaagtgtc

SEQ ID NO: 15 - protein, 951 aa, Homo sapiens
MCCTEGSLRKRDSQRAPEAVLCLQLWQRTVPLDTLKGLGTCFPSGPELRGAGIAAAMERASERRTASALFAGFRALGLFSNDIPHVVRFSALKRRFYVTTCVGKSFHTYDVQKLSLVAVSNSVPQDICCMAADGRLVFAAYGNVFSAFARNKEIVHTFKGHKAEIHFLQPFGDHIISVDTDGILIIWHIYSEEEYLQLTFDKSVFKISAILHPSTYLNKILLGSEQGSLQLWNVKSNKLLYTFPGWKVGVTALQQAPAVDVVAIGLMSGQVIIHNIKFNETLMKFRQDWGPITSISFRTDGHPVMAAGSPCGHIGLWDLEDKKLINQMRNAHSTAIAGLTFLHREPLLVTNGADNALRIWIFDGPTGEGRLLRFRMGHSAPLTNIRYYGQNGQQILSASQDGTLQSFSTVHEKFNKSLGHGLINKKRVKRKGLQNTMSVRLPPITKFAAEEARESDWDGIIACHQGKLSCSTWNYQKSTIGAYFLKPKELKKDDITATAVDITSCGNFAVIGLSSGTVDVYNMQSGIHRGSFGKDQAHKGSVRGVAVDGLNQLTVTTGSEGLLKFWNFKNKILIHSVSLSSSPNIMLLHRDSGILGLALDDFSISVLDIETRKIVREFSGHQGQINDMAFSPDGRWLISAAMDCSIRTWDLPSGCLIDCFLLDSAPLNVSMSPTGDFLATSHVDHLGIYLWSNISLYSVVSLRPLPADYVPSIVMLPGTCQTQDVEVSEETVEPSDELIEYDSPEQLNEQLVTLSLLPESRWKNLLNLDVIKKKNKPKEPPKVPKSAPFFIPTIPGLVPRYAAPEQNNDPQQSKVVNLGVLAQKSDFCLKLEEGLVNNKYDTALNLLKESGPSGIETELRSLSPDCGGSIEVMQSFLKMIGMMLDRKRDFELAQAYLALFLKLHLKMLPSEPVLLEEITNLSSQVEENWTHLQSLFNQSMCILNYLKSALL

SEQ ID NO: 16 - DNA, 4617 bp, Homo sapiens
agatttaagtaagtcttccccaacaccgaatgggattccatcttcagacccagccagcgatgccatggaccccttccatgcttgcagtattcttaagcaactcaaaacaatgtacgatgaaggacagttgacagacattgtagtggaagtggatcacgggaaaacattttcctgtcatagaaacgttcttgctgcaatcagcccttacttcagatccatgttcactagcggccttacagaaagtactcaaaaagaagttcgaatagttggtgttgaagctgaatcgatggatttagtgttgaactatgcctacacttccagagttattcttacagaggccaatgttcaagccttgttcactgcagctagcatcttccagattccttccatccaagaccaatgtgctaagtatatgatcagtcatttggacccacagaattctattggggtctttatctttgctgatcattatggtcatcaggaactcggagatcgatcaaaagaatacattcgtaaaaagtttctgtgtgtcaccaaagaacaagagtttctccagttgacaaaagaccaactgataagtatactagacagtgacgatttaaatgtagaccgagaagagcatgtttatgaaagcattataaggtggtttgagcatgaacagaatgaaagagaagtgcaccttccagaaatttttgctaaatgcatacgttttcctctgatggaagatacctttatagagaaaattccacctcagtttgcacaggctatagccaaaagctgtgtagaaaagggaccatccaacaccaatggctgtacacagaggcttggaatgactgcttctgaaatgatcatatgttttgatgctgcccacaaacactcaggaaagaagcaaacagtgccttgtctagatatagtcacaggaagggtgtttaaactatgcaaaccaccaaatgacctgagagaagttgggattcttgtatcaccagataatgacatttacattgcaggagggtacaggccaagcagcagtgaggtctccatcgaccataaggcagaaaatgatttctggatgtatgatcattccaccaatagatggctatccaaaccatccttgcttcgagccagaataggctgcaaacttgtctattgctgtggtaaaatgtatgcaatcggaggtcgtgtttatgaaggtgatgggagaaactcactaaaatctgttgagtgctacgacagtagagagaattgttggacgactgtttgcgcgatgccagttgcaatggaatttcataatgctgtggagtacaaagagaagatctatgttttacagggagaattttttctcttctatgagcctcaaaaagactactggggtttcttaacccccatgactgtgcctagaatccagggcttagcagctgtatacaaggactctatctactacatagctggaacctgtggaaatcatcaacgtatgtttactgtagaagcctatgatattgagctaaataaatggactcgtaagaaagactttccatgtgatcagtccataaatccataccttaaactggtacttttccagaacaaactccatttatttgttcgagctactcaagtgactgttgaagaacacgtcttcagaaccagcagaaaaaattccctttaccaatatgatgacattgctgaccagtggatgaaagtgtatgagaccccagatcggctctgggaccttggccggcattttgaatgtgctgttgctaaactgtatcctcagtgtcttcagaaagtactctaaatgagtagcaggccttagtgcatcactggcatctcattcttaggaaacttgtctttgatacaaaagagtgctgacagtatttcagaaagctgagagagttttatacatggaaaatgggtatgcttaaagattgcagggtagggagggattttccttcatccttgtgacatttcatttcagtaaggaaaagataacaaagtgcaattatcagcatttttttttcctggcataaaattaatcatttcattttataattttgtgataaatagtaactgaggtaccagatgaatcaggacaactatgcactcttataagagcatttagggtattattgggtaaagacgtctaaacttgtttgatgtgacttttaattttaaatacgggtaacaatctgaggcaatatcactaggactttagctgtgacctctctaacacagagaagcactaacttagatcctcattcttaatatttatatgtatctatttttgtgtactgttttcaagtgtactgagatttaaatgtgttctattattagagtagatcgaagaaaaaattagtctcagaaagagcttttagtctgattgtttccatttcccatgtaattttaagttaagctaaagttttaaagtggcagttttctgtcgatgactttttcaagtgctaacactgtctcttttgtgaaaatctggaaaagtgctcatattcacaggtggctggtgctagtctaacttaattcatgtgtataactagatggatttaaatggtctgagcctatgcctatctttcaaattggtgtggatttcatggccatagtactttacctgttgaactcttgtgatttcacaagattctctacttatgtgataggagggtatggccagttattcatctaactggactcaatcttagaatagtaggaacattatacccagtttgcactaacatgggccatttgtagcccaaccttctcttccatctacctgtccattcattattggtacaaggaaaggtaacttatttctcttctgcacagagcataatgtgaagttttatacctacttttaaaattctgctttccagaaacaaaattcctgcagtggtctaatttaatgtctttaagtttcatattacaattaaaacctcattttttttttccatttttgcacttaacagtgatgaatacttttacgttggaatcctccttctagctgaaggtgattgaaaaggaaaagagtgagtgaacagaaccatagctttctaggtactaaagcattttttgcatttaactgatgaaatttctaacaatcatcagttaggaatattaacatgaaggataaaccaacttatttgtatacctaaggcaggcatttggatcagtaacatgttttactaagcctagagtaattcgtaaagggtataagcataggacagattttgccctcaatcacaatatttgtattcacttgaaagcaaactggcatggttcgtattttaaaaatcttgcacaaattgtaatgtgatactgtgaaacaaattgaaaacattgcctctttgcatcacatacctcgtttttcagaaactttccaaactgctttacatagacctctacaagtagggaatgttttctgaagcagaagttaaaatggacagcatttctagaattaacattttaaaatctagtcttagctagatatgtggtttcttcttattggtgttgatagtatgtctgtaatctctgtataaactttgtcaacatttttacctccccagttttatcttctgttttgtttttgtttttatcatcatgatgttttggagttattactgtgtattttagaaatcattctttacagttttgcattgctgaggagagagaaaaaacaatttttttgcaagagatgttcatgtaatttatttttgaaagctttgttgaataagatttcctgccgctttttgacaatcttgtgtatttagaaaaatgtattacttgaaaacatgacatagaacattgagttagcaatttacatgggctgtatgttatataagagaatgacatactgtggctaattcaacagtagatttattcttttagcctgcacaacagttgatcttttggctatgacaatttgtatggagggtacgatctaagttaagtgtgtcaaaagcaaggcttaggatttgttatgggagtagaatatatattgaattttgtatgaagaactatttgtttaaattatatagctgggatattttgccactgttaaaatggattcagaagaggtcctagaaaagtaagattagtgacatgtgtgggtttatatttagatatttaaggtgcattttcatagtgtggtaagaccttaagtaaaaggcacaatgggtactacagaattaaaatgtaggtctaacataatgccagttccactttaactttgtttttgcatttgaagaatgtatgtagcactttcctatatatttgtcacacattgaaaactggactgggtataactatgttataggaaagtagaaattgtattctttattttccatctttgttttctgttctacaaagttgatgcttaagcatcaagctgattttattggtcatgagaacaaatggatgtgatcatgaaggaatcagattccctatgtaaagcagtttaaaatggaattcaatgttcagtgctcaggtatgtagtaagtactgtagtcctgtgggggcaaatgtgtagatatttttaaacattttgccataattgcacaattttttgcatttttacctgatgtcattgtttcttataataaaaccttttctgattgaaaa

SEQ ID NO: 17 - protein, 575 aa, Homo sapiens
MDPFHACSILKQLKTMYDEGQLTDIVVEVDHGKTFSCHRNVLAAISPYFRSMFTSGLTESTQKEVRIVGVEAESMDLVLNYAYTSRVILTEANVQALFTAASIFQIPSIQDQCAKYMISHLDPQNSIGVFIFADHYGHQELGDRSKEYIRKKFLCVTKEQEFLQLTKDQLISILDSDDLNVDREEHVYESIIRWFEHEQNEREVHLPEIFAKCIRFPLMEDTFIEKIPPQFAQAIAKSCVEKGPSNTNGCTQRLGMTASEMIICFDAAHKHSGKKQTVPCLDIVTGRVFKLCKPPNDLREVGILVSPDNDIYIAGGYRPSSSEVSIDHKAENDFWMYDHSTNRWLSKPSLLRARIGCKLVYCCGKMYAIGGRVYEGDGRNSLKSVECYDSRENCWTTVCAMPVAMEFHNAVEYKEKIYVLQGEFFLFYEPQKDYWGFLTPMTVPRIQGLAAVYKDSIYYIAGTCGNHQRMFTVEAYDIELNKWTRKKDFPCDQSINPYLKLVLFQNKLHLFVRATQVTVEEHVFRTSRKNSLYQYDDIADQWMKVYETPDRLWDLGRHFECAVAKLYPQCLQKVL

SEQ ID NO: 18 - DNA, 3588 bp, Homo sapiens
ctggagactggaaggtccaagatcaagatactacagatttgatttctggacgttgaacatggtgtaggagtagaaaagcaacagggacggaaggagagaacttaccccttcaagcccttttataaggcactaaatcccatcattgagggcagagtcctcatagcctaatcacctcctaaatgctccatttcttaatattgttgcactgaggattaagcttcaacatgaattctgaagaggacacaaacatccaaaccatagcagtcaatgccttagcccttgatgttgctatcaacctgagattcggggatcaaggaaggacaggtaatagttaacctcttctgtgagaagtcagaaggtgatctctttaatgctttctttttaagaatttttcaaattgagactaattgcagaggttccagttgaccagcattcataggaatgaagacaaacacagagatggtgtgtctaagaaacttcaaaaggtgtagacctcctgactgaagcatattggatttatttaatttttttcactgtatttctgtcctcctacaagggaaagtcatgattacactaactgagctaaaatgcttagcagatgcccagtcatcttatcacatcttaaaaccatggtgggacgtcttctggtattacatcacactgatcatgctgctggtggccgtgctggccggagctctccagctgacgcagagcagggttctgtgctgtcttccatgcaaagtggaatttgacaatcactgtgccgtgccttgggacatcctgaaagccagcatgaacacatcctctaatcctgggacaccgcttccgctccccctccgaattcagaatgacctccaccgacagcagtactcctatattgatgccgtctgttacgagaaacagctccattggtttgcaaagtttttcccctatctggtgctcttgcacacgctcatctttgcagcctgcagcaacttttggcttcactaccccagtaccagttccaggctcgagcattttgtggccatccttcacaagtgcttcgattctccatggaccacccgcgccctttcagaaacagtggctgagcagtcagtgaggcctctgaaactctccaagtccaagattttgctttcgtcctcagggtgttcagctgacatagattccggcaaacagtcattgccctacccacagccaggtttggagtcagctggcatagaaagcccaacttccagtgtcctggacaagaaggagggtgaacaggccaaagccatctttgaaaaagtgaaaagattccgcatgcatgtggagcagaaggacatcatttatagagtatatctgaaacagataatagtcaaagtcattttgtttgtgctcatcataacttatgttccgtattttttaacccacatcactcttgaaatcgactgttcagttgatgtgcaggcttttacaggatataagcgctaccagtgtgtctattccttggcagaaatctttaaggtcctggcttcattttatgtcattttggttatactttatggtctgacctcttcctacagcctgtggtggatgctgaggagttccctgaagcaatattcctttgaggcgttaagagaaaaaagcaactacagtgacatccctgatgtcaagaatgactttgccttcatccttcatctggctgatcagtatgatcctctttattccaaacgcttctccatattcctatcagaggtcagtgagaacaaactgaaacagatcaacctcaataatgaatggacagttgagaaactgaaaagtaagcttgtgaaaaatgcccaggacaagatagaactgcatctttttatgctcaacggtcttccagacaatgtctttgagttaactgaaatggaagtgctaagcctggagcttatcccagaggtgaagctgccctctgcagtctcacagctggtcaacctcaaggagcttcgtgtgtaccattcatctctggtcgtagaccatcctgcactggcctttctagaggagaatttaaaaatcctccgcctgaaatttactgaaatgggaaaaatcccacgctgggtatttcacctcaagaatctcaaggaactttatctttcgggctgtgttctccctgaacagttgagtactatgcagttggagggctttcaggacttaaaaaatctaaggaccctgtacttgaagagcagcctctcccggatcccacaagttgttacagacctcctgccttcattgcagaaactgtcccttgataatgagggaagcaaactggttgtgttgaacaacttgaaaaagatggtcaatctgaaaagcctagaactgatcagctgtgacctggaacgcatcccacattccattttcagcctgaataatttgcatgagttagacctaagggaaaataaccttaaaactgtggaagagatcattagctttcagcatcttcagaatctttcctgcttaaagttgtggcacaataacattgcttatattcctgcacagattggggcattatctaacctagagcagctctctttggaccataataatattgagaatctgcccttgcagcttttcctatgcactaaactacattatttggatctaagctataaccacttgaccttcattccagaagaaatccagtatctgagtaatttgcagtactttgctgtgaccaacaacaatattgagatgctaccagatgggctgtttcagtgcaaaaagctgcagtgtttacttttggggaaaaatagcttgatgaatttgtcccctcatgtgggtgagctgtcaaaccttactcatctggagctcattggtaattacctggaaacacttcctcctgaactagaaggatgtcagtccctaaaacggaactgtctgattgttgaggagaacttgctcaatactcttcctctccctgtaacagaacgtttacagacgtgcttagacaaatgttgacttaaagaaaagagacccgtgtttcaaaatcatttttaaaagtatgctcggccgggcgtggtggctcatgcctataatcccagcactttgggaggccaagatgggcggattgcttgaggtcaggagttcgagaccagtctggccaacctggtgaaaccccatctctgctaaaactacaaaaaaattagccaggcgtggtggcgtgcgcctgtaatcccagctacttgggaggctgacgcaggggaattgcttgaaccagggaggtggaggttgcagtgagccgagattgtgccactgtacaccagcctgggtgacagagcaagactcttatctcaaaaaaaaaaaaaaatgctccagggctttaaatgagaagtaaaattttctaagttaataaagatgaagaatgggtgactattatgatgaaccataactaaatgtcttattaaagcaactgagtgtctagccctaaattaaccaggtaaaaactgttaacactaacctgaagttttgtgaataactgttctttaacttattgagatgttgcaagaaatgcacatccagggtggactgggagctatgaaatgactaaattcctccttgcagtgtttacct

SEQ ID NO: 19 - protein, 803 aa, Homo sapiens
MITLTELKCLADAQSSYHILKPWWDVFWYYITLIMLLVAVLAGALQLTQSRVLCCLPCKVEFDNHCAVPWDILKASMNTSSNPGTPLPLPLRIQNDLHRQQYSYIDAVCYEKQLHWFAKFFPYLVLLHTLIFAACSNFWLHYPSTSSRLEHFVAILHKCFDSPWTTRALSETVAEQSVRPLKLSKSKILLSSSGCSADIDSGKQSLPYPQPGLESAGIESPTSSVLDKKEGEQAKAIFEKVKRFRMHVEQKDIIYRVYLKQIIVKVILFVLIITYVPYFLTHITLEIDCSVDVQAFTGYKRYQCVYSLAEIFKVLASFYVILVILYGLTSSYSLWWMLRSSLKQYSFEALREKSNYSDIPDVKNDFAFILHLADQYDPLYSKRFSIFLSEVSENKLKQINLNNEWTVEKLKSKLVKNAQDKIELHLFMLNGLPDNVFELTEMEVLSLELIPEVKLPSAVSQLVNLKELRVYHSSLVVDHPALAFLEENLKILRLKFTEMGKIPRWVFHLKNLKELYLSGCVLPEQLSTMQLEGFQDLKNLRTLYLKSSLSRIPQVVTDLLPSLQKLSLDNEGSKLVVLNNLKKMVNLKSLELISCDLERIPHSIFSLNNLHELDLRENNLKTVEEIISFQHLQNLSCLKLWHNNIAYIPAQIGALSNLEQLSLDHNNIENLPLQLFLCTKLHYLDLSYNHLTFIPEEIQYLSNLQYFAVTNNNIEMLPDGLFQCKKLQCLLLGKNSLMNLSPHVGELSNLTHLELIGNYLETLPPELEGCQSLKRNCLIVEENLLNTLPLPVTERLQTCLDKC